(19) United States
(12) Patent Application Publication
(10) Pub. No.: US 2005/0255560 A1
(43) Pub. Date: Nov. 17, 2005

(54) Ztnf11, a tumor necrosis factor

(76) Inventors: Zeren Gao, Redmond, WA (US); Paul O. Sheppard, Granite Falls, WA (US); Brian A. Fox, Seattle, WA (US); James L. Holloway, Seattle, WA (US); Stephen R. Jaspers, Edmonds, WA (US)

Correspondence Address: Robyn Adams;ZymoGenetics, Inc., 1201 Eastlake Avenue East, Seattle, WA, 98102, US

(21) Appl. No.: 10/994,820

(22) Filed: Nov. 22, 2004

Related U.S. Application Data
(60) Provisional application No. 60/524,163, filed on Nov. 21, 2003.

(51) Int. Cl. C07H 21/04; C12P 21/02; C07K 14/525; C07K 16/24

(52) U.S. Cl. 435/69.5; 435/320.1; 435/325; 530/351; 530/388.23; 536/23.5

(57) ABSTRACT

Novel tumor necrosis factor ligand polypeptides, polynucleotides encoding the polypeptides, and related compositions and methods are disclosed. The polypeptides may be used within methods relating to immune response, and may also be used in the development of immuno-regulatory therapeutics. Also provided are antibodies, binding proteins, agonists and antagonists of the ligand polypeptides.

### CROSS REFERENCE TO RELATED APPLICATIONS

This application claims the benefit of U.S. Provisional Application Ser. No. 60/524,163, filed Nov. 21, 2003, which is herein incorporated by reference.

### BACKGROUND OF THE INVENTION

Cellular interactions which occur during an immune response are regulated by members of several families of cell surface receptors and their respective ligands, including the tumor necrosis factor (TNF) family. Several members of this family regulate interactions between different hematopoietic cell lineages (Smith et al., The TNF Receptor Superfamily of Cellular and Viral Proteins: Activation, Costimulation and Death, 76:959-62, 1994; Cosman, Stem Cells 12:440-55, 1994). In general, the members of the TNF family mediate interactions between different hematopoietic cells, such as T cell/B cell, T cell/monocyte and T cell/T cell interactions. The result of this two-way communication can be stimulatory or inhibitory, depending on the target cell or the activation state. TNF ligands are involved in regulation of cell proliferation, activation and differentiation, including control of cell survival or death by apoptosis or cytotoxicity. Differences in TNF receptor (TNFR) distribution, kinetics of induction and requirements for induction, support the concept of a defined role for each of the TNF ligands in T cell-mediated immune responses.

The TNF ligand family is composed of a number of type II integral membrane glycoproteins. Members of this family, with the exception of nerve growth factor (NGF) and LT-α, contain an N-terminal cytoplasmic region, a single transmembrane region, a linker region and a 150 to 170 amino acid residue C-terminal receptor-binding domain. The tertiary structure of the C-terminal receptor-binding domain has been determined to be a β-sandwich. Members of this family, with the exception of NGF, share approximately 20% sequence homology within this extracellular receptor-binding domain, and little to no homology within the linker, transmembrane and cytoplasmic regions. The ligands within this family are biologically active as trimeric or multimeric complexes. This group includes TNF, LT-α, LT-β, CD27L, CD30L, CD40L, 4-1BBL, OX40L, FasL (Cosman, ibid.; Lotz et al., J. Leukoc. Biol. 60:1-7, 1996), TRAIL or apo-2 ligand (Wiley et al., Immunity 3:673-82, 1995), and TNF γ(WO 96/14328). The presence of a transmembrane region indicates that the ligands are membrane-associated. Soluble ligand forms have been identified for TNFα, LT-α and FasL. It is not known whether a specific protease cleaves each ligand, releasing it from the membrane, or whether one protease serves the same function for all TNF ligand family members. TACE (TNF-alpha converting enzyme) has been shown to cleave TNFα (Moss et al., Nature 385:733-36, 1997; Black et al., Nature 385:729-33, 1997).

The TNFR family is made up of type I integral membrane glycoproteins, including p75 NGFR, p55 TNFR-I, p75 TNFR-II, TNFR-RP/TNFR-Eff, CD27, CD30, CD40, 4-1BB, OX40, FAS/APO-1 (Cosman, ibid.; Lotz et al., ibid.), HVEM (Montgomery et al., Cell 87:427-36, 1996), WSL-1 (Kitson et al., Nature 384:372-75, 1996) also known as DR3 (Chinnaiyan et al., Science 274:990-92, 1996), DR4 (Pan et al., Science 276:111-13, 1997), a TNF receptor protein described in WO 96/28546 now known as osteoprotegerin (OPG, Simonet et al., Cell 89:309-19, 1997), CAR1, found in chicken (Brojatsch et al., Cell 87:845-55, 1996) plus several viral open reading frames encoding TNFR-related molecules. NGFR, TNFR-I, CD30, CD40, 4-1BB, DR3, DR4 and OX40 are mainly restricted to cells of the lymphoid/hematopoietic system.

The interaction of one member of the TNF ligand family, TNF, and its receptor, has been shown to be essential to a broad spectrum of biological processes and pathologies. In particular, the receptor-ligand pair has a variety of immunomodulatory properties, including mediating immune regulation, immunostimulation and moderating graft rejection. An involvement has also been demonstrated in inflammation, necrosis of tumors (Gray et al., Nature 312:721-24, 1984), septic shock (Tracy et al., Science 234:470-74, 1986) and cytotoxicity. TNF promotes and regulates cellular proliferation and differentiation (Tartalgia et al., J. Immunol. 151:4637-41, 1993. In addition, TNF and its receptor are also involved in apoptosis.

The X-ray crystallographic structures have been resolved for human TNF (Jones et al., Nature 388:225-28, 1989), LT-β (Eck et al., J. Biol. Chem. 267:2119-22, 1992), and the LT-β/TNFR complex (Banner et al., Cell 73:431-35, 1993). This complex features three receptor molecules bound symmetrically to one LT-β trimer. A model of trimeric ligand binding through receptor oligomerization has been proposed to initiate signal transduction pathways. The identification of biological activity of several TNF members has been facilitated through use of monoclonal antibodies specific for the corresponding receptor. These monoclonal antibodies tend to be stimulatory when immobilized and antagonistic in soluble form. This is further evidence that receptor crosslinking is a prerequisite for signal transduction in both the receptor and ligand families. Importantly, the use of receptor-specific monoclonal antibodies or soluble receptors in the form of multimeric Ig fusion proteins has been useful in determining biological function in vitro and in vivo for several family members. Soluble receptor-Ig fusion proteins have been used successfully in the cloning of the cell surface ligands corresponding to the CD40, CD30, CD27, 4-1BB and Fas receptors.

The members of the TNF ligand family exist mainly as type II membrane glycoproteins, biologically active as trimeric or multimeric complexes. Although most ligands are synthesized as membrane-bound proteins, soluble forms can be generated by limited proteolysis. For some receptors, solublization is necessary for activity, while for others, their activity is inhibited upon cleavage.

A Proliferation Inducing Ligand (APRIL) is an example of a tumor necrosis factor ligand known to be active in its soluble form (reviewed in Medema et al. Cell Death and Diff. 10: 1121-25). APRIL is unique in that it is cleaved intracellularly and produced by the cell secretion pathway, not through cleavage of a membrane bound form. APRIL was isolated based on its ability to stimulate the proliferation of tumor cells in vitro. Experiments utilizing transgenic mice expressing APRIL suggest a role for this ligand in stimulating T-cells. This ligand is known to bind to two members of the TNFR family: BCMA and TACI. However, there is experimental evidence for at least one further receptor for APRIL. Specifically, the Jurkat human leukemia T-cell line is susceptible to APRIL stimulation but neither BCMA nor TACI is detectable in Jurkat cells by Northern blot analysis (Medema et al., ibid).

Inflammation normally is a localized, protective response to trauma or microbial invasion that destroys, dilutes, or walls-off the injurious agent and the injured tissue. Diseases characterized by inflammation are significant causes of morbidity and mortality in humans. While inflammation commonly occurs as a defensive response to invasion of the host by foreign material, it is also triggered by a response to mechanical trauma, toxins, and neoplasia. Excessive inflammation caused by abnormal recognition of host tissue as foreign, or prolongation of the inflammatory process, may lead to inflammatory diseases such as diabetes, asthma, atherosclerosis, cataracts, reperfusion injury, cancer, post-infectious syndromes such as in infectious meningitis, and rheumatic fever and rheumatic diseases such as systemic lupus erythematosus and rheumatoid arthritis. Thus, there is a need to produce agents that inhibit inflammation in many such diseases.

The demonstrated in vivo activities of these TNF ligand family members illustrate the enormous clinical potential of, and need for, other TNF ligands, ligand agonists and antagonists, and TNF receptors. The present invention addresses this need by providing a novel TNF ligand and related compositions and methods.

### SUMMARY OF THE INVENTION

Within one aspect, the invention provides an isolated polypeptide comprising the amino acid sequence of residues 116 to 309 of SEQ ID NO:2. Within an embodiment, the polypeptide comprises the amino acid sequence selected from: a) residues 86 to 309 of SEQ ID NO:2; b) residues 79 to 309 of SEQ ID NO:2; c) residues 51 to 309 of SEQ ID NO:2; d) residues 115 to 309 of SEQ ID NO:2; and e) residues 1 to 309 of SEQ ID NO:2, wherein the polypeptide is at least 80% identical to the amino acid sequence of a), b), c), d),or e). Within another embodiment, the polypeptide is at least 85% identical to the amino acid sequence of a), b), c), or d). Within another embodiment, the polypeptide forms a multimer. Within another embodiment, the multimer is selected from: a) a homodimer; b) a heterodimer; c) a homotrimer; d) a heterodimer; e) a homomultimer; and f) a heteromultimer. Within another embodiment the polypeptide binds a TNF receptor. Within another embodiment the polypeptide is covalently linked to an affinity tag. Within another embodiment, the polypeptide is covalently linked to an immunoglobulin constant region. Within another embodiment, the polypeptide induces an inflammatory response.

Within another aspect, the invention provides an isolated polynucleotide, wherein the polynucleotide encodes a polypeptide comprising the amino acid sequence selected from: a) residues 116 to 309 of SEQ ID NO:2; b) residues 115 to 309 of SEQ ID NO:2; c) residues 86 to 309 of SEQ ID NO:2; d) residues 79 to 309 of SEQ ID NO:22; e) residues 51 to 309 of SEQ ID NO:2; and f) residues 1 to 309 of SEQ ID NO:2. Within an embodiment, the polypeptide consists of the amino acid sequence. Within another embodiment the invention provides an isolated polynucleotide, wherein the polynucleotide encodes the polypeptide comprising the amino acid sequence selected from: a) residues 116 to 309 of SEQ ID NO:2; b) residues 115 to 309 of SEQ ID NO:2; c) residues 86 to 309 of SEQ ID NO:2; d) residues 79 to 309 of SEQ ID NO:22; e) residues 51 to 309 of SEQ ID NO:2; and f) residues 1 to 309 of SEQ ID NO:2.

Within another aspect, the invention provides an expression vector comprising the following operably linked elements: a transcription promoter; a DNA segment encoding a polypeptide comprising residues 116 to 309 of SEQ ID NO:2; and a transcription terminator. Within an embodiment, the polypeptide comprises an affinity tag or an immunoglobulin constant region.

Within another aspect, the invention provides an antibody that specifically binds to a polypeptide comprising residues 116 to 309 of SEQ ID NO:2. Within an embodiment, the antibody is a polyclonal antibody. Within an embodiment, the antibody is a monoclonal antibody. Within another aspect, the specifically binds to the polypeptide comprising the amino acid sequence selected from: a) residues 116 to 309 of SEQ ID NO:2; b) residues 115 to 309 of SEQ ID NO:2; c) residues 86 to 309 of SEQ ID NO:2; d) residues 79 to 309 of SEQ ID NO:22; e) residues 51 to 309 of SEQ ID NO:2; and f) residues 1 to 309 of SEQ ID NO:2.

Within another aspect, the invention provides a method of producing an antibody or an antibody fragment, comprising the following steps in order: inoculating an animal with a polypeptide selected from the group consisting of: a) a polypeptide consisting of the amino acid sequence from residue 116 to 309 of SEQ ID NO:2; b) a polypeptide consisting of the amino acid sequence from residue 86 to 309 of SEQ ID NO:2; c) a polypeptide consisting of the amino acid sequence from reside 79 to 309 of SEQ ID NO:2; d) a polypeptide consisting of the amino acid sequence from residue 51 to 309 of SEQ ID NO:2; and e) a polypeptide consisting of the amino acid sequence from residue 1 to 309 of SEQ ID NO:2; wherein the polypeptide elicits an immune response in the animal to produce the antibody; and isolating the antibody from the animal. Within an embodiment, the antibody binds to residues 1 to 309 of SEQ ID NO:2.

Within another aspect the invention provides a method of inhibiting or reducing inflammation associated with an autoimmune disease, comprising administering to a mammal with the autoimmune disease a therapeutic amount of an antibody, wherein the antibody specifically binds to the polypeptide selected from: a) residues 116 to 309 of SEQ ID NO:2; b) residues 115 to 309 of SEQ ID NO:2; c) residues 86 to 309 of SEQ ID NO:2; d) residues 79 to 309 of SEQ ID NO:22; e) residues 51 to 309 of SEQ ID NO:2; and f) residues 1 to 309 of SEQ ID NO:2. Within an embodiment, the autoimmune disease is systemic lupus erythomatosis, myasthenia gravis, multiple sclerosis, or rheumatoid arthritis.

Within another aspect the invention provides a method of inhibiting or reducing inflammation associated with an asthma, bronchitis, or emphysema, comprising administering to a mammal with the asthma, bronchitis, or emphysema a therapeutic amount of an antibody, wherein the antibody specifically binds to the polypeptide selected from: a) residues 116 to 309 of SEQ ID NO:2; b) residues 115 to 309 of SEQ ID NO:2; c) residues 86 to 309 of SEQ ID NO:2; d) residues 79 to 309 of SEQ ID NO:22; e) residues 51 to 309 of SEQ ID NO:2; and f) residues 1 to 309 of SEQ ID NO:2.

Within another aspect the invention provides a method of reducing joint pain, swelling, stiffness, or anemia, comprising comprising administering to a mammal with the joint pain, swelling, stiffness, or anemia a therapeutic amount of an antibody, wherein the antibody specifically binds to the polypeptide selected from: a) residues 116 to 309 of SEQ ID NO:2; b) residues 115 to 309 of SEQ ID NO:2; c) residues 86 to 309 of SEQ ID NO:2; d) residues 79 to 309 of SEQ ID NO:22; e) residues 51 to 309 of SEQ ID NO:2; and f) residues 1 to 309 of SEQ ID NO:2.

Within another aspect the invention provides an antibody or antibody fragment that specifically binds to the polypeptide comprising amino acid residues 116 to 309 of SEQ ID NO:2, wherein the antibody or antibody fragment is: a) a polyclonal antibody, b) a monoclonal antibody; c) a murine monoclonal antibody; and d) a humanized antibody derived from c).

Within another aspect, the invention provides an isolated polypeptide comprising the amino acid sequence of residues 116 to 267 of SEQ ID NO:2. Within an embodiment, the polypeptide comprises the amino acid sequence selected from: a) residues 116 to 267 of SEQ ID NO:2; and b) residues 116 to 272 of SEQ ID NO:2, wherein the polypeptide is at least 80 % identical to the amino acid sequence of a) or b). Within another embodiment, the polypeptide is at least 85 % identical to the amino acid sequence of a) or b). Within another embodiment, the polypeptide forms a multimer. Within another embodiment, the multimer is selected from: a) a homodimer; b) heterodimer; c) a homotrimer; d) a heterodimer; e) a homomultimer; and f) a heteromultimer. Within another embodiment the polypeptide binds a TNF receptor. Within another embodiment the polypeptide is covalently linked to an affinity tag. Within another embodiment, the polypeptide is covalently linked to an immunoglobulin constant region. Within another embodiment, the polypeptide induces an inflammatory response.

Within another aspect, the invention provides an isolated polynucleotide, wherein the polynucleotide encodes a polypeptide comprising the amino acid sequence selected from: a) residues 116 to 267 of SEQ ID NO:2; and b) residues 116 to 272 of SEQ ID NO:2. Within an embodiment, the polypeptide consists of the amino acid sequence. Within another embodiment the invention provides an isolated polynucleotide, wherein the polynucleotide encodes the polypeptide comprising the amino acid sequence selected from: a) residues 116 to 267 of SEQ ID NO:2; and b) residues 116 to 272 of SEQ ID NO:2.

Within another aspect, the invention provides an expression vector comprising the following operably linked elements: a transcription promoter; a DNA segment encoding a polypeptide comprising residues 116 to 267 of SEQ ID NO:2; and a transcription terminator. Within an embodiment, the polypeptide comprises an affinity tag or an immunoglogulin constant region.

Within another aspect, the invention provides an antibody that specifically binds to a polypeptide comprising residues 116 to 267 of SEQ ID NO:2. Within an embodiment, the antibody is a polyclonal antibody. Within an embodiment, the antibody is a monoclonal antibody. Within another aspect, the specifically binds to the polypeptide comprising the amino acid sequence selected from: a) residues 116 to 267 of SEQ ID NO:2; and b) residues 116 to 272 of SEQ ID NO:2.

Within another aspect, the invention provides a method of producing an antibody or an antibody fragment, comprising the following steps in order: inoculating an animal with a polypeptide selected from the group consisting of: a) a polypeptide consisting of the amino acid sequence from residue 116 to 267 of SEQ ID NO:2; and b) a polypeptide consisting of the amino acid sequence from residue 116 to 272 of SEQ ID NO:2; wherein the polypeptide elicits an immune response in the animal to produce the antibody; and isolating the antibody from the animal. Within an embodiment, the antibody binds to residues 116 to 267 of SEQ ID NO:2.

Within another aspect the invention provides a method of inhibiting or reducing inflammation associated with an autoimmune disease, comprising administering to a mammal with the autoimmune disease a therapeutic amount of an antibody, wherein the antibody specifically binds to the polypeptide selected from: a) residues 116 to 267 of SEQ ID NO:2; and b) residues 116 to 272 of SEQ ID NO:2. Within an embodiment, the autoimmune disease is systemic lupus erythomatosis, myasthenia gravis, multiple sclerosis, or rheumatoid arthritis.

Within another aspect the invention provides a method of inhibiting or reducing inflammation associated with an asthma, bronchitis, or emphysema, comprising administering to a mammal with the asthma, bronchitis, or emphysema a therapeutic amount of an antibody, wherein the antibody specifically binds to the polypeptide selected from: a) residues 116 to 267 of SEQ ID NO:2; and b) residues 116 to 272 of SEQ ID NO:2.

Within another aspect the invention provides a method of reducing joint pain, swelling, stiffness, or anemia, comprising administering to a mammal with the joint pain, swelling, stiffness, or anemia a therapeutic amount of an antibody, wherein the antibody specifically binds to the polypeptide selected from: a) residues 116 to 267 of SEQ ID NO:2; and b) residues 116 to 272 of SEQ ID NO:2.

Within another aspect the invention provides an antibody or antibody fragment that specifically binds to the polypeptide comprising amino acid residues 116 to 267 of SEQ ID NO:2, wherein the antibody or antibody fragment is: a) a polyclonal antibody, b) a monoclonal antibody; c) a murine monoclonal antibody; and d) a humanized antibody derived from c).

Within one aspect, the invention provides an isolated polypeptide comprising the amino acid sequence of residues 51 to 120 of SEQ ID NO:12. Within an embodiment, the polypeptide comprises the amino acid sequence selected from: a) residues 51 to 120 of SEQ ID NO:12; and b) residues 1 to 120 of SEQ ID NO:12;, wherein the polypeptide is at least 80% identical to the amino acid sequence of a) or b). Within another embodiment, the polypeptide is at least 85% identical to the amino acid sequence of a) or b). Within another embodiment, the polypeptide forms a multimer. Within another embodiment, the multimer is selected from: a) a homodimer; b) heterodimer; c) a homotrimer; d) a heterodimer; e) a homomultimer; and f) a heteromultimer. Within another embodiment the polypeptide binds a TNF receptor. Within another embodiment the polypeptide is covalently linked to an affinity tag. Within another embodiment, the polypeptide is covalently linked to an immunoglobulin constant region. Within another embodiment, the polypeptide induces an inflammatory response.

Within another aspect, the invention provides an isolated polynucleotide, wherein the polynucleotide encodes a polypeptide comprising the amino acid sequence selected from: a) residues 51 to 120 of SEQ ID NO:12; and b) residues 51 to 120 of SEQ ID NO:12. Within an embodiment, the polypeptide consists of the amino acid sequence. Within another embodiment the invention provides an isolated polynucleotide, wherein the polynucleotide encodes the polypeptide comprising the amino acid sequence selected from: a) residues 51 to 120 of SEQ ID NO:12; and b) residues 1 to 120 of SEQ ID NO:12;.

Within another aspect, the invention provides an expression vector comprising the following operably linked elements: a transcription promoter; a DNA segment encoding a polypeptide comprising residues 51 to 120 of SEQ ID NO:12; and a transcription terminator. Within an embodiment, the polypeptide comprises an affinity tag or an immunoglogulin constant region.

Within another aspect, the invention provides an antibody that specifically binds to a polypeptide comprising residues 51 to 120 of SEQ ID NO: 12. Within an embodiment, the antibody is a polyclonal antibody. Within an embodiment, the antibody is a monoclonal antibody. Within another aspect, the specifically binds to the polypeptide comprising the amino acid sequence selected from: a) residues 51 to 120 of SEQ ID NO:12; and b) residues 1 to 120 of SEQ ID NO:12.

Within another aspect, the invention provides a method of producing an antibody or an antibody fragment, comprising the following steps in order: inoculating an animal with a polypeptide selected from the group consisting of: a) a polypeptide consisting of the amino acid sequence from residue 51 to 120 of SEQ ID NO:12; and b) a polypeptide consisting of the amino acid sequence from residue 1 to 120 of SEQ ID NO:12; wherein the polypeptide elicits an immune response in the animal to produce the antibody; and isolating the antibody from the animal. Within an embodiment, the antibody binds to residues 1 to 120 of SEQ ID NO:12.

Within another aspect the invention provides a method of limiting the reduction of inflammation associated with an autoimmune disease, comprising administering to a mammal with the autoimmune disease a therapeutic amount of an antibody, wherein the antibody specifically binds to the polypeptide selected from: a) residues 51 to 120 of SEQ ID NO:12; and b) residues 1 to 120 of SEQ ID NO:12. Within an embodiment, the autoimmune disease is systemic lupus erythomatosis, myasthenia gravis, multiple sclerosis, or rheumatoid arthritis.

Within another aspect the invention provides a method limiting the reduction of inflammation associated with an asthma, bronchitis, or emphysema, comprising administering to a mammal with the asthma, bronchitis, or emphysema a therapeutic amount of an antibody, wherein the antibody specifically binds to the polypeptide selected from: a) residues 51 to 120 of SEQ ID NO:12; and b) residues 1 to 120 of SEQ ID NO:12.

Within another aspect the invention provides a method of limiting the reduction of joint pain, swelling, stiffness, or anemia, comprising comprising administering to a mammal with the joint pain, swelling, stiffness, or anemia a therapeutic amount of an antibody, wherein the antibody specifically binds to the polypeptide selected from: a) residues 51 to 120 of SEQ ID NO:12; and b) residues 1 to 120 of SEQ ID NO:12.

Within another aspect the invention provides an antibody or antibody fragment that specifically binds to the polypeptide comprising amino acid residues 51 to 120 of SEQ ID NO: 12, wherein the antibody or antibody fragment is: a) a polyclonal antibody, b) a monoclonal antibody; c) a murine monoclonal antibody; and d) a humanized antibody derived from c).

Within another aspect, the polypeptides taught herein are at least 85%, 90%, 95%, 96%, 97%, 98%, or 99% identical to the polypeptides as shown in SEQ ID NOs: 2, 12, and 15.

Within another aspect the invention provides a pharmaceutical composition comprising a polypeptide according to comprising amino acid residues 116 to 309 of SEQ ID NO: 2; amino acid residues 116 to 267 of SEQ ID NO:2, or residues 51 to 120 of SEQ ID NO: 12.

Within another aspect the invention provides for methods of detecting the polynucleotides and polypeptides of the present invention to detect and diagnose diseases, including but not limited to autoimmune disease, such as systemic lupus erythomatosis, myasthenia gravis, multiple sclerosis, and rheumatoid arthritis, and asthma, bronchitis, or emphysema.

Within another aspect the invention provides a soluble polypeptide from residue 136 to residue 309 of SEQ ID NO:2.

### DESCRIPTION OF THE INVENTION

Prior to setting forth the invention, it may be helpful to an understanding thereof to set forth definitions of certain terms to be used hereinafter:

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985), substance P, Flag™ peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Pharmacia Biotech, Piscataway, N.J.).

The term “allelic variant” is used herein to denote any of two or more alternative forms of a gene occupying the same chromosomal locus. Allelic variation arises naturally through mutation, and may result in phenotypic polymorphism within populations. Gene mutations can be silent (i.e., no change in the encoded polypeptide), or may encode polypeptides having altered amino acid sequence. The term “allelic variant” is also used herein to denote a protein encoded by an allelic variant of a gene. Also included are the same protein from the same species which differs from a reference amino acid sequence due to allelic variation. Allelic variation refers to naturally occurring differences among individuals in genes encoding a given protein.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complement/anti-complement pair denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁻⁹ M.

The term “complements” of polynucleotide molecules denotes polynucleotide molecules having a complementary base sequence and reverse orientation as compared to a reference sequence. For example, the sequence 5′ ATGCACGGG 3′ is complementary to 5′ CCCGTGCAT 3′.

The term “contig” denotes a polynucleotide that has a contiguous stretch of identical or complementary sequence to another polynucleotide. Contiguous sequences are said to “overlap” a given stretch of polynucleotide sequence either in their entirety or along a partial stretch of the polynucleotide. For example, representative contigs to the polynucleotide sequence 5′-ATGGCTTAGCTT-3′ are 5′-TAGCTTgagtct-3′ and 3′-gtcgacTACCGA-5′.

The term “degenerate as applied to a nucleotide sequence such as a probe or primer, denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” denotes a DNA molecule, linear or circular, that comprises a segment encoding a polypeptide of interest operably linked to additional segments that provide for its transcription. Such additional segments may include promoter and terminator sequences, and optionally one or more origins of replication, one or more selectable markers, an enhancer, a polyadenylation signal, and the like. Expression vectors are generally derived from plasmid or viral DNA, or may contain elements of both.

The term “isolated” when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked” as applied to nucleotide segments indicates that the segments are arranged so that they function in concert for their intended purposes, e.g., transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

The term “polynucleotide” denotes a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

The term “polypeptide” as used herein is a polymer of amino acid residues joined by peptide bonds, whether produced naturally or synthetically.

Polypeptides of less than about 10 amino acid residues are commonly referred to as “peptides”.

The term “promoter” denotes a portion of a gene containing DNA sequences that provide for the binding of RNA polymerase and initiation of transcription. Promoter sequences are commonly, but not always, found in the 5′ non-coding regions of genes.

The term “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” as used herein denotes a cell-associated protein, or a polypeptide subunit of such protein, that binds to a bioactive molecule (the “ligand”) and mediates the effect of the ligand on the cell. Binding of ligand to receptor results in a change in the receptor (and, in some cases, receptor multimerization, i.e., association of identical or different receptor subunits) that causes interactions between the effector domain(s) of the receptor and other molecule(s) in the cell. These interactions in turn lead to alterations in the metabolism of the cell. Metabolic events that are linked to receptor-ligand interactions include gene transcription, phosphorylation, dephosphorylation, cell proliferation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” as used herein denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “soluble receptor” or “ligand” as used herein denotes a receptor or a ligand polypeptide that is not bound to a cell membrane. Soluble receptors are most commonly ligand-binding receptor polypeptides that lack transmembrane and cytoplasmic domains. Soluble ligands are most commonly receptor-binding polypeptides that lack transmembrane and cytoplasmic domains. Soluble receptors or ligands can comprise additional amino acid residues, such as affinity tags that provide for purification of the polypeptide or provide sites for attachment of the polypeptide to a substrate. Many cell-surface receptors and ligands have naturally occurring, soluble counterparts that are produced by proteolysis or translated from alternatively spliced mRNAs. Receptor and ligand polypeptides are said to be substantially free of transmembrane and intracellular polypeptide segments when they lack sufficient portions of these segments to provide membrane anchoring or signal transduction, respectively.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the identification of a DNA sequence (SEQ ID NO: 1) and corresponding polypeptide sequence (SEQ ID NO:2) as a novel member of the Tumor Necrosis Factor ligand family, Ztnf11. This new TNF ligand, has homology to members of the tumor necrosis factor ligand family, and in particular to LT-Beta, Ztnf4, and CD27 ligand. See Shu H.-B., et all., J. Leukoc. Biol. 65:680-683(1999); Browning J. L., et al., Cell 72:847-856(1993); and Goodwin R. G., et al., Cell 73:447-456(1993).

This novel tumor necrosis factor may be involved in modulating an immune response, hematopoeisis, inflammation, cellular deficiencies, abnormal cellular proliferation, apoptosis, cancers, or in treating inflammatory conditions. The ligand has been designated Ztnf11. Ztnf11 is a member of the typeII membrane protein family, which includes TNFa. Ztnf11 mRNA is expressed by a number of monocyte and activated monocyte cell types including stimulated U937 cells and HL-60 cells. In addition, the protein is produced by these cells, as evidenced by western blots using cell lysates and antibodies to MBP-fusion produced protein. Zntf11 expressed on the surface of activated monocytes (and potentially activated by an inflammatory environment) will likely contribute to the inflammatory process as do other typeII membrane proteins like TNFa. Under conditions of monocytopenia, and lymphopenia (for example, hematopoietic stem cell donors, stem cell transplant recipients, childhood severe acute respiratory syndrome (SARS), Aplastic anemias, Hairy cell Leukaemia, purulent otitis media, and virally challenged patients) soluble ztnf11 molecules will be useful in activating resting monocytes, and likely other lymphocytes, as well as to help boost the immune system.

Antibodies to a ztnf11 soluble domain recognized protein of the correct full-length molecular weight from cell lysates of U937 and HL-60 cells. See Example 19. Ztnf11 expression as a membrane bound protein on monocytes indicates that recombinant ztnf11 can be useful for the generation of antibodies to ztnf11, which will be able to target the native protein on monocytes. In addition, these antibodies are useful for recognizing and inhibiting or deleting activated monocytes. Under pathological monocyte in-vivo conditions, such as autoimmune diseases (psoriasis, systemic lupus, systemic sclerosis, systemic lupus erythomatosis, myasthenia gravis, multiple sclerosis, rheumatoid arthritis, bronchitis, emphysema, end stage renal failure, renal disease, glomerulo-nephritis, vasculitis, nephritis, pyrlonephritis, renal neoplasms, multiple myelomas, lymphomas, light chain neuropathy or amyloidosis) or other inflammatory disease such as asthma, COPD, and ARDS, inflammatory bowel disease such as ulcerative colitis or Crohn's disease. Antibodies to ztnf11 will be useful in regulating the inflammatory effects of activated moncytes. Especially relevant to disease will be those diseases which affect areas and cell types of the body which express ztnf11 such as stomach, small intestine and testis, including Inflammatory Bowel Disease, Irritable Bowel Syndrome, and Chronic Diarrhea.

In a peripheral blood fractions, ztnf11×1 mRNA is expressed in many samples, including activated and resting CD4+ T-helper cells and CD19+ B-cells, resting CD8+ cytotoxic T-cells, mononuclear cells and possibly activated mononuclear cells. Resting CD14+ monocyte cells and activated CD8+ cytotoxic T-cells are negative for ztnf11×1 in this assay. Ztnf11×2 mRNA is not expressed in any of these peripheral blood fractions.

Novel Ztnf11 ligand-encoding polynucleotides and polypeptides of the present invention were initially identified based on a combination of characteristics specific to the TNF ligand family of proteins. These characteristics include gene structure, identification of a transmembrane anchor, protein size, chromosomal location and sequence similarity to the TNF ligands. Using this information, a human cDNA (SEQ ID NO:1) was identified as a family member of TNF ligands. Analysis of the cDNA sequence (SEQ ID NO: 1) revealed an open reading frame encoding the 309 amino acids Ztnf11 amino acid sequence (SEQ ID NO: 2). The Ztnf11 polypeptide comprises an amino terminal transmembrane domain from residue 27 to residue 50 of SEQ ID NO:2. As a Type II protein, the intracellular domain of the protein is from residue 1 to 26 of SEQ ID NO:2, and the extracellular domain is from residue 51 to 309 of SEQ ID NO:2 (SEQ ID NO:17). An additional cDNA sequence was identified as SEQ ID NO: 11, which revealed an additional open reading frame encoding the 120 amino acids Ztnf11×2 amino acid sequence (SEQ ID NO: 12). The Ztnf11 polypeptide comprises an amino terminal transmembrane domain from residue 27 to residue 50 of SEQ ID 12. As a Type II protein, the intracellular domain of the protein is from residue 1 to 26 of SEQ ID NO:12, and the extracellular domain is from residue 51 to 120 of SEQ ID NO.12, or the polypeptide of SEQ ID NO:16. One of ordinary skill in the art will recognize that these domain boundaries are approximate, and can be ±2 or more amino acids different.

Analysis of the gene structure of Ztnf11 show that it has similarities with other TNF ligands. The first exon of the polynucleotide sequence spans nucleotides 1 to 169 of SEQ ID NO:1. The second exon of the polypeptide sequence spans nucleotides 170 to 230 of SEQ ID NO:1. The third exon of the polypeptide sequence spans nucleotides 231 to 982 of SEQ ID NO:1. Other members of the TNF ligand family which share the three exon structure include TNFP, OX4oL, CD27L, 41BBL, and GITRL. Furthermore, the intron phases of these TNF ligands are conserved, which implies an evolutionary relationship between the family members.

Those skilled in the art will recognize that these domain boundaries are approximate, and are based on alignments with known proteins and predictions of protein folding.

Most proteins which are members of the TNF family can be recognized by a conserved central hydrophobic TNF consensus motif represented by:
[GLIVMFY]-X-[WLIVMFY]-X-X-X-G-[LIVMFY]-[RFY]-[ALIVMFY]-[LIVMFY]

(SEQ ID NO:4). In Ztnf11, this motif is represented by GIWSELGLRAY (SEQ ID NO:5), amino acids 193 to 203 of SEQ ID NO:2.

Using the crystal structure of APO2L and DR5 (a TNF and TNF receptor in PDB: 1DU3), a peptide loop of APO2L is observed to interact with the TNF receptor. Given the homology between RANKL and APO2L, the 3D structure of RANKL interacting with RANK is likely to be very similar. As such, a homologous peptide loop of Ztnf11 may interact with a TNF receptor in an analogous fashion. The loop may form a disulfide bond, which would constrain the peptide and force it into a conformation which may be compatible with binding to a TNF receptor.

As a ligand that binds a Tumor Necrosis Factor Receptor, a portion of Ztnf11 may also dissociate from the cell and form a soluble ligand. For example, a protease cleavage site is located in the polypeptide sequence at about positions 84-85. Cleavage of Ztnf11 at this position will result in a soluble truncated Ztnf11 ligand comprising the amino acid sequence of residue 86 (Gln) to residue 309 of SEQ ID NO:2, or the amino acid as shown in SEQ ID NO: 6. Another possible cleavage site is located at about position 114-115. Cleavage of Ztnf11 at this position will result in a soluble truncated Ztnf11 ligand comprising the amino acid sequence of residue 116 (Gly) to 309 of SEQ ID NO:2, or the amino acid as shown in SEQ ID NO:7. Cleavage of Ztnf11 at this position can also result in a soluble truncated Ztnf11 ligand comprising the amino acid sequence of residue 115 (Arg) to 309 of SEQ ID NO:2, or the amino acid as shown in SEQ ID NO:7. Another cleavage site is located at about positions 77-78. Cleavage of Ztnf11 at this position will result in a soluble truncated Ztnf11 ligand comprising the amino acid sequence of residue 79 (Gln) to residue 309 of SEQ ID NO:2, or the amino acid as shown in SEQ ID NO: 8. Another possible soluble ztnf11 protein is a result of cleavages at about position 116 of SEQ ID NO:2 and position 267 or 272 of SEQ ID NO:2. Cleavage of Ztnf11 at this position will result in a soluble truncated Ztnf11 ligand comprising the amino acid sequence of residue 116 (Gly) to 267 (Met) or 272 (Arg) of SEQ ID NO:2. Polypeptide sequences for these soluble proteins are shown in SEQ ID NOs: 13 and 14, respectively. An additional cleavage at about residue 135 may result in a soluble polypeptide from residue 136 to residue 309 of SEQ ID NO:2.

As an additional example of a soluble ligand, Ztnf11 may be cleaved intracellularly and produced by the cell secretion pathway, not through cleavage of a membrane bound form. The TNF ligand, APRIL is expressed and processed in such a manner. As a soluble ligand, the polypeptides of SEQ ID NOs:6, 7, 8, 13, and 14, as well as the extracellular domain from amino acid 51 to amino acid 309, can be active at sites distant from their expression. Another example of a soluble ligand is the soluble ztnf11×2 polypeptide of SEQ ID NO:16. This polypeptide may be an inhibitor of the soluble forms of ztnf11, i.e., SEQ ID NOs: 6, 7, 8, 13, and 14. Other cleavage locations are possible between amino acid residues 51 and 130 of SEQ ID NO:2.

TNF ligands and TNF receptors are useful clinically to regulate autoimmune diseases, hematopoeisis, inflammation, cellular deficiencies, abnormal cellular proliferation, apoptosis, and cancers. For example, TNF ligands, such as TNFa, Apo2L/TRAIL, and BAFF, and the TNF receptors, such as TNF-R1, OPG 9, TACI-Fc 10, and BAFF-R 11 are being investigated in human clinical trials, or are already being marketed.

In addition to the TNF receptors for which a corresponding TNF ligand is known, there are several “orphan” TNF receptors for which a TNF ligand has not been shown to bind. These include, for example, TROY, RELT, DR6, and pMK61. DR6 contains a death domain and induces apoptosis. Its expression profile includes several lymphoid tissues, and is elevated in prostate/breast cancer. See Pan, G. et al. FEBS Letters 431: 351-356 (1998). DR6 and its corresponding ligand may play a role in T cell proliferation T helper differentiation, and in B cell expansion and humoral immune responses. See Liu, J. et al. Immunity 15: 23-34 (2001); Schmidt, C. S. et al. J. Exp. Med. 197: 51-62 (2003); and Zhao, H. et al. J. Exp. Med. 194: 1441-1448 (2001). The expression pattern of TROY, an EDA-R like receptor, appears to be broad, and includes expression in late developmental stages of the embryo as well as in the immune system. See Kojima, T. et al. J. Biol. Chem. 275: 20742-20747 (2000). RELT (receptor expressed in lymphoid tissues) is lymphoid-specific, and has been shown to co-stimulate T cell proliferation w/CD3. See Sica, G. L. et al. Blood 97: 2702-2707 (2001). RELT-Fc-biotin also binds PHA/ionomycin activated CD3+ cells by flow. The TNF receptor, pMK61, is expressed in peripheral lymphoid organs. IFN-g enhances pMK61-Fc binding to U937 and Jurkat, and pMK61-Fc inhibits Ig production in primary splenocytes. Ztnf11 may be a ligand that binds to a TNF receptor for which a corresponding ligand is known. Ztnf11 may also be a ligand for an “orphan” TNF receptor.

Analysis of the tissue distribution of Ztnf11 can be performed by the Northern blotting technique using Human Multiple Tissue and Master Dot Blots. Such blots are commercially available (Clontech, Palo Alto, Calif.) and can be probed by methods known to one skilled in the art. Also see, for example, Wu W. et al., Methods in Gene Biotechnology, CRC Press LLC, 1997. Additionally, portions of the polynucleotides of the present invention can be identified by querying sequence databases and identifying the tissues from which the sequences are derived. Portions of the polynucleotides of the present invention have been identified in testis, germ cell, and brain libraries, as well as from a library made from a pool of lung, testis, and B-cells.

The Ztnf11 gene as represented by (SEQ ID NO:1) is located on chromosome 17p13.1, and is located six genes upstream (about 150 kilobases) from other TNF ligands, Tweak and APRIL. Often genes from the same protein family are located near each other on the same chromosome.

The present invention also provides polynucleotide molecules, including DNA and RNA molecules, that encode the Ztnf11 polypeptides disclosed herein. Those skilled in the art will readily recognize that, in view of the degeneracy of the genetic code, considerable sequence variation is possible among these polynucleotide molecules. SEQ ID NO:3 is a degenerate DNA sequence that encompasses all DNAs that encode the Ztnf11 polypeptide of SEQ ID NO:2. Those skilled in the art will recognize that the degenerate sequence of SEQ ID NO:3 also provides all RNA sequences encoding SEQ ID NO:2 by substituting U (uracil) for T (thymine). Thus, Ztnf11 polypeptide-encoding polynucleotides comprising nucleotide 1 to nucleotide 927 of SEQ ID NO:3 and their RNA equivalents are contemplated by the present invention.

Table 1 sets forth the one-letter codes used within SEQ ID NO:3 to denote degenerate nucleotide positions. “Resolutions” are the nucleotides denoted by a code letter. “Complement” indicates the code for the complementary nucleotide(s). For example, the code Y denotes either C (cytosine) or T, and its complement R denotes A (adenine) or G (guanine), A being complementary to T, and G being complementary to C.

TABLE 1

| Nucleotide | Resolution | Nucleotide | Complement |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:3, encompassing all possible codons for a given amino acid, are set forth in Table 2.

TABLE 2

| | One | | |
|---|---|---|---|
| Amino Letter | | Degenerate | |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. Biol. 6:315-24, 1996; Wain-Hobson, et al., Gene 13:355-64, 1981; Grosjean and Fiers, Gene 18:199-209, 1982; Holm, Nuc. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequence disclosed in SEQ ID NO:3 serves as a template for optimizing expression of polynucleotides in various cell types and species commonly used in the art and disclosed herein. Sequences containing preferential codons can be tested and optimized for expression in various species, and tested for functionality as disclosed herein.

Within preferred embodiments of the invention, isolated polynucleotides will hybridize to similar sized regions of SEQ ID NO:1, or to a sequence complementary thereto, under stringent conditions. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typical stringent conditions are those in which the salt concentration is up to about 0.03 M at pH 7 and the temperature is at least about 60° C. As previously noted, the isolated polynucleotides of the present invention include DNA and RNA. Methods for isolating DNA and RNA are well known in the art. It is generally preferred to isolate RNA from testis, although DNA can also be prepared using RNA from other tissues or isolated as genomic DNA. Total RNA can be prepared using guanidine HCl extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. Polynucleotides encoding Ztnf11 polypeptides are then identified and isolated by, for example, hybridization or PCR.

Those skilled in the art will recognize that the sequence disclosed in SEQ ID NO:1 represents a single allele of the human Ztnf11 gene, and that allelic variation and alternative splicing are expected to exist. Allelic variants of the DNA sequence shown in SEQ ID NO:1, including those containing silent mutations and those in which mutations result in amino acid sequence changes, are within the scope of the present invention, as are proteins which are allelic variants of SEQ ID NO:2. cDNAs generated from alternatively spliced mRNAs, which retain the properties of the Ztnf11 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

The present invention further provides counterpart ligands and polynucleotides from other species (“species orthologs”). These species include, but are not limited to mammalian, avian, amphibian, reptile, fish, insect and other vertebrate and invertebrate species. Of particular interest are Ztnf11 ligand polypeptides from other mammalian species, including murine, porcine, ovine, bovine, canine, feline, equine, and other primate ligands. Species orthologs of human Ztnf11 can be cloned using information and compositions provided by the present invention in combination with conventional cloning techniques. For example, a cDNA can be cloned using mRNA obtained from a tissue or cell type that expresses the ligand. Suitable sources of mRNA can be identified by probing Northern blots with probes designed from the sequences disclosed herein. A library is then prepared from mRNA of a positive tissue or cell line. A Ztnf11-encoding cDNA can then be isolated by a variety of methods, such as by probing with a complete or partial human cDNA or with one or more sets of degenerate probes based on the disclosed sequence. A cDNA can also be cloned using the polymerase chain reaction (PCR) (Mullis, U.S. Pat. No. 4,683,202), using primers designed from the sequences disclosed herein. Within an additional method, the cDNA library can be used to transform or transfect host cells, and expression of the cDNA of interest can be detected with an antibody to Ztnf11. Similar techniques can also be applied to the isolation of genomic clones.

Alternate species polypeptides of Ztnf11 may have importance therapeutically. It has been demonstrated that in some cases use of a non-native protein, i.e., protein from a different species, can be more potent than the native protein. For example, salmon calcitonin has been shown to be considerably more effective in arresting bone resorption than human forms of calcitonin. There are several hypotheses as to why salmon calcitonin is more potent than human calcitonin in treatment of osteoporosis. These hypotheses include: 1) salmon calcitonin is more resistant to degradation; 2) salmon calcitonin has a lower metabolic clearance rate (MCR); and 3) salmon calcitonin may have a slightly different conformation, resulting in a higher affinity for bone receptor sites. Another example is found in the β-endorphin family (Ho et al., Int. J. Peptide Protein Res. 29:521-4, 1987). Studies have demonstrated that the peripheral opioid activity of camel, horse, turkey and ostrich β-endorphins is greater than that of human β-endorphins when isolated guinea pig ileum was electrostimulated and contractions were measured. Vas deferens from rat, mouse and rabbit were assayed as well. In the rat vas deferens model, camel and horse β-endorphins showed the highest relative potency. Synthesized rat relaxin was as active as human and porcine relaxin in the mouse symphysis pubis assay (Bullesbach and Schwabe, Eur. J. Biochem. 241:533-7, 1996). Thus, the mouse Ztnf11 molecules of the present invention may have higher potency than the human endogenous molecule in human cells, tissues and recipients. The polynucleotide and polypeptide sequences for the mouse Ztnf11 are provided in SEQ ID NOs: 9 and 10, respectively.

The present invention also provides isolated ligand polypeptides that are substantially homologous to the ligand polypeptide of SEQ ID NO:2 and its species orthologs. In a preferred form, the isolated protein or polypeptide is substantially free of other proteins or polypeptides, particularly other proteins or polypeptides of animal origin. It is preferred to provide the proteins or polypeptides in a highly purified form, i.e. greater than 95% pure, more preferably greater than 99% pure. The term “substantially homologous” is used herein to denote proteins or polypeptides having 50%, preferably 60%, more preferably at least 80%, sequence identity to the sequence shown in SEQ ID NO:2 or its species orthologs. Such proteins or polypeptides will more preferably be at least 90% identical, and most preferably 95% or more identical to SEQ ID NO:2 or its species orthologs or paralogs. Percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 3 (amino acids are indicated by the standard one-letter codes). The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Sequence identity of polynucleotide molecules is determined by similar methods using a ratio as disclosed above.

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Substantially homologous proteins and polypeptides are characterized as having one or more amino acid substitutions, deletions or additions. These changes are preferably of a minor nature, that is conservative amino acid substitutions (see Table 4) and other substitutions that do not significantly affect the folding or activity of the protein or polypeptide; small deletions, typically of one to about 30 amino acids; and small amino- or carboxyl-terminal extensions, such as an amino-terminal methionine residue, a small linker peptide of up to about 20-25 residues, or an affinity tag. The present invention thus includes polypeptides of from 184 to 1000 amino acid residues that comprise a sequence that is at least 60%, preferably at least 80%, and more preferably 90% and even more preferably 95% or more identical to the corresponding region of SEQ ID NO:2. Polypeptides comprising affinity tags can further comprise a proteolytic cleavage site between the Ztnf11 polypeptide and the affinity tag. Preferred such sites include thrombin cleavage sites and factor Xa cleavage sites.

TABLE 4
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of Ztnf11 polypeptides of the present invention. A limited number of non-conservative amino acids, amino acids that are not encoded by the genetic code, and unnatural amino acids may be substituted for Ztnf11 polypeptide amino acid residues. The proteins of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for Ztnf11 amino acid residues.

Essential amino acids in the Ztnf11 polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related cystatin family members.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed Ztnf11 DNA and polypeptide sequences can be generated through DNA shuffling as disclosed by Stemmer, Nature 370:389-91, 1994, Stemmer, Proc. Natl. Acad. Sci. USA 91:10747-51, 1994 and WIPO Publication WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed above can be combined with high-throughput screening methods to detect activity of cloned, mutagenized ligands. Mutagenized DNA molecules that encode active ligands or portions thereof (e.g., receptor-binding fragments) can be recovered from the host cells and rapidly sequenced using modern equipment. These methods allow the rapid determination of the importance of individual amino acid residues in a polypeptide of interest, and can be applied to polypeptides of unknown structure.

Using the methods discussed above, one of ordinary skill in the art can identify and/or prepare a variety of polypeptides that are substantially homologous to the soluble ligands, or allelic variants thereof and retain the receptor-binding properties of the wild-type protein. Examples of the soluble ligands are listed above, and include polypeptides comprising the amino acid sequences of residue 115 to 309 of SEQ ID NO:2 (i.e., the polypeptide consisting of or comprising the amino acid sequence of SEQ ID NO:7), residue 86 to 309 of SEQ ID NO:2 (i.e., the polypeptide consisting of or comprising the amino acid sequence of SEQ ID NO:6), residue 116 to 309 of SEQ ID NO:2 (i.e., the polypeptide consisting of or comprising the amino acid sequence of SEQ ID NO:7), residue 51 to 309 of SEQ ID NO:2 (i.e., the polypeptide consisting of or comprising the amino acid sequence of SEQ ID NO:17), residue 79 to 309 of SEQ ID NO:2 (i.e., the polypeptide consisting of or comprising the amino acid sequence of SEQ ID NO:8), residue 116 to 267 of SEQ ID NO:2 (i.e., the polypeptide consisting of or comprising the amino acid sequence of SEQ ID NO:13), and residue 116 to 272 of SEQ ID NO:2 (i.e., the polypeptide consisting of or comprising the amino acid sequence of SEQ ID NO:14). Such polypeptides may include additional amino acids from the transmembrane domain, linker and/or cytoplasmic domain; affinity tags; and the like. Such polypeptides may also include additional polypeptide segments as generally disclosed above.

The ligand polypeptides of the present invention, including full-length ligand polypeptides, ligand fragments (e.g., receptor-binding fragments), and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, Second Edition, Cold Spring Harbor, N.Y., 1989; and Ausubel et al., eds., Current Protocols in Molecular Biology, John Wiley and Sons, Inc., N.Y., 1987.

For any Ztnf11 polypeptide, including variants and fusion proteins, one of ordinary skill in the art can readily generate a fully degenerate polynucleotide sequence encoding that variant using the information set forth in Tables 1 and 2 above.

In general, a DNA sequence encoding a Ztnf11 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a Ztnf11 polypeptide into the secretory pathway of a host cell, a secretory signal sequence (also known as a signal sequence, leader sequence, prepro sequence or pre sequence) is provided in the expression vector. The secretory signal sequence may be derived from another secreted protein (e.g., t-PA) or synthesized de novo. The secretory signal sequence is joined to the Ztnf11 DNA sequence in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

Since multimeric complexes of the TNF ligand and TNF receptor families are known to be biologically active, it may be useful to prepare fusion proteins of Ztnf11 with another TNF ligand. For example, APRIL and BAFF can form heterotrimeric ligands. Thus, Ztnf11 may form multimers, including but not limited to dimers, trimers, heterodimers and hererotrimers with another TNF ligand. Such ligand may includes for example, APRIL, Tweak, Lt-Beta, ztnf4, CD-27 ligand, and RANK-L. The fusion protein can be prepared with the Ztnf11 polynucleotide sequence, or a portion thereof, at the amino terminal followed by the carboxyl terminal of the other TNF ligand. Similarly, Ztnf11 polypeptides, or fragments thereof, can be used as an agonist of APRIL, Tweak, Lt-Beta, ztnf4, CD-27 ligand, and/or RANK-L activity by binding the corresponding TNF receptor. For the example of RANK-L, binding of the TNF receptor, RANK will result in stimulating osteoclast activity. (See Li, J. et al., P.N.A.S. 1566-1571, 2000.) Alternatively, these polypeptides can be used as an inhibitor of APRIL, Tweak, Lt-Beta, ztnf4, CD-27 ligand, and/or RANK-L activity by binding the corresponding TNF receptor, but failing to result in an intracellular signal.

As discussed above, it is likely that Ztnf11 polypeptides will form a trimer to facilitate receptor binding. Of note, however, it may not be necessary for TNF receptor polypeptides to form a trimeric complex. Bazzoni (Bazzoni, F. et al., P.N.A.S.92: 5376-5380, 1995) have shown that for some TNF receptors, dimerization (rather than trimerization or higher-order multimerization) was sufficient. Thus, Ztnf11 polypeptides may be useful as dimers, timers, multimers, or a combination thereof. For an example of how to make ztnf11 trimers, see, for example, Wu, X. et al., Mol. Ther.3:368-374, 2001.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981; Graham and Van der Eb, Virology 52:456, 1973), electroporation (Neumann et al., EMBO J. 1:841-45, 1982), DEAE-dextran mediated transfection (Ausubel et al., ibid), and liposome-mediated transfection (Hawley-Nelson et al., Focus 15:73, 1993; Ciccarone et al., Focus 15:80, 1993). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the U-937 (ATCC No. CRL-1593.2), HL-60 (ATCC No. CCL-240), COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g., CHO-K1; ATCC No. CCL 61) cell lines. Additional suitable cell lines are known in the art and available from public depositories such as the American Type Culture Collection, Rockville, Md. In general, strong transcription promoters are preferred, such as promoters from SV40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems may also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g., hygromycin resistance, multi-drug resistance, puromycin acetyltransferase) can also be used. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). DNA encoding the Ztnf11 polypeptide is inserted into the baculoviral genome in place of the AcNPV polyhedrin gene coding sequence by one of two methods. The first is the traditional method of homologous DNA recombination between wild-type AcNPV and a transfer vector containing the Ztnf11 flanked by AcNPV sequences. Suitable insect cells, e.g. SF9 cells, are infected with wild-type AcNPV and transfected with a transfer vector comprising a Ztnf11 polynucleotide operably linked to an AcNPV polyhedrin gene promoter, terminator, and flanking sequences. See, King and Possee, The Baculovirus Expression System: A Laboratory Guide, London, Chapman & Hall; O'Reilly et al., Baculovirus Expression Vectors: A Laboratory Manual, New York, Oxford University Press., 1994; and, Richardson (Ed.), Baculovirus Expression Protocols. Methods in Molecular Biology, Totowa, N.J., Humana Press, 1995. Natural recombination within an insect cell will result in a recombinant baculovirus which contains Ztnf11 driven by the polyhedrin promoter. Recombinant viral stocks are made by methods commonly used in the art.

The second method of making recombinant baculovirus utilizes a transposon-based system described by Luckow et al. (J. Virol. 67:4566-79, 1993). This system is sold in the Bac-to-Bac kit (Life Technologies, Rockville, Md.). This system utilizes a transfer vector, pFastBac1™ (Life Technologies) containing a Tn7 transposon to move the DNA encoding the Ztnf11 polypeptide into a baculovirus genome maintained in E. coli as a large plasmid called a “bacmid.” The pFastBac1™ transfer vector utilizes the AcNPV polyhedrin promoter to drive the expression of the gene of interest, in this case Ztnf11. However, pFastBac1™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins and Possee, J. Gen. Virol. 71:971-6, 1990; Bonning et al., J. Gen. Virol. 75:1551-6, 1994; and, Chazenbalk and Rapoport, J. Biol. Chem. 270:1543-9, 1995. In such transfer vector constructs, a short or long version of the basic protein promoter can be used. Moreover, transfer vectors can be constructed which replace the native Ztnf11 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native Ztnf11 secretory signal sequence. In addition, transfer vectors can include an in-frame fusion with DNA encoding an epitope tag at the C— or N-terminus of the expressed Ztnf11 polypeptide, for example, a Glu-Glu epitope tag (Grussenmeyer et al., Proc. Natl. Acad. Sci. 82:7952-4, 1985) or FLAG tag. Using a technique known in the art, a transfer vector containing Ztnf11 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses Ztnf11 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435). Commercially available serum-free media are used to grow and maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. The recombinant virus-infected cells typically produce the recombinant Ztnf11 polypeptide at 12-72 hours post-infection and secrete it with varying efficiency into the medium. The culture is usually harvested 48 hours post-infection. Centrifugation is used to separate the cells from the medium (supernatant). The supernatant containing the Ztnf11 polypeptide is filtered through micropore filters, usually 0.45 μm pore size. Procedures used are generally described in available laboratory manuals (King and Possee, ibid.; O'Reilly et al., ibid.; Richardson, ibid.). Subsequent purification of the Ztnf11 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in S. cerevisiae is the POTI vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, P. pastoris, P. methanolica, P. guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publications WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A preferred selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRB1) are preferred. Electroporation is used to facilitate the introduction of a plasmid containing DNA encoding a polypeptide of interest into P. methanolica cells. It is preferred to transform P. methanolica cells by electroporation using an exponentially decaying, pulsed electric field having a field strength of from 2.5 to 4.5 kV/cm, preferably about 3.75 kV/cm, and a time constant (T) of from 1 to 40 milliseconds, most preferably about 20 milliseconds.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a Ztnf11 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% Bacto™ Peptone (Difco Laboratories, Detroit, Mich.), 1% Bacto™ yeast extract (Difco Laboratories), 0.004% adenine and 0.006% L-leucine).

Expressed recombinant Ztnf11 polypeptides (or chimeric Ztnf11 polypeptides) can be purified using fractionation and/or conventional purification methods and media. Ammonium sulfate precipitation and acid or chaotrope extraction may be used for fractionation of samples. Exemplary purification steps may include hydroxyapatite, size exclusion, FPLC and reverse-phase high performance liquid chromatography. Suitable anion exchange media include derivatized dextrans, agarose, cellulose, polyacrylamide, specialty silicas, and the like. PEI, DEAE, QAE and Q derivatives are preferred, with DEAE Fast-Flow Sepharose (Pharmacia, Piscataway, N.J.) being particularly preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, 1988.

The polypeptides of the present invention can be isolated by exploitation of their physical properties. For example, immobilized metal ion adsorption (IMAC) chromatography can be used to purify histidine-rich proteins, including those having His-tags. Briefly, a gel is first charged with divalent metal ions to form a chelate (E. Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp.529-39). Within additional embodiments of the invention, a fusion of the polypeptide of interest and an affinity tag (e.g., Glu-Glu, FLAG, maltose-binding protein, an immunoglobulin domain) may be constructed to facilitate purification.

Protein refolding (and optionally reoxidation) procedures may be advantageously used. It is preferred to purify the protein to >80% purity, more preferably to >90% purity, even more preferably >95%, and particularly preferred is a pharmaceutically pure state, that is greater than 99.9% pure with respect to contaminating macromolecules, particularly other proteins and nucleic acids, and free of infectious and pyrogenic agents. Preferably, a purified protein is substantially free of other proteins, particularly other proteins of animal origin.

Ztnf11 polypeptides or fragments thereof may also be prepared through chemical synthesis. Ztnf11 polypeptides may be monomers or multimers;

glycosylated or non-glycosylated; pegylated or non-pegylated; and may or may not include an initial methionine amino acid residue.

The invention also provides soluble Ztnf11 ligands. The soluble ligand can include polypeptides comprising the amino acid sequences of residue 115 to 309 of SEQ ID NO:2 (i.e., the polypeptide consisting of or comprising the amino acid sequence of SEQ ID NO:7), residue 86 to 309 of SEQ ID NO:2 (i.e., the polypeptide consisting of or comprising the amino acid sequence of SEQ ID NO:6), residue 116 to 309 of SEQ ID NO:2 (i.e., the polypeptide consisting of or comprising the amino acid sequence of SEQ ID NO:7), residue 51 to 309 of SEQ ID NO:2 (i.e., the polypeptide consisting of or comprising the amino acid sequence of SEQ ID NO:17), residue 79 to 309 of SEQ ID NO:2 (i.e., the polypeptide consisting of or comprising the amino acid sequence of SEQ ID NO:8), residue 116 to 267 of SEQ ID NO:2 (i.e., the polypeptide consisting of or comprising the amino acid sequence of SEQ ID NO:13), and residue 116 to 272 of SEQ ID NO:2 (i.e., the polypeptide consisting of or comprising the amino acid sequence of SEQ ID NO: 14), or the corresponding region of a non-human ligand. Such soluble polypeptides can be used to form fusion proteins with human Ig, as His-tagged proteins or as N— or C-terminal FLAG™-tagged (Hopp et al., Biotechnology 6:1204-10, 1988) or Glu-Glu tagged proteins. It is preferred that the extracellular receptor-binding domain polypeptides be prepared in a form substantially free of transmembrane and intracellular polypeptide segments. For example, the N-terminus of the receptor-binding domain may be at amino acid residue 51, 86, 115, 116, or 79 of SEQ ID NO:2, or at the corresponding region of an allelic variant or a non-human ligand. To direct the export of the soluble ligand from the host cell, the truncated ligand DNA is linked to a second DNA segment encoding a secretory peptide, such as a t-PA secretory peptide. To facilitate purification of the secreted soluble ligand, a C-terminal extension, such as a poly-histidine tag, substance P, Flag™ peptide (Hopp et al., ibid; available from Eastman Kodak Co., New Haven, Conn.) or another polypeptide or protein for which an antibody or other specific binding agent is available, can be fused to the soluble ligand polypeptide at either the N or C terminus.

In an alternative approach, an extracellular receptor-binding domain can be expressed as a fusion with immunoglobulin heavy chain constant regions, typically an F_{C} fragment, which contains two constant region domains and a hinge region, but lacks the variable region. Such fusions are typically secreted as multimeric molecules, wherein the Fc portions are disulfide bonded to each other and two ligand polypeptides are arrayed in close proximity to each other. Fusions of this type can be used to affinity purify the cognate receptor from solution, as an in vitro assay tool, and to block signals in vitro by specifically titrating out or blocking endogenous ligand. To purify soluble receptor, a Ztnf11-Ig fusion protein (chimera) is added to a sample containing the soluble receptor under conditions that facilitate receptor-ligand binding (typically near-physiological temperature, pH, and ionic strength). The chimera-receptor complex is then separated from the mixture using protein A, which is immobilized on a solid support (e.g., insoluble resin beads). The receptor is then eluted using conventional chemical techniques, such as with a salt or pH gradient. In the alternative, the chimera itself can be bound to a solid support, with binding and elution carried out as above. Collected fractions can be re-fractionated until the desired level of purity is reached. For use in assays, the chimeras are bound to a support via the Fc region and used in an ELISA format. Conversely, soluble TNF receptor-Ig fusion proteins may be made using TNF receptors for which a ligand has not been identified. Soluble Ztnf11 is then mixed with a receptor fusion protein and binding is assayed as described above. The chimeras may be used in vivo as an anti-inflammatory, in the inhibition of autoimmune processes, for inhibition of antigen in humoral and cellular immunity and for immunosuppression in graft and organ transplants. The chimeras may also be used to stimulate lymphocyte development, such as during bone marrow transplantation and as therapy for some cancers.

An assay system that uses a ligand-binding receptor (or an antibody, one member of a complement/ anti-complement pair) or a binding fragment thereof, and a commercially available biosensor instrument (BIAcore™, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding.

Ztnf11 polynucleotides and/or polypeptides may be useful for regulating the proliferation and stimulation of a wide variety of TNF receptor-bearing cells, such as T cells, lymphocytes, peripheral blood mononuclear cells, polymorphonuclear leukocytes, fibroblasts, hematopoietic cells and a variety of cells in testis tissue. Other tumor necrosis factors, such as gp39 and TNFβ also stimulate B cell proliferation. Ztnf11 polypeptides will also find use in mediating metabolic or physiological processes in vivo. Proliferation and differentiation can be measured in vitro using cultured cells. Bioassays and ELISAs are available to measure cellular response to Ztnf11, in particular are those which measure changes in cytokine production as a measure of cellular response (see for example, Current Protocols in Immunology ed. John E. Coligan et al., NIH, 1996). Assays to measure other cellular responses, including antibody isotype, monocyte activation, NK cell formation, antigen presenting cell function, apoptosis.

A variety of assays are also available to measure bone formation and resorption. These assays measure, for example, serum calcium levels, osteoclast size and number, osteoblast size and number, ostenopenia induced by estrogen deficiency, cancellous bone volumes of the distal femur (mouse), cartilaginous growth plates, and chondrocyte formation and differentiation. The Ztnf11 polypeptides of the present invention can be measured in any of these assay, as well as additional assays dislcosed herein, and assays that are readily known to one of skill in the art.

In another embodiment, the cell activation is determined by measuring proliferation using ³H-thymidine uptake (Crowley et al., J. Immunol. Meth. 133:55-66, 1990). Alternatively, cell activation can be measured by the production of cytokines, such as IL-2, or by determining the presence of cell-specific activation markers. Cytokine production can be assayed by testing the ability of the Ztnf11 and cell culture supernatant to stimulate growth of cytokine-dependent cells. Cell specific activation markers may be detected using antibodies specific for such markers.

In vitro and in vivo response to Ztnf11 can also be measured using cultured cells or by administering molecules of the claimed invention to the appropriate animal model. One in vivo approach for assaying proteins of the present invention involves viral delivery systems. Exemplary viruses for this purpose include adenovirus, herpesvirus, vaccinia virus and adeno-associated virus (AAV). Adenovirus, a double-stranded DNA virus, is currently the best studied gene transfer vector for delivery of heterologous nucleic acid (for a review, see Becker et al., Meth. Cell Biol. 43:161-89, 1994; and Douglas and Curiel, Science & Medicine 4:44-53, 1997). The adenovirus system offers several advantages: adenovirus can (i) accommodate relatively large DNA inserts; (ii) be grown to high-titer; (iii) infect a broad range of mammalian cell types; and (iv) be used with a large number of available vectors containing different promoters. Also, because adenoviruses are stable in the bloodstream, they can be administered by intravenous injection. Some disadvantages (especially for gene therapy) associated with adenovirus gene delivery include: (i) very low efficiency integration into the host genome; (ii) existence in primarily episomal form; and (iii) the host immune response to the administered virus, precluding readministration of the adenoviral vector.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a signal sequence is present, secrete) the heterologous protein. Secreted proteins will enter the circulation in the highly vascularized liver, and effects on the infected animal can be determined.

The adenovirus system can also be used for protein production in vitro.

By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293S cells can be grown in suspension culture at relatively high cell density to produce significant amounts of protein (see Garnier et al., Cytotechnol. 15:145-55, 1994). With either protocol, an expressed, secreted heterologous protein can be repeatedly isolated from the cell culture supernatant. Within the infected 293S cell production protocol, non-secreted proteins may also be effectively obtained.

Well established animal models are available to test in vivo efficacy of Ztnf11 polypeptides for certain disease states. In particular, Ztnf11 polypeptides can be tested in vivo in a number of animal models of autoimmune disease, such as the NOD mice, a spontaneous model system for insulin-dependent diabetes mellitus (IDDM), to study induction of non-responsiveness in the animal model. Administration of Ztnf11 polypeptides prior to or after onset of disease can be monitored by assay of urine glucose levels in the NOD mouse. Alternatively, induced models of autoimmune disease, such as experimental allergic encephalitis (EAE), can be administered Ztnf11 polypeptides. Administration in a preventive or intervention mode can be followed by monitoring the clinical symptoms of EAE.

Ztnf11 polypeptides can also be used to prepare antibodies that specifically bind to Ztnf11 epitopes, peptides or polypeptides. Methods for preparing polyclonal and monoclonal antibodies are well known in the art (see, for example, Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982). As would be evident to one of ordinary skill in the art, polyclonal antibodies can be generated from a variety of warm-blooded animals, such as horses, cows, goats, sheep, dogs, chickens, rabbits, mice, and rats.

The immunogenicity of a Ztnf11 polypeptide may be increased through the use of an adjuvant, such as alum (aluminum hydroxide) or Freund's complete or incomplete adjuvant. Polypeptides useful for immunization also include fusion polypeptides, such as fusions of Ztnf11 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments thereof, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting only non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. Humanized monoclonal antibodies directed against Ztnf11 polypeptides could be used as a protein therapeutic, in particular for use as an immunotherapy. Alternative techniques for generating or selecting antibodies useful herein include in vitro exposure of testis tissue to Ztnf11 protein or peptide, and selection of antibody display libraries in phage or similar vectors (for instance, through use of immobilized or labeled Ztnf11 protein or peptide).

Antibodies are defined to be specifically binding if they bind to a Ztnf11 polypeptide with a binding affinity (Ka) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art (for example, by Scatchard analysis).

A variety of assays known to those skilled in the art can be utilized to detect antibodies which specifically bind to Ztnf11 proteins or peptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, ELISA, dot blot or Western blot assay, inhibition or competition assay, and sandwich assay. In addition, antibodies can be screened for binding to wild-type versus mutant Ztnf11 protein or peptide.

Antibodies to Ztnf11 may be used for immunohistochemical tagging of cells that express human Ztnf11, for example, to use in a diagnostic assays; for isolating Ztnf11 by affinity purification; for screening expression libraries; for generating anti-idiotypic antibodies; and as neutralizing antibodies or as antagonists to block Ztnf11 in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications.

Antibodies to soluble Ztnf11 polypeptides, including ligands comprising the amino acid sequences of residue 115 to 309 of SEQ ID NO:2 (the polypeptide having the amino acid sequence of SEQ ID NO:7), residue 86 to 309 of SEQ ID NO:2 (the polypeptide having the amino acid sequence of SEQ ID NO:6), residue 116 to 309 of SEQ ID NO:2 (the polypeptide having the amino acid sequence of SEQ ID NO:7), residue 51 to 309 of SEQ ID NO:2 (the polypeptide having the amino acid sequence of SEQ ID NO:17), residue 79 to 309 of SEQ ID NO:2 (the polypeptide having the amino acid sequence of SEQ ID NO:8), residue 116 to 267 of SEQ ID NO:2 (the polypeptide having the amino acid sequence of SEQ ID NO:13), and residue 116 to 272 of SEQ ID NO:2 (the polypeptide having the amino acid sequence of SEQ ID NO:14) can also be prepared. Such soluble polypeptides can also be His, Glu-Glu or FLAG tagged. Alternatively such polypeptides form a fusion protein with human Ig. In particular, antiserum containing anti-polypeptide antibodies directed to His-, Glu-Glu- or FLAG-tagged soluble Ztnf11 can be used in analysis of tissue distribution of Ztnf11 or receptors that bind Ztnf11 by immunohistochemistry on human or primate tissue. These soluble Ztnf11 polypeptides can also be used to immunize mice in order to produce monoclonal antibodies to a soluble human Ztnf11 polypeptide. Monoclonal antibodies to a soluble human Ztnf11 polypeptide can be used to analyze hematopoietic cell distribution using methods known in the art, such as three color fluorescence immunocytometry. Monoclonal antibodies to a soluble human Ztnf11 polypeptide can also be used to mimic ligand/receptor coupling, resulting in activation or inactivation of the ligand/receptor pair. For instance, it has been demonstrated that cross-linking anti-soluble GP39 monoclonal antibodies inhibits signal from T cells to B cells (Noelle et al., Proc. Natl. Acad. Sci. USA 89:6650, 1992). Monoclonal antibodies to Ztnf11 can be used to determine the distribution, regulation and biological interaction of the Ztnf11 receptor/Ztnf11 ligand pair on specific cell lineages identified by tissue distribution studies, in particular, T cell lineages. Antibodies to Ztnf11 can also be used to detect secreted, soluble Ztnf11 in biological samples.

Antigenic epitope-bearing peptides and polypeptides contain at least four to ten amino acids, or at least ten to fifteen amino acids, or 15 to 30 amino acids of SEQ ID NO:2. Such epitope-bearing peptides and polypeptides can be produced by fragmenting an Ztnf11 polypeptide, or by chemical peptide synthesis, as described herein. Moreover, epitopes can be selected by phage display of random peptide libraries (see, for example, Lane and Stephen, Curr. Opin. Immunol. 5:268 (1993), and Cortese et al., Curr. Opin. Biotechnol. 7:616 (1996)). Standard methods for identifying epitopes and producing antibodies from small peptides that comprise an epitope are described, for example, by Mole, “Epitope Mapping,” in Methods in Molecular Biology, Vol. 10, Manson (ed.), pages 105-116 (The Humana Press, Inc. 1992), Price, “Production and Characterization of Synthetic Peptide-Derived Antibodies,” in Monoclonal Antibodies: Production, Engineering, and Clinical Application, Ritter and Ladyman (eds.), pages 60-84 (Cambridge University Press 1995), and Coligan et al. (eds.), Current Protocols in Immunology, pages 9.3.1-9.3.5 and pages 9.4.1-9.4.11 (John Wiley & Sons 1997).

Ztnf11 polypeptides can also be used to prepare antibodies that specifically bind to Ztnf11 epitopes, peptides or polypeptides. The Ztnf11 polypeptide or a fragment thereof serves as an antigen (immunogen) to inoculate an animal and elicit an immune response. One of skill in the art would recognize that antigenic, epitope-bearing polypeptides contain a sequence of at least 6, or at least 9, and at least 15 to about 30 contiguous amino acid residues of a Ztnf11 polypeptide (e.g., SEQ ID NO:2). Polypeptides comprising a larger portion of a Ztnf11 polypeptide, i.e., from 30 to 10 residues up to the entire length of the amino acid sequence are included. Antigens or immunogenic epitopes can also include attached tags, adjuvants and carriers, as described herein. Suitable antigens include the Ztnf11 polypeptides encoded by SEQ ID NO:2 from amino acid number 1 to amino acid number 309, or a contiguous 9 to 309 amino acid fragment thereof. Additional suitable antigens include the Ztnf11×2 polypeptides encoded by SEQ ID NO:15 from amino acid number 1 to amino acid number 121, or a contiguous 9 to 121 amino acid fragment thereof.

As an illustration, potential antigenic sites in Ztnf11 were identified using the Jameson-Wolf method, Jameson and Wolf, CABIOS 4:181, (1988), as implemented by the PROTEAN program (version 3.14) of LASERGENE (DNASTAR; Madison, Wis.). Default parameters were used in this analysis.

Suitable antigens include the peptides comprising amino acid sequences selected from the group consisting of: residue 65 to 88 of SEQ ID NO:2; residue 105 to 122 of SEQ ID NO:2; residue 128 to 139 of SEQ ID NO:2; residue 148 to 162 of SEQ ID NO:2; residue 168 to 178 of SEQ ID NO:2; residue 183 to 192 of SEQ ID NO:2; residue 210 to 232 of SEQ ID NO:2; residue 250 to 281 of SEQ ID NO:2; and residue 291 to 301 of SEQ ID NO:2. Hydrophilic peptides, such as those predicted by one of skill in the art from a hydrophobicity plot are also immunogenic. Ztnf11 hydrophilic peptides include peptides comprising amino acid sequences selected from the group consisting of: residue 75 to 88 of SEQ ID NO:2; residue 108 to 119 of SEQ ID NO:2; residue 131 to 139 of SEQ ID NO:2; residue 149 to 160 of SEQ ID NO:2; residue 169 to 174 of SEQ ID NO:2; residue 184 to 190 of SEQ ID NO:2; residue 209 to 233 of SEQ ID NO:2; residue 252 to 257 of SEQ ID NO:2; residue 265 to 276 of SEQ ID NO:2; and residue 293 to 301 of SEQ ID NO:2. Additionally, antigens can be generated to portions of the polypeptide which are likely to be on the surface of the folded protein. These antigens include: residue 5 to residue 13 of SEQ ID NO:2; residue 74 to 87 of SEQ ID NO:2; residue 107 to 117 of SEQ ID NO:2; residue 130 to 139 of SEQ ID NO:2; residue 149 to 161 of SEQ ID NO:2; residue 167 to 182 of SEQ ID NO:2; residue 184 to 190 of SEQ ID NO:2; residue 207 to 222 of SEQ ID NO:2; residue 203 to 238 of SEQ ID NO:2; residue 251 to 257 of SEQ ID NO:2; residue 263 to 277 of SEQ ID NO:2; and residue 292 to 302 of SEQ ID NO:2. Antibodies from an immune response generated by inoculation of an animal with these antigens can be isolated and purified as described herein. Methods for preparing and isolating polyclonal and monoclonal antibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982.

Ztnf11 ligand polypeptides and soluble Ztnf11 ligands may be used to identify and characterize receptors in the TNFR family. Ztnf11 may bind one of the known members of the TNFR family, such as TNF and lymphotoxin-α bind to the TNF receptor. Proteins and peptides of the present invention can be immobilized on a column and membrane preparations run over the column (Immobilized Affinity Ligand Techniques, Hermanson et al., eds., Academic Press, San Diego, Calif., 1992, 195-202). Proteins and peptides can also be radiolabeled (Methods in Enzymol., vol. 182, “Guide to Protein Purification”, M. Deutscher, ed., Acad. Press, San Diego, 1990, 721-37) or photoaffinity labeled (Brunner et al., Ann. Rev. Biochem. 62:483-514, 1993 and Fedan et al., Biochem. Pharmacol. 33:1167-80, 1984) and specific cell-surface proteins can be identified. The soluble ligand is useful in studying the distribution of receptors on tissues or specific cell lineages, and to provide insight into receptor/ligand biology. Application may also be made of the specificity of TNF ligands for their receptor as a mechanism by which to destroy receptor-bearing target cells. For example, toxic compounds may be coupled to Ztnf11 ligands, in particular to soluble ligands (Mesri et al., J. Biol. Chem. 268:4853-62, 1993). Examples of toxic compounds would include radiopharmaceuticals that inactivate target cells; chemotherapeutic agents such as doxorubicin, daunorubicin, methotrexate, and cytoxan; toxins, such as ricin, diphtheria, Pseudomonas exotoxin A and abrin; and antibodies to cytotoxic T-cell surface molecules.

As a TNF ligand, Ztnf11 will be useful to treat hematopoeisis, inflammation, cellular deficiencies, abnormal cellular proliferation, apoptosis,. cancers, and includes disorders, acute and chronic, of the immune and/inflammatory response. Inflammation normally is a localized, protective response to trauma or microbial invasion that destroys, dilutes, or walls-off the injurious agent and the injured tissue. Diseases characterized by inflammation are significant causes of morbidity and mortality in humans. While inflammation commonly occurs as a defensive response to invasion of the host by foreign material, it is also triggered by a response to mechanical trauma, toxins, and neoplasia. Excessive inflammation caused by abnormal recognition of host tissue as foreign, or prolongation of the inflammatory process, may lead to inflammatory diseases such as diabetes, asthma, atherosclerosis, cataracts, reperfusion injury, cancer, post-infectious syndromes such as in infectious meningitis, and rheumatic fever and rheumatic diseases such as systemic lupus erythematosus and rheumatoid arthritis. Additional inflammatory conditions that Ztnf11 can be used to treat include Inflammatory Bowel Disease, Ulcerative colitis, Crohn's Disease, and Irritable Bowel Syndrome.

The effect of Ztnf11, its analogs, agonists and/or antagonists, in a mouse model of LPS-induced mild endotoxemia can be used to measure the potential anti-inflammatory effects of therapeutic candidates during a robust inflammatory response. This model mimics acute endotoxemia/sepsis by challenging mice with a low, non-lethal dose of bacterial endotoxin (lipopolysaccharide, LPS). Serum is collected at various timepoints (1-8 hours) after intraperitoneal LPS injection and analyzed for altered expression of a wide variety of pro- and anti-inflammatory cytokines and acute phase proteins that mediate the inflammatory response. For example, six-month old Balb/c (Charles River Laboratories, Wilmington, Mass.) female mice are injected with 25 mg LPS (Sigma) in sterile PBS intraperitoneally (i.p.). Serum samples are collected at 0, 1, 4, 8, 16, 24, 48 and 72 hours from groups of 8 mice for each time point. Serum samples are assayed for inflammatory cytokine levels. Inflammatory mediators such as IL-1β, IL-6, TNFα, and IL-10 levels are measured using commercial ELISA kits purchased from Biosource International (Camarillo, Calif.). C57B1/6 mice (Charles River Laboratories; 5 mice/group) can then be treated i.p. with PBS, or varying concentrations of Ztnf 1l, its analogs, agonists and/or antagonists in PBS, 1 hour prior to LPS challenge. The mice are then challenged with 25 ug of LPS i.p. and bled at 1 hour and 4 hours after LPS injection. Serum is analyzed for the inflammatory mediator levels by ELISA.

Another model to measure immune response is the delayed type hypersensitivity (DTH) model which measures T cell responses to specific antigen. In this model, mice are immunized with a specific protein in adjuvant (e.g., chicken ovalbumin, OVA) and then later challenged with the same antigen (without adjuvant) in the ear. Increase in ear thickness (measured with calipers) after the challenge is a measure of specific immune response to the antigen. DTH is a form of cell-mediated immunity that occurs in three distinct phases 1) the cognitive phase, in which T cells recognize foreign protein antigens presented on the surface of antigen presenting cells (APCs), 2) the activation/sensitization phase, in which T cells secrete cytokines (especially interferon-gamma; IFN-g) and proliferate, and 3) the effector phase, which includes both inflammation (including infiltration of activated macrophages and neutrophils) and the ultimate resolution of the infection. This reaction is the primary defense mechanism against intracellular bacteria, and can be induced by soluble protein antigens or chemically reactive haptens. A classical DTH response occurs in individuals challenged with purified protein derivative (PPD) from Mycobacterium tuberculosis (TB), when those individuals injected have recovered from primary TB or have been vaccinated against TB. Induration, the hallmark of DTH, is detectable by about 18 hours after injection of antigen and is maximal by 24-48 hours. The lag in the onset of palpable induration is the reason for naming the response “delayed type.” In all species, DTH reactions are critically dependent on the presence of antigen-sensitized CD4+ (and, to a lesser extent, CD8+) T cells, which produce the principal initiating cytokine involved in DTH, IFN-g.

In order to test for anti-inflammatory effects of Ztnf11 in a DTH model, C57B1/6 mice are treated with: PBS and varying concentrations of Ztnf11, its analogs, agonists and/or antagonists. All of these treatments are given intraperitoneally two hours prior to the OVA re-challenge. The mice (8 per group) are first immunized in the back with 100 ug chicken ovalbumin (OVA) emulsified in Ribi in a total volume of 200 ul. Seven days later, the mice are re-challenged intradermally in the left ear with 10 ul PBS (control) or in the right ear with 10 ug OVA in PBS (no adjuvant) in a volume of 10 ul. Ear thickness of all mice is measured before injectiion in the ear (0 measurement). Ear thickness is measured 24 hours after challenge. The difference in ear thickness between the 0 measurement and the 24 hour measurement is recorded. Control mice in the PBS treatment group should develop a strong DTH reaction as shown by increase in the ear thickness at 24 hours post-challenge. A decrease in ear thickness as compared to the PBS control will indicate that Ztnf11, its analogs, agonists and/or antagonists, can reduce, limit, or ameliorate the inflammatory response.

The bioactive polypeptide or antibody conjugates described herein can be delivered intravenously, intraarterially or intraductally, or may be introduced locally at the intended site of action.

Moreover, inflammation is a protective response by an organism to fend off an invading agent. Inflammation is a cascading event that involves many cellular and humoral mediators. On one hand, suppression of inflammatory responses can leave a host immunocompromised; however, if left unchecked, inflammation can lead to serious complications including chronic inflammatory diseases (e.g., rheumatoid arthritis, multiple sclerosis, inflammatory bowel disease and the like), septic shock and multiple organ failure. Importantly, these diverse disease states share common inflammatory mediators. The collective diseases that are characterized by inflammation have a large impact on human morbidity and mortality. Therefore it is clear that anti-inflammatory antibodies and binding polypeptides, such as anti-ztnf11 antibodies and binding polypeptides described herein, could have crucial therapeutic potential for a vast number of human and animal diseases, from asthma and allergy to autoimmunity and septic shock. As such, use of anti-inflammatory anti ztnf11 antibodies and binding polypeptides described herein can be used therapeutically as ztnf11 antagonists, particularly in diseases such as arthritis, endotoxemia, inflammatory bowel disease, psoriasis, related disease and the like.

Arthritis, including osteoarthritis, rheumatoid arthritis, arthritic joints as a result of injury, and the like, are common inflammatory conditions which would benefit from the therapeutic use of anti-inflammatory antibodies and binding polypeptides, such as anti-ztnf11 antibodies and binding polypeptides of the present invention. For example, rheumatoid arthritis (RA) is a systemic disease that affects the entire body and is one of the most common forms of arthritis. It is characterized by the inflammation of the membrane lining the joint, which causes pain, stiffness, warmth, redness and swelling. Inflammatory cells release enzymes that may digest bone and cartilage. As a result of rheumatoid arthritis, the inflamed joint lining, the synovium, can invade and damage bone and cartilage leading to joint deterioration and severe pain amongst other physiologic effects. The involved joint can lose its shape and alignment, resulting in pain and loss of movement.

Rheumatoid arthritis (RA) is an immune-mediated disease particularly characterized by inflammation and subsequent tissue damage leading to severe disability and increased mortality. A variety of cytokines are produced locally in the rheumatoid joints. Numerous studies have demonstrated that IL-1 and TNF-alpha, two prototypic pro-inflammatory cytokines, play an important role in the mechanisms involved in synovial inflammation and in progressive joint destruction. Indeed, the administration of TNF-alpha and IL-1 inhibitors in patients with RA has led to a dramatic improvement of clinical and biological signs of inflammation and a reduction of radiological signs of bone erosion and cartilage destruction. However, despite these encouraging results, a significant percentage of patients do not respond to these agents, suggesting that other mediators are also involved in the pathophysiology of arthritis (Gabay, Expert. Opin. Biol. Ther. 2(2):135-149, 2002). One of those mediators could be ztnf11, and as such a molecule that binds or inhibits ztnf11, such as anti ztnf11 antibodies or binding partners, could serve as a valuable therapeutic to reduce inflammation in rheumatoid arthritis, and other arthritic diseases.

There are several animal models for rheumatoid arthritis known in the art. For example, in the collagen-induced arthritis (CIA) model, mice develop chronic inflammatory arthritis that closely resembles human rheumatoid arthritis. Since CIA shares similar immunological and pathological features with RA, this makes it an ideal model for screening potential human anti-inflammatory compounds. The CIA model is a well-known model in mice that depends on both an immune response, and an inflammatory response, in order to occur. The immune response comprises the interaction of B-cells and CD4+ T-cells in response to collagen, which is given as antigen, and leads to the production of anti-collagen antibodies. The inflammatory phase is the result of tissue responses from mediators of inflammation, as a consequence of some of these antibodies cross-reacting to the mouse's native collagen and activating the complement cascade. An advantage in using the CIA model is that the basic mechanisms of pathogenesis are known. The relevant T-cell and B-cell epitopes on type II collagen have been identified, and various immunological (e.g., delayed-type hypersensitivity and anti-collagen antibody) and inflammatory (e.g., cytokines, chemokines, and matrix-degrading enzymes) parameters relating to immune-mediated arthritis have been determined, and can thus be used to assess test compound efficacy in the CIA model (Wooley, Curr. Opin. Rheum. 3:407-20, 1999; Williams et al., Immunol. 89:9784-788, 1992; Myers et al., Life Sci. 61:1861-78, 1997; and Wang et al., Immunol. 92:8955-959, 1995).

The administration of soluble ztnf11 comprising polypeptides (including heterodimeric and multimeric receptors described herein), such as ztnf11-Fc4 or other ztnf11 soluble and fusion proteins to these CIA model mice is used to evaluate the use of ztnf11 to ameliorate symptoms and alter the course of disease. As a molecule that modulates immune and inflammatory response, ztnf11, may induce production of SAA, which is implicated in the pathogenesis of rheumatoid arthritis, ztnf11 antagonists may reduce SAA activity in vitro and in vivo, the systemic or local administration of ztnf11 antagonists such as anti-ztnf11 antibodies or binding partners, ztnf11 comprising polypeptides (including heterodimeric and multimeric receptors described herein), such as ztnf11-Fc4 or other ztnf11 soluble and fusion proteins can potentially suppress the inflammatory response in RA. Other potential therapeutics include ztnf11 polypeptides, soluble heterodimeric and multimeric receptor polypeptides, or anti ztnf11 antibodies or binding partners of the present invention, and the like.

Endotoxemia is a severe condition commonly resulting from infectious agents such as bacteria and other infectious disease agents, sepsis, toxic shock syndrome, or in immunocompromised patients subjected to opportunistic infections, and the like. Therapeutically useful of anti-inflammatory antibodies and binding polypeptides, such as anti-ztnf11 antibodies and binding polypeptides of the present invention, could aid in preventing and treating endotoxemia in humans and animals. Other potential therapeutics include ztnf11 polypeptides, soluble heterodimeric and multimeric receptor polypeptides, or anti ztnf11 antibodies or binding partners of the present invention, and the like, could serve as a valuable therapeutic to reduce inflammation and pathological effects in endotoxemia.

Lipopolysaccharide (LPS) induced endotoxemia engages many of the proinflammatory mediators that produce pathological effects in the infectious diseases and LPS induced endotoxemia in rodents is a widely used and acceptable model for studying the pharmacological effects of potential pro-inflammatory or immunomodulating agents. LPS, produced in gram-negative bacteria, is a major causative agent in the pathogenesis of septic shock (Glausner et al., Lancet 338:732, 1991). A shock-like state can indeed be induced experimentally by a single injection of LPS into animals. Molecules produced by cells responding to LPS can target pathogens directly or indirectly. Although these biological responses protect the host against invading pathogens, they may also cause harm. Thus, massive stimulation of innate immunity, occurring as a result of severe Gram-negative bacterial infection, leads to excess production of cytokines and other molecules, and the development of a fatal syndrome, septic shock syndrome, which is characterized by fever, hypotension, disseminated intravascular coagulation, and multiple organ failure (Dumitru et al. Cell 103:1071-1083, 2000).

These toxic effects of LPS are mostly related to macrophage activation leading to the release of multiple inflammatory mediators. Among these mediators, TNF appears to play a crucial role, as indicated by the prevention of LPS toxicity by the administration of neutralizing anti-TNF antibodies (Beutler et al., Science 229:869, 1985). It is well established that lug injection of E. coli LPS into a C57B1/6 mouse will result in significant increases in circulating IL-6, TNF-alpha, IL-1, and acute phase proteins (for example, SAA) approximately 2 hours post injection. The toxicity of LPS appears to be mediated by these cytokines as passive immunization against these mediators can result in decreased mortality (Beutler et al., Science 229:869, 1985). The potential immunointervention strategies for the prevention and/or treatment of septic shock include anti-TNF mAb, IL-1 receptor antagonist, LIF, IL-10, and G-CSF. Since LPS induces the production of pro-inflammatory factors possibly contributing to the pathology of endotoxemia, the neutralization of ztnf11 activity, SAA or other pro-inflammatory factors by antagonizing ztnf11 polypeptide can be used to reduce the symptoms of endotoxemia, such as seen in endotoxic shock. Other potential therapeutics include ztnf11 polypeptides, soluble heterodimeric and multimeric receptor polypeptides, or anti-ztnf11 antibodies or binding partners of the present invention, and the like.

In the United States approximately 500,000 people suffer from Inflammatory Bowel Disease (IBD) which can affect either colon and rectum (Ulcerative colitis) or both, small and large intestine (Crohn's Disease). The pathogenesis of these diseases is unclear, but they involve chronic inflammation of the affected tissues. Potential therapeutics include ztnf11 polypeptides, soluble heterodimeric and multimeric receptor polypeptides, or anti-ztnf11 antibodies or binding partners of the present invention, and the like., could serve as a valuable therapeutic to reduce inflammation and pathological effects in IBD and related diseases.

Ulcerative colitis (UC) is an inflammatory disease of the large intestine, commonly called the colon, characterized by inflammation and ulceration of the mucosa or innermost lining of the colon. This inflammation causes the colon to empty frequently, resulting in diarrhea. Symptoms include loosening of the stool and associated abdominal cramping, fever and weight loss. Although the exact cause of UC is unknown, recent research suggests that the body's natural defenses are operating against proteins in the body which the body thinks are foreign (an “autoimmune reaction”). Perhaps because they resemble bacterial proteins in the gut, these proteins may either instigate or stimulate the inflammatory process that begins to destroy the lining of the colon. As the lining of the colon is destroyed, ulcers form releasing mucus, pus and blood. The disease usually begins in the rectal area and may eventually extend through the entire large bowel. Repeated episodes of inflammation lead to thickening of the wall of the intestine and rectum with scar tissue. Death of colon tissue or sepsis may occur with severe disease. The symptoms of ulcerative colitis vary in severity and their onset may be gradual or sudden. Attacks may be provoked by many factors, including respiratory infections or stress.

Although there is currently no cure for UC available, treatments are focused on suppressing the abnormal inflammatory process in the colon lining.

Treatments including corticosteroids immunosuppressives (eg. azathioprine, mercaptopurine, and methotrexate) and aminosalicytates are available to treat the disease. However, the long-term use of immunosuppressives such as corticosteroids and azathioprine can result in serious side effects including thinning of bones, cataracts, infection, and liver and bone marrow effects. In the patients in whom current therapies are not successful, surgery is an option. The surgery involves the removal of the entire colon and the rectum.

There are several animal models that can partially mimic chronic ulcerative colitis. The most widely used model is the 2,4,6-trinitrobenesulfonic acid/ethanol (TNBS) induced colitis model, which induces chronic inflammation and ulceration in the colon. When TNBS is introduced into the colon of susceptible mice via intra-rectal instillation, it induces T-cell mediated immune response in the colonic mucosa, in this case leading to a massive mucosal inflammation characterized by the dense infiltration of T-cells and macrophages throughout the entire wall of the large bowel. Moreover, this histopathologic picture is accompanies by the clinical picture of progressive weight loss (wasting), bloody diarrhea, rectal prolapse, and large bowel wall thickening (Neurath et al. Intern. Rev. Immunol. 19:51-62, 2000).

Another colitis model uses dextran sulfate sodium (DSS), which induces an acute colitis manifested by bloody diarrhea, weight loss, shortening of the colon and mucosal ulceration with neutrophil infiltration. DSS-induced colitis is characterized histologically by infiltration of inflammatory cells into the lamina propria, with lymphoid hyperplasia, focal crypt damage, and epithelial ulceration.

These changes are thought to develop due to a toxic effect of DSS on the epithelium and by phagocytosis of lamina propria cells and production of TNF-alpha and IFN-gamma. Despite its common use, several issues regarding the mechanisms of DSS about the relevance to the human disease remain unresolved. DSS is regarded as a T cell-independent model because it is observed in T cell-deficient animals such as SCID mice.

The administration of anti-ztnf11 antibodies or binding partners, soluble ztnf11 comprising polypeptides (including heterodimeric and multimeric receptors), such as ztnf 1-Fc4 or other ztnf11 soluble and fusion proteins to these TNBS or DSS models can be used to evaluate the use of ztnf11 antagonists to ameliorate symptoms and alter the course of gastrointestinal disease. Ztnf11 may play a role in the inflammatory response in colitis, and the neutralization of ztnf11 activity by administrating ztnf11 antagonists is a potential therapeutic approach for IBD. Other potential therapeutics include ztnf11 polypeptides, soluble heterodimeric and multimeric receptor polypeptides, or anti-ztnf11 antibodies or binding partners of the present invention, and the like.

Psoriasis is a chronic skin condition that affects more than seven million Americans. Psoriasis occurs when new skin cells grow abnormally, resulting in inflamed, swollen, and scaly patches of skin where the old skin has not shed quickly enough. Plaque psoriasis, the most common form, is characterized by inflamed patches of skin (“lesions”) topped with silvery white scales. Psoriasis may be limited to a few plaques or involve moderate to extensive areas of skin, appearing most commonly on the scalp, knees, elbows and trunk. Although it is highly visible, psoriasis is not a contagious disease. The pathogenesis of the diseases involves chronic inflammation of the affected tissues. Ztnf11 polypeptides, soluble heterodimeric and multimeric receptor polypeptides, or anti-ztnf11 antibodies or binding partners of the present invention, and the like, could serve as a valuable therapeutic to reduce inflammation and pathological effects in psoriasis, other inflammatory skin diseases, skin and mucosal allergies, and related diseases.

Psoriasis is a T-cell mediated inflammatory disorder of the skin that can cause considerable discomfort. It is a disease for which there is no cure and affects people of all ages. Psoriasis affects approximately two percent of the populations of European and North America. Although individuals with mild psoriasis can often control their disease with topical agents, more than one million patients worldwide require ultraviolet or systemic immunosuppressive therapy. Unfortunately, the inconvenience and risks of ultraviolet radiation and the toxicities of many therapies limit their long-term use. Moreover, patients usually have recurrence of psoriasis, and in some cases rebound, shortly after stopping immunosuppressive therapy.

The effects of Ztnf11, its analogs, agonists and/or antagonists, on B cell proliferation can be measured in a B cell proliferation assay. For example, a vial containing 1×108 frozen, apheresed peripheral blood mononuclear cells (PBMCs) can be thawed in 37° C. water bath and resuspended in 25 ml B cell medium (Iscove's Modified Dulbecco's Medium, 10% Heat inactivated fetal bovine serum, 5% L-glutamine, 5% Pen/Strep) in a 50 ml tube (Falcon, VWR Seattle, Wash.). Cells are tested for viability using Trypan Blue (GIBCO BRL, Gaithersburg, Md.). Ten milliliters of Ficoll/Hypaque Plus (Pharmacia LKB Biotechnology Inc., Piscataway, N.J.) is layered under cell suspension and spun for 30 minutes at 1800 rpm and allowed to stop with the brake off. The interphase layer is then removed and transferred to a fresh 50 ml Falcon tube, brought up to a final volume of 40 ml with PBS and spun for 10 minutes at 1200 rpm with the brake on. The viability of the isolated B cells is tested using Trypan Blue. The B cells are resuspended at a final concentration of 1×106 cells/ml in B cell medium and plated at 180 μl/well in a 96 well U bottom plate (Falcon, VWR). One of the following stimulators are added to the cells to bring the final volume to 200 ml/well: Ztnf11 at 10 fold dilutions from 1 mg-1 ng/ml either alone, with 0.5% anti IgM (goat anti Human IgM-Agarose (μ chain specific) diluted in PBS, Sigma Chemical Co., St. Louis, Mo.); or with 0.5% anti IgM, and 10 ng/ml recombinant human IL4 (diluted in PBS and 0.1% BSA, Pharmingen, San Diego, Calif.). As a control the cells incubated with 0.1% bovine serum albumen (BSA) and PBS, 0.5% anti IgM or 0.5% anti IgM and 10 ng/ml IL4. The cells are then incubated at 37° C. in a humidified incubator for 72 hours. Sixteen hours prior to harvesting, 1 μCi 3H thymidine is added to all wells. The cells are harvested into a 96 well filter plate (UniFilter GF/C, Packard, Meriden, Conn.) are they harvested using a cell harvester (Packard) and collected according to manufacturer's instructions. The plates are dried at 55° C. for 20-30 minutes and the bottom of the wells are sealed with an opaque plate sealer. To each well is added 0.25 ml of scintillation fluid (Microscint-O, Packard) and the plate is read using a TopCount Microplate Scintillation Counter (Packard). In this assay, B cell stimulation over background controls shows B cell proliferation.

Additionally, assays to measure the effects of Ztnf11 on T cell proliferation, tumor proliferation, bone marrow progenitors, monocyte development are known to one of ordinary skill in the art.

The polypeptides, antagonists, agonists, nucleic acid and/or antibodies of the present invention may be used in treatment of disorders associated with immune function and inflammation. The molecules of the present invention may used to modulate or to treat or prevent development of pathological conditions in diverse tissue, including testis and lung. In particular, certain syndromes or diseases may be amenable to such diagnosis, treatment or prevention. In this sense, modulation of disease includes reduction, amelioration, limitation, and prevention of the inflammatory response or immune condition, disease, or disorder.

Additional methods using probes or primers derived, for example, from the nucleotide sequences disclosed herein can also be used to detect Ztnf11 expression in a patient sample, such as a blood, urine, semen, saliva, sweat, biopsy, tissue sample, or the like. For example, probes can be hybridized to tumor tissues and the hybridized complex detected by in situ hybridization. Ztnf11 sequences can also be detected by PCR amplification using cDNA generated by reverse translation of sample mRNA as a template (PCR Primer A Laboratory Manual, Dieffenbach and Dveksler, eds., Cold Spring Harbor Press, 1995). When compared with a normal control, both increases or decreases of Ztnf11 expression in a patient sample, relative to that of a control, can be monitored and used as an indicator or diagnostic for disease.

Moreover, the activity and effect of Ztnf11 on tumor progression and metastasis can be measured in vivo. Several syngeneic mouse models have been developed to study the influence of polypeptides, compounds or other treatments on tumor progression. In these models, tumor cells passaged in culture are implanted into mice of the same strain as the tumor donor. The cells will develop into tumors having similar characteristics in the recipient mice, and metastasis will also occur in some of the models. Tumor models include the Lewis lung carcinoma (ATCC No. CRL-1642) and B16 melanoma (ATCC No. CRL-6323), amongst others. These are both commonly used tumor lines, syngeneic to the C57BL6 mouse, that are readily cultured and manipulated in vitro. Tumors resulting from implantation of either of these cell lines are capable of metastasis to the lung in C57BL6 mice. The Lewis lung carcinoma model has recently been used in mice to identify an inhibitor of angiogenesis (O'Reilly M S, et al. Cell 79: 315-328,1994). C57BL6/J mice are treated with an experimental agent either through daily injection of recombinant protein, agonist or antagonist or a one time injection of recombinant adenovirus. Three days following this treatment, 10⁵ to 10⁶ cells are implanted under the dorsal skin. Alternatively, the cells themselves may be infected with recombinant adenovirus, such as one expressing Ztnf11, before implantation so that the protein is synthesized at the tumor site or intracellularly, rather than systemically. The mice normally develop visible tumors within 5 days. The tumors are allowed to grow for a period of up to 3 weeks, during which time they may reach a size of 1500-1800 mm³ in the control treated group. Tumor size and body weight are carefully monitored throughout the experiment. At the time of sacrifice, the tumor is removed and weighed along with the lungs and the liver. The lung weight has been shown to correlate well with metastatic tumor burden. As an additional measure, lung surface metastases are counted. The resected tumor, lungs and liver are prepared for histopathological examination, immunohistochemistry, and in situ hybridization, using methods known in the art and described herein. The influence of the expressed polypeptide in question, e.g., Ztnf11, on the ability of the tumor to recruit vasculature and undergo metastasis can thus be assessed. In addition, aside from using adenovirus, the implanted cells can be transiently transfected with Ztnf11. Moreover, purified Ztnf11 or Ztnf11-conditioned media can be directly injected in to this mouse model, and hence be used in this system. Use of stable Ztnf11 transfectants as well as use of induceable promoters to activate Ztnf11 expression in vivo are known in the art and can be used in this system to assess Ztnf11 induction of metastasis. For general reference see, O'Reilly M S, et al. Cell 79:315-328, 1994; and Rusciano D, et al. Murine Models of Liver Metastasis. Invasion Metastasis 14:349-361, 1995.

The invention also provides isolated and purified Ztnf11 polynucleotide probes. Such polynucleotide probes can be RNA or DNA. DNA can be either cDNA or genomic DNA. Polynucleotide probes are single or double-stranded DNA or RNA, generally synthetic oligonucleotides, but may be generated from cloned cDNA or genomic sequences and will generally comprise at least 16 nucleotides, more often from 17 nucleotides to 25 or more nucleotides, sometimes 40 to 60 nucleotides, and in some instances a substantial portion, domain or even the entire Ztnf11 gene or cDNA. The synthetic oligonucleotides of the present invention have at least 80% identity to a representative Ztnf12 DNA sequence (SEQ ID NO: 1) or its complements. Preferred regions from which to construct probes include the 5′ and/or 3′ coding sequences, receptor binding regions, extracellular, transmembrane and/or cytoplasmic domains, signal sequences and the like. Techniques for developing polynucleotide probes and hybridization techniques are known in the art, see for example, Ausubel et al., eds., Current Protocols in Molecular Biology, John Wiley and Sons, Inc., N.Y., 1991. For use as probes, the molecules can be labeled to provide a detectable signal, such as with an enzyme, biotin, a radionuclide, fluorophore, chemiluminescer, paramagnetic particle and the like, which are commercially available from many sources, such as Molecular Probes, Inc., (Eugene, Oreg.), and Amersham Corp., (Arlington Heights, Ill.), using techniques that are well known in the art.

Such probes can also be used in hybridizations to detect the presence or quantify the amount of Ztnf11 gene or mRNA transcript in a sample. Ztnf11 polynucleotide probes could be used to hybridize to DNA or RNA targets for diagnostic purposes, using such techniques such as fluorescent in situ hybridization (FISH) or immunohistochemistry.

Polynucleotide probes could be used to identify genes encoding Ztnf11-like proteins. For example, Ztnf11 polynucleotides can be used as primers and/or templates in PCR reactions to identify other novel members of the tumor necrosis factor family.

Such probes can also be used to screen libraries for related sequences encoding novel tumor necrosis factors. Such screening would be carried out under conditions of low stringency which would allow identification of sequences which are substantially homologous, but not requiring complete homology to the probe sequence. Such methods and conditions are well known in the art, see, for example, Sambrook et al., Molecular Cloning: A Laboratory Manual, Second Edition, Cold Spring Harbor, N.Y., 1989. Such low stringency conditions could include hybridization temperatures less than 42° C., formamide concentrations of less than 50% and moderate to low concentrations of salt. Libraries may be made of genomic DNA or cDNA.

Polynucleotide probes are also useful for Southern, Northern, or slot blots, colony and plaque hybridization and in situ hybridization. Mixtures of different Ztnf11 polynucleotide probes can be prepared which would increase sensitivity or the detection of low copy number targets, in screening systems.

Ztnf11 polypeptides may be used within diagnostic systems. Antibodies or other agents that specifically bind to Ztnf11 may be used to detect the presence of circulating ligand polypeptides. Such detection methods are well known in the art and include, for example, enzyme-linked immunosorbent assay (ELISA) and radioimmunoassay, immunohistochemically labeled antibodies can be used to detect Ztnf11 ligand in tissue samples. Ztnf11 levels can also be monitored by such methods as RT-PCR, where Ztnf11 mRNA can be detected and quantified. Such methods could be used as diagnostic tools to monitor and quantify receptor or ligand polypeptide levels. The information derived from such detection methods would provide insight into the significance of Ztnf11 polypeptides in various diseases, and as a would serve as diagnostic methods for diseases for which altered levels of Ztnf11 are significant. Altered levels of Ztnf11 ligand polypeptides may be indicative of pathological conditions including cancer, autoimmune disorders, inflammation and immunodeficiencies.

The Ztnf11 polynucleotides and/or polypeptides disclosed herein can be useful as therapeutics, wherein Ztnf11 agonists and/or antagonists could modulate one or more biological processes in cells, tissues and/or biological fluids. Many members of the TNF family are expressed on lymphoid cells and mediate interactions between different immune cells. The homology of Ztnf11 with TNF suggests that Ztnf11 plays a role in regulation of the immune response, including the activation and regulation of lymphocytes. Ztnf11 polypeptides and Ztnf11 agonists would be useful as therapies for treating immunodeficiencies. The Ztnf11 polypeptides, Ztnf11 agonists and antagonists could be employed in therapeutic protocols for treatment of such autoimmune diseases as insulin dependent diabetes mellitus (IDDM), Crohn's Disease, muscular sclerosis (MS), myasthenia gravis (MG) and systemic lupus erythematosus.

Ztnf11 polypeptides and Ztnf11 agonists can be used to regulate anti-viral response, in treatments to combat infection and to provide relief from allergy symptoms. Ztnf11 polypeptides and Ztnf11 agonists can also be used to inhibit cancerous cell growth by acting as a mediator of cell apoptosis. Ztnf11 polypeptides and Ztnf11 agonists are also contemplated for use in regulation of certain carcinomas, such as lung carcinomas, small-cell cancers, squamous-cell carcinomas, large-cell carcinomas and adenocarcinomas.

Ztnf11 polynucleotides and polypeptides can be used as standards to calibrate in vitro cytokine assay systems or as standards within such assay systems. In addition, antibodies to Ztnf11 polypeptides could be used in assays for neutralization of bioactivity, in ELISA and ELISPOT assays, in Western blot analysis and for immunohistochemical applications. Various other cytokine proteins, antibodies and DNA are available from numerous commercial sources, such as R & D Systems, Minneapolis, Minn., for use in such methodologies.

The invention also provides antagonists, which either bind to Ztnf11 polypeptides or, alternatively, to a receptor to which Ztnf11 polypeptides bind, thereby inhibiting or eliminating the function of Ztnf11. Such Ztnf11 antagonists would include antibodies; oligonucleotides which bind either to the Ztnf11 polypeptide or to its receptor; natural or synthetic analogs of Ztnf11 polypeptides which retain the ability to bind the receptor but do not result in either ligand or receptor signaling. Such analogs could be peptides or peptide-like compounds. Natural or synthetic small molecules, which bind to receptors of Ztnf11 polypeptides and prevent signaling, are also contemplated as antagonists. As such, Ztnf11 antagonists would be useful as therapeutics for treating certain disorders where blocking signal from either a Ztnf11 ligand or receptor would be beneficial.

Antagonists would have additional therapeutic value for treating chronic inflammatory diseases, for example, to lessen joint pain, swelling, anemia and other associated symptoms. Antagonists may also be useful in preventing bone resorption. They could also find use in treatments for rheumatoid arthritis and systemic lupus erythematosius. Antagonists would also find use in treating septic shock.

Ztnf11 polypeptides and Ztnf11 polypeptide antagonists can be employed in the study of effector functions of T lymphocytes, in particular T lymphocyte activation and differentiation. Also in T helper functions in mediating humoral or cellular immunity. Ztnf11 polypeptides and Ztnf11 polypeptide antagonists are also contemplated as useful research reagents for characterizing ligand-receptor interactions.

The invention also provides nucleic acid-based therapeutic treatment. If a mammal has a mutated or lacks a Ztnf11 gene, the Ztnf11 gene can be introduced into the cells of the mammal. In one embodiment, a gene encoding a Ztnf11 polypeptide is introduced in vivo in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes virus 1 (HSV1) vector (Kaplitt et al., Molec. Cell. Neurosci. 2:320-30, 1991), an attenuated adenovirus vector, such as the vector described by Stratford-Perricaudet et al. (J. Clin. Invest. 90:626-30, 1992), and a defective adeno-associated virus vector (Samulski et al., J. Virol. 61:3096-101, 1987; Samulski et al., J. Virol. 63:3822-8, 1989).

In another embodiment, the gene can be introduced in a retroviral vector, e.g., as described in Anderson et al., U.S. Pat. No. 5,399,346; Mann et al., Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; Dougherty et al., WIPO Publication WO 95/07358; and Kuo et al., Blood 82:845-52, 1993.

Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-17, 1987; and Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. It is clear that directing transfection to particular cells represents one area of benefit. It is clear that directing transfection to particular cell types would be particularly advantageous in a tissue with cellular heterogeneity, such as the pancreas, liver, kidney, and brain. Lipids may be chemically coupled to other molecules for the purpose of targeting. Targeted peptides, e.g., hormones or neurotransmitters, and proteins such as antibodies, or non-peptide molecules could be coupled to liposomes chemically.

It is possible to remove the cells from the body and introduce the vector as a naked DNA plasmid and then re-implant the transformed cells into the body. Naked DNA vector for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter (see, for example, Wu et al., J. Biol. Chem. 267:963-7, 1992; Wu et al., J. Biol. Chem. 263:14621-24, 1988).

The Ztnf11 polypeptides are also contemplated for pharmaceutical use. Pharmaceutically effective amounts of Ztnf11 polypeptides, agonists or Ztnf11 antagonists of the present invention can be formulated with pharmaceutically acceptable carriers for parenteral, oral, nasal, rectal, topical, intramuscular, transdermal administration or the like, according to conventional methods. Formulations may further include one or more diluents, fillers, emulsifiers, preservatives, buffers, excipients, and the like, and may be provided in such forms as liquids, powders, emulsions, suppositories, liposomes, transdermal patches and tablets, for example. Slow or extended-release delivery systems, including any of a number of biopolymers (biological-based systems), systems employing liposomes, and polymeric delivery systems, can also be utilized with the compositions described herein to provide a continuous or long-term source of the Ztnf11 polypeptide or antagonist. Such slow release systems are applicable to formulations, for example, for oral, topical and parenteral use. The term “pharmaceutically acceptable carrier” refers to a carrier medium which does not interfere with the effectiveness of the biological activity of the active ingredients and which is not toxic to the host or patient. One skilled in the art may formulate the compounds of the present invention in an appropriate manner, and in accordance with accepted practices, such as those disclosed in Remington's Pharmaceutical Sciences, Gennaro (ed.), Mack Publishing Co., Easton, Pa. 1990.

As used herein a “pharmaceutically effective amount” of a Ztnf11 polypeptide, agonist or antagonist is an amount sufficient to induce a desired biological result. The result can be alleviation of the signs, symptoms, or causes of a disease, or any other desired alteration of a biological system. For example, an effective amount of a Ztnf11 polypeptide or antagonist is that which provides either subjective relief of symptoms or an objectively identifiable improvement as noted by the clinician or other qualified observer. It may also be an amount which results in reduction of serum Ca⁺⁺ levels or an inhibition of osteoclast size and number in response to treatment for bone resorption. Other such examples include reduction in acetylcholine antibody levels, a decrease in muscle weakness during treatment for myasthenia gravis; or other beneficial effects. Effective amounts of Ztnf11 for use in treating multiple sclerosis (MS) would result in decrease in muscle weakness, and/or a reduction in frequency of MS exacerbation. In EAE mouse model measurements, EAE grades, of clinical signs of disease, such as limp tail or degree of paralysis are made. For rheumatoid arthritis, such indicators include a reduction in inflammation and relief of pain or stiffness, in animal models indications would be derived from macroscopic inspection of joints and change in swelling of hind paws. Effective amounts of the Ztnf11 polypeptides can vary widely depending on the disease or symptom to be treated. The polypeptides, polynucleotides, and antibodies of the present invention, as well as fragments thereof will be useful in treating diseases including, hematopoeisis, inflammation, cellular deficiencies, abnormal cellular proliferation, apoptosis, and cancers. Additionally, the polypeptides, polynucleotides, and antibodies of the present invention, as well as fragments thereof will be useful in treating immune and/or inflammation disorders, such as diabetes, asthma, atherosclerosis, cataracts, reperfusion injury, post-infectious syndromes such as in infectious meningitis, and rheumatic fever and rheumatic diseases such as systemic lupus erythematosus and rheumatoid arthritis, Inflammatory Bowel Disease, Ulcerative colitis, Crohn's Disease, and Irritable Bowel Syndrome.

The amount of the polypeptide to be administered and its concentration in the formulations, depends upon the vehicle selected, route of administration, the potency of the particular polypeptide, the clinical condition of the patient, the side effects and the stability of the compound in the formulation. Thus, the clinician will employ the appropriate preparation containing the appropriate concentration in the formulation, as well as the amount of formulation administered, depending upon clinical experience with the patient in question or with similar patients. Such amounts will depend, in part, on the particular condition to be treated, age, weight, and general health of the patient, and other factors evident to those skilled in the art. Typically a dose will be in the range of 0.1-100 mg/kg of subject. Doses for specific compounds may be determined from in vitro or ex vivo studies in combination with studies on experimental animals. Concentrations of compounds found to be effective in vitro or ex vivo provide guidance for animal studies, wherein doses are calculated to provide similar concentrations at the site of action. Doses determined to be effective in experimental animals are generally predictive of doses in humans within one order of magnitude.

The dosages of the present compounds used to practice the invention include dosages effective to result in the desired effects. Estimation of appropriate dosages effective for the individual patient is well within the skill of the ordinary prescribing physician or other appropriate health care practitioner. As a guide, the clinician can use conventionally available advice from a source such as the Physician's Desk Reference, 48^{th} Edition, Medical Economics Data Production Co., Montvale, N.J. 07645-1742 (1994).

Preferably the compositions are presented for administration in unit dosage forms. The term “unit dosage form” refers to physically discrete units suitable as unitary dosed for human subjects and animals, each unit containing a predetermined quantity of active material calculated to produce a desired pharmaceutical effect in association with the required pharmaceutical diluent, carrier or vehicle. Examples of unit dosage forms include vials, ampules, tablets, caplets, pills, powders, granules, eyedrops, oral or ocular solutions or suspensions, ocular ointments, and oil-in-water emulsions. Means of preparation, formulation and administration are known to those of skill, see generally Remington's Pharmaceutical Science 15^{th} ed., Mack Publishing Co., Easton, Pa. (1990).

The invention is further illustrated by the following non-limiting examples.

### EXAMPLES

### Example 1

### Construction of Soluble Ztnf11 Expression Vectors

An expression vector is prepared to express the soluble ztnf11 polypeptide fused to a C-terminal Glu-Glu tag.

A PCR generated Ztnf11 DNA fragment is created using appropriate oligonucleotides as PCR primers to add suitable restriction sites at 5′ and 3′ ends of the soluble Ztnf11 DNA. A plasmid containing the Ztnf11 cDNA (SEQ ID NO:1) is used as a template for PCR amplification. The reaction is purified by chloroform/phenol extraction and isopropanol precipitation, and digested with the selected restriction endonucleases (Boehringer Mannheim, Indianapolis, Ind.). A band of the appropriate length is visualized by 1% agarose gel electrophoresis, excised, and the DNA is purified using a QiaexII™ purification kit (Qiagen, Valencia, Calif.) according to the manufacturer's instruction.

About 30 ng of the restriction digested Ztnf11 insert and about 10 ng of an appropriate digested expression vector is ligated at room temperature for 2 hours.

One microliter of ligation reaction is electroporated into DH10B competent cells (Gibco BRL, Rockville, Md.) according to manufacturer's direction and plated onto LB plates containing 50 mg/ml ampicillin, and incubated overnight. Colonies are screened by restriction analysis of DNA, which is prepared from 2 ml liquid cultures of individual colonies. The insert sequence of positive clones is verified by sequence analysis. Thus, the excised Ztnf11 DNA is subcloned into the appropriate expression vector. A large-scale plasmid preparation is done using a Qiagen® Mega prep kit (Qiagen) according to manufacturer's instruction.

The same process is used to prepare the Ztnf11 with a C-terminal Fc4 tag, creating the Ztnf11/Fc4. To prepare Ztnf11/Fc4, the expression vector has a Fc4 tag in place of the Glu-Glu tag. Fc4 is the Fc region derived from human IgG, which contains a mutation so that it no longer binds the Fc receptor. Although Fc4 is utilized in the present example, one of ordinary skill recognizes that other Fc constructs (i.e., those derived from other Ig molecules) can be used to prepare a soluble Ztnf11 utilizing this same protocol.

### Example 2

### Transfection and Expression of Ztnf11 Soluble Polypeptides

The day before the transfection, BHK 570 cells (ATCC No. CRL-10314; ATCC, Manasas, Va.) are plated in a 10-cm plate with 50% confluence in normal BHK DMEM (Gibco/BRL High Glucose) media. The day of the transfection, the cells are washed once with Serum Free (SF) DMEM, followed by transfection with the Ztnf11/Fc4 or Ztnf11/CEE expression plasmids. Sixteen micrograms of each DNA construct are separately diluted into a total final volume of 640 μl SF DMEM. A diluted LipofectAMINE™ mixture (35 μl LipofectAMINE™ in 605 μl SF meida) is added to the DNA mix, and incubated for 30 minutes at room temperature. Five milliliters of SF media is added to the DNA/LipofectAMINE™ mixture, which is then added to BHK cells. The cells are incubated at 37° C./5% CO2 for 5 hours, after which 6.4 ml of BHK media with 10% FBS is added. The cells are incubated overnight at 37° C./5% CO2.

Approximately 24 hours post-transfection, the BHK cells are split into selection media with 1 uM methotrexate (MTX). The cells are repeatedly split in this manner until stable Ztnf11/CEE and Ztnf11/Fc4 cell lines are identified. To detect the expression level of the Ztnf11 soluble fusion proteins, the BHK cells are washed with PBS and incubated in SF media for 72 hours. The SF condition media is collected and 20 μl of the sample is run on 10% SDS-PAGE gel under reduced conditions. The protein bands are transferred to nitrocellulose filter by Western blot, and the fusion proteins are detected using either goat-anti-human IgG/HRP conjugates for the Ztnf11/Fc4 fusion or mouse-anti-Glu-Glu tag/HRP conjugates for the Ztnf11/CEE fusion. Expression vectors containing a different soluble fused to the Fc4 or the CEE tags are used as controls.

Transfected BHK cells are transferred into T-162 flasks. Once the BHK cells reached about 80% confluence, they are washed with PBS and incubated in 100 ml SF media for 72 hours, and then the condition media is collected for protein purification.

### Example 3

### Purification and Analysis of Ztnf11/CEE

Recombinant carboxyl terminal Glu-Glu tagged Ztnf11 is produced from transfected BHK cells as described in Example 2 above. About six liters of conditioned media are harvested from 60 dishes after roughly 72 hours incubation. A portion of the media is sterile filtered using filtration units from different manufactures. The Nalgene 0.2 μm and 0.45 μm filters, and Millipore Express 0.22 μm filter are compared and the one providing the best recovery of the protein and flow rate is used. The level of protein expression reaches the optimal concentration after about 72 hours in new media.

Protein is purified from the filtered media by a combination of Anti-Glu-Glu (Anti-EE) peptide antibody affinity chromatography and S-100 gel exclusion chromatography. Culture medium is directly loaded onto a 20×185 mm (58-ml bed volume) anti-EE antibody affinity column at a flow of about 4 ml/minute. Following column washing with ten column volumes of PBS, bound protein is eluted with two column volumes of 0.4 mg/ml EYMPTD peptide (Princeton Biomolectiles, N.J.). Fractions of 5 ml are collected. Samples from the anti-EE antibody affinity column are analyzed by SDS-PAGE with silver staining and western blotting for the presence of Ztnf11/CEE. Fractions containing the Ztnf11/CEE protein are pooled and concentrated to 4 mls using Biomax-5 concentrator (Millipore), and loaded onto a 16×1000 mm Sephacryl S-100 HR gel filtration column (Amersham Pharmacia Biotech). The fractions containing purified Ztnf11/CEE are pooled, filtered through 0.2 μm filter, aliquoted into 100 μl each, and frozen at −80° C. The concentration of the final purified protein is determined by BCA assay (Pierce) and HPLC-amino acid analysis.

Recombinant Ztnf11/CEE is analyzed by SDS-PAGE (Nupage 4-12%), Novex) with either coomassie and silver staining method (Fast Silver, Geno Tech), and Western blotting using monoclonal anti-EE antibody. Either the conditioned media or purified protein is electrophoresed using a Novex's Xcell II mini-cell (San Diego, Calif.) and transferred to nitrocellulose (0.2 μm; Bio-Rad Laboratories, Hercules, Calif.) at room temperature using Novex's Xcell II blot module with stirring according to directions provided in the instrument manual. The transfer is run at 500 mA for one hour in a buffer containing 25 mM Tris base, 200 mM glycine, and 20% methanol. The filters are then blocked with 10% non-fat dry milk in PBS for 10 minutes at room temperature. The nitrocellulose is quickly rinsed, then primary antibody is added in PBS containing 2.5% non-fat dry milk. The blots are incubated for two hours at room temperature or overnight at 4° C. with gentle shaking. Following the incubation, blots are washed three times for 10 minutes each in PBS. Secondary antibody (goat anti-mouse IgG conjugated to horseradish peroxidase; obtained from Rockland Inc., Gilbertsville, Pa.) diluted 1:2000 in PBS containing 2.5% non-fat dry milk is added, and the blots are incubated for two hours at room temperature with gentle shaking. The blots are then washed three times, 10 minutes each, in PBS, then quickly rinsed in H₂O. The blots are developed using commercially available chemiluminescent substrate reagents (SuperSignalÒ ULTRA reagents 1 and 2 mixed 1:1; reagents obtained from Pierce Chemical Co.), and the signal is captured using Lumi-Imager's Lumi Analyst 3.0 software (Boehringer Mannheim GmbH, Germany) for exposure times ranging from 10 second to 5 minutes or as necessary.

### Example 4

### Purification and Analysis of Ztnf11/Fc4

Recombinant carboxyl terminal Fc4 tagged Ztnf11 is produced from transfected BHK cells as described in Example 2 above. Approximately five-liters of conditioned media are harvested from 60 dishes after about 72 hours of incubation. A portion of the media is sterile filtered using filtration units from different manufactures. The Nalgene 0.2 μm and 0.45 μm filters, Millipore Express 0.22 μm filter, and Durapore 0.45 μm filter are compared and the one providing the best yield and flow rate is used. The level of protein expression reaches the optimal concentration after about 72 hours in the new media.

Protein is purified from the filtered media by a combination of Poros 50 protein A affinity chromatography (PerSeptive Biosystems, 1-5559-01, Framingham, Mass.) and S-200 gel exclusion chromatography column (Amersham Pharmacia Biotech). Culture medium is directly loaded onto a 10×80 mm (6.2-ml bed volume) protein A affinity column at a flow of about 4 ml/minute. Following column washing for ten column volumes of PBS, bound protein is eluted by five column volumes of 0.1 M glycine, pH 3.0 at 10 ml/minute). Fractions of 1.5 ml each are collected into tubes containing 38 μl of 2.0 M Tris, pH 8.8, in order to neutralize the eluted proteins. Samples from the affinity column are analyzed by SDS-PAGE with Coomassie staining and Western blotting for the presence of Ztnf11/Fc4 using human IgG-HRP. Ztnfr11/Fc4-containing fractions are pooled and concentrated to 4 mls using Biomax-30 concentrator (Millipore), and loaded onto a 16×1000 mm Sephacryl S-200 HR gel filtration. The fractions containing purified Ztnf11/Fc4 are pooled, filtered through 0.2 μm filter, aliquoted into 100, 200 and 500 μl each, and frozen at −80° C. The concentration of the final purified protein is determined by BCA assay (Pierce) and HPLC-amino acid analysis.

Recombinant Ztnf11/Fc4 is analyzed by SDS-PAGE (Nupage 4-12%, Novex) with coomassie staining method and Western blotting using human IgG-HRP. Either the conditioned media or purified protein is electrophoresed using a Novex's Xcell II mini-cell (San Diego, Calif.) and transferred to nitrocellulose (0.2 μm; Bio-Rad Laboratories, Hercules, Calif.) at room temperature using Novex's Xcell II blot module with stirring according to directions provided in the instrument manual. The transfer is run at 500 mA for one hour in a buffer containing 25 mM Tris base, 200 mM glycine, and 20% methanol. The filters are then blocked with 10% non-fat dry milk in PBS for 10 minutes at room temperature. The nitrocellulose is quickly rinsed, then the human Ig-HRP antibody (1:2000) is added in PBS containing 2.5% non-fat dry milk. The blots are incubated for two hours at room temperature, or overnight at 4° C., with gentle shaking. Following the incubation, the blots are washed three times for 10 minutes each in PBS, then quickly rinsed in H2O. The blots are developed using commercially available chemiluminescent substrate reagents (SuperSignalÒ ULTRA reagents 1 and 2 mixed 1:1; reagents obtained from Pierce Chemical Co.), and the signal is captured using Lumi-Imager's Lumi Analyst 3.0 software (Boehringer Mannheim GmbH, Germany) for exposure times ranging from 10 second to 5 minutes or as necessary.

### Example 5

### Identification of Cells Expressing Ztnf11 Using In Situ Hybridization

Specific human tissues are isolated and screened for Ztnf11 expression by in situ hybridization. Various human tissues prepared, sectioned and subjected to in situ hybridization includes normal stomach, normal uterus, neuroblastomas and melanoma, among other cancers. The tissues are fixed in 10% buffered formalin and blocked in paraffin using standard techniques. Tissues are sectioned at 4 to 8 microns. Tissues are prepared using a standard protocol (“Development of non-isotopic in situ hybridization” at http://dir.niehs.nih.gov/dirlep/ish.html). Briefly, tissue sections are deparaffinized with HistoClear (National Diagnostics, Atlanta, Ga.) and then dehydrated with ethanol. Next they are digested with Proteinase K (50 mg/ml) (Boehringer Diagnostics, Indianapolis, Ind.) at 37° C. for 2 to 20 minutes. This step is followed by acetylation and re-hydration of the tissues.

Two in situ probes generated by PCR are designed against the human Ztnf11 sequence. Two sets of oligos are designed to generate probes for separate regions of the Ztnf11 cDNA. The antisense oligo from each set also contains the working sequence for the T7 RNA polymerase promoter to allow for easy transcription of antisense RNA probes from these PCR products. The probes are made by PCR amplification. Probes are subsequently labeled with digoxigenin (Boehringer) or biotin (Boehringer) using an In Vitro transcription System (Promega, Madison, Wis.) as per manufacturer's instruction.

In situ hybridization is performed with a digoxigenin- or biotin-labeled Ztnf11 probe. The probe is added to the slides at a concentration of 1 to 5 pmol/ml for 12 to 16 hours at 60° C. Slides are subsequently washed in 2×SSC and 0.1×SSC at 55° C. The signals are amplified using tyramide signal amplification (TSA) (TSA, in situ indirect kit; NEN) and visualized with Vector Red substrate kit (Vector Lab) as per manufacturer's instructions. The slides are then counter-stained with hematoxylin (Vector Laboratories, Burlingame, Calif.).

### Example 6

### Human Ztnf11 Polyclonal Antibodies

Polyclonal antibodies are prepared by immunizing 2 female New Zealand white rabbits with the purified recombinant protein Ztnf11-CEE protein expressed in BHK from Example 2. The rabbits are each given an initial intraperitoneal (ip) injection of 200 μg of purified protein in Complete Freund's Adjuvant followed by booster ip injections of 100 μg peptide in Incomplete Freund's Adjuvant every three weeks. Seven to ten days after the administration of the second booster injection (3 total injections), the animals are bled and the serum is collected. The animals are then boosted and bled every three weeks.

The Ztnf11-specific polyclonal antibodies are affinity purified from the rabbit serum using a CNBr-SEPHAROSE 4B protein column (Pharmacia LKB) that is prepared using 10 mg of purified recombinant Ztnf11-Fc protein per gram of CNBr-SEPHAROSE, followed by 20× dialysis in PBS overnight. Ztnfr11-specific antibodies are characterized by ELISA using 1 μg/ml of the specific purified recombinant Ztnf11-CEE-BHK protein as antibody target.

### Example 7

### Tissue Distribution in cDNA Panels Using PCR

Nine panels of 1^{st} strand cDNAs from human tissues or cell lines were screened for ztnf11 expression using PCR. The panels were made in-house and contained 378 1^{st} strand cDNA samples from various human tissues (normal, cancer, and diseased) and resting or stimulated cell lines shown in Table 5, below. The 1^{st} strand cDNA for the 1^{st} strand cDNAs plates were generated from in-house RNA preps, Clontech (Palo Alto, Calif.) RNA, or Invitrogen (Carlsbad, Calif.) RNA. To assure quality of the panel samples, a PCR was run using clathrin primers zc21, 195 (SEQ ID NO: 18) and zc21, 196 (SEQ ID NO: 19) and an extension time of 1 minute at 68° C. The panels were set up in a 96-well format that included 100 ng human genomic DNA (Clontech, Palo Alto, Calif.) as a positive control sample. Each well contained 1^{st} strand cDNA synthesized from 100 ng of total RNA. The PCR reactions were set up using 0.5 μl of 20 uM each of oligos ZC47124 (SEQ ID NO:20) and ZC47127 (SEQ ID NO:21), 2.5 ul 10× buffer and 0.5 ul Advantage 2 cDNA polymerase mix (BD Biosciences Clontech, Palo Alto, Calif.), 1 ul 2.5 mM dNTP mix (Applied Biosystems, Foster City, Calif.), 10% DMSO (Sigma, St. Louis, Mo.) and 1× Rediload dye (Invitrogen, Carlsbad, Calif.) in a final volume of 27.5 ul. The amplification was carried out as follows: 1 cycle at 94° C. for 2 minutes, 35 cycles of 94° C. for 30 seconds, 64° C. for 30 seconds and 72° C. for 45 seconds, followed by 1 cycle at 72° C. for 5 minutes. About 10 μl of the PCR reaction product was subjected to standard agarose gel electrophoresis using a 4% agarose gel. There is a long (×1) and short (×2) form of ztnf11. The oligos can pick up both forms. They differ by 564 bp. The long form is 669 bp and the short form is 105 bp. See Table 5 below for expression profile for both forms and tissues screened. The genomic band is 861 bp in size.

DNA fragments were excised from thyroid, lymphoma, lung, 3AsubE FS, A-172 cytoplasmic, colon, stomach, endometruim, and testis; then purified using a Gel Extraction Kit (Qiagen, Chatsworth, Calif.) according to manufacturer's instructions. Fragments were confirmed by sequencing to show that they were indeed ztnf11×1, ztnf11×2.

The PCR results indicate that ztnf11×2 mRNA expression is extremely rare. No cell line produces ztnf11×2 mRNA in this set of assays, and only a handful of tissues express this splice variant, the most consistent expression being in 3 of 5 normal brain tissues. Ztnf11×2 mRNA is also seen in one tissue each of multiple tissue samples from parotid gland, trachea, endometrium, testis, and prostate.

In constrast, the PCR results for ztnf11×1, the longer form, indicate that ztnf11×1 mRNA is not ubiquitous, but more broadly expressed than ztnf11×2, still being somewhat restricted in it's expression. In cell lines, ztnf11×1 is most consistently expressed in immune-related cell lines, as seen by the results from plate #117, below in Table 5. Within that category, the monocyte cell lines U-937 and THP-1 are the most consistent expressors of ztnf11×1. Interestingly, CD3+ T-cells and NK cells are consistently expressing ztnf11×1 when peripheral blood fraction PCR results are examined. Within tissues, ztnf11×1 expression appears to be common in digestive system tissues, such as small intestine, stomach and to a lesser degree, colon. This pattern is reflected in ztnf11×1 mRNA expression in cell lines derived from digestive system tissues, such as CaCO2, FHS74.Int, and Int407. Expression of ztnf11×1 is also observed regularly in lung tissues and lung-derived cell lines such as MRC-5, Sk-Lu-1, and WI38. In parallel to the northern blot results, ztnf11×1 scores positive in most testis and prostate tissues and cell lines. Scattered ztnf11×1 expression is also observed in brain tissues and cell lines derived from brain such as U373MG, A-172, and Sk-N-SH. Although large numbers of skin tissues were not available to survey for ztnf11 expression, many of the skin-derived cell lines were positive for ztnf11×1 mRNA, including Malme3m, SkMEL-2, HT144, G361, and Hs294T. Finally, ztnf11×1 expression is observed occasionally in endocrine tissues such as pancreas, ovary, and thyroid.

TABLE 5

| | | | | | Ztnf11x1 | Ztnf11x2 |
|---|---|---|---|---|---|---|
| | | | | | Long | Short |
| Plate # | Row | Col. | Tissue | Health | Form | Form |
| 109 | A | 1 | Raji | cancer | No | No |
| 109 | B | 1 | Bjab | cancer | No | No |
| 109 | C | 1 | CCRF-CEM | cancer | No | No |
| 109 | D | 1 | Cess | cancer | No | No |
| 109 | E | 1 | Daudi | unknown | No | No |
| 109 | F | 1 | HEL 92.1.7 | cancer | No | No |
| 109 | G | 1 | HL-60 | cancer | No | No |
| 109 | H | 1 | HL-60 + PMA | cancer | Yes | No |
| 109 | A | 2 | HL-60 + PMA 24 hr | cancer | No | No |
| 109 | B | 2 | HS Sultan | cancer | No | No |
| 109 | C | 2 | TE - negative control | | No | No |
| 109 | D | 2 | HUT 78 cytoplasmic | cancer | No | No |
| 109 | E | 2 | IM-9 | cancer | No | No |
| 109 | F | 2 | Jurkat | cancer | No | No |
| 109 | G | 2 | K562 | cancer | Yes | No |
| 109 | H | 2 | Molt4 | cancer | No | No |
| 109 | A | 3 | Ramos | cancer | No | No |
| 109 | B | 3 | RL | cancer | No | No |
| 109 | C | 3 | opm-2 | cancer | No | No |
| 109 | D | 3 | RPMI 1788 | normal | No | No |
| 109 | E | 3 | ARH-77 | cancer | No | No |
| 109 | F | 3 | RPMI 7961 | unknown | No | No |
| 109 | G | 3 | RPMI 8226 | cancer | No | No |
| 109 | H | 3 | Sk-N-SH | cancer | Yes | No |
| 109 | A | 4 | TF-1 | cancer | Yes | No |
| 109 | B | 4 | THP-1 | cancer | Yes | No |
| 109 | C | 4 | THP-1 + LPS + gIFN | cancer | Yes | No |
| 109 | D | 4 | TrBMEC | normal | Yes | No |
| 109 | E | 4 | U-937 | cancer | Yes | No |
| 109 | F | 4 | U-937b + PMA + iono. | cancer | Yes | No |
| 109 | G | 4 | Granta-519 | cancer | No | No |
| 109 | H | 4 | 3AsubE FS | normal | Yes | No |
| 109 | A | 5 | Mg-63 | cancer | Maybe | No |
| 109 | B | 5 | L-363 | cancer | No | No |
| 109 | C | 5 | U-2 OS | cancer | Yes | No |
| 109 | D | 5 | 5637 | cancer | Maybe | No |
| 109 | E | 5 | Caco-2 | cancer | Yes | No |
| 109 | F | 5 | Caco-2, diff. | cancer | Yes | No |
| 109 | G | 5 | DLD-1 | cancer | No | No |
| 109 | H | 5 | WM-115 cytoplasmic | unknown | Yes | No |
| 109 | A | 6 | FHS74.Int | unknown | Yes | No |
| 109 | B | 6 | HCT-116 | cancer | No | No |
| 109 | C | 6 | HCT-15 | cancer | No | No |
| 109 | D | 6 | HT-29 | cancer | No | No |
| 109 | E | 6 | INT407 | normal | Yes | No |
| 109 | F | 6 | CASMC | normal | No | No |
| 109 | G | 6 | ARPE-19 | normal | No | No |
| 109 | H | 6 | WERI-Rb-1 | cancer | No | No |
| 109 | A | 7 | Y-79 | cancer | Yes | No |
| 109 | B | 7 | HBL-100 | no info | Yes | No |
| 109 | C | 7 | MCF7 | cancer | Yes | No |
| 109 | D | 7 | DOHH-2 | unknown | No | No |
| 109 | E | 7 | HepG2 | cancer | No | No |
| 109 | F | 7 | HepG2 + IL6 | cancer | No | No |
| 109 | G | 7 | Universal | unknown | Yes | No |
| 109 | H | 7 | SK-HEP-1 | cancer | No | No |
| 109 | A | 8 | ASPC1 | cancer | No | No |
| 109 | B | 8 | A-172 | cancer | No | No |
| 109 | C | 8 | A-172 cytoplasmic | cancer | Yes | No |
| 109 | D | 8 | U-138 MG | unknown | No | No |
| 109 | E | 8 | U-373 MG | cancer | Yes | No |
| 109 | F | 8 | BeWo | cancer | No | No |
| 109 | G | 8 | HeLa 229 | cancer | No | No |
| 109 | H | 8 | ME180 | cancer | Yes | No |
| 109 | A | 9 | PC-3 | cancer | Yes | No |
| 109 | B | 9 | A-549 | cancer | Yes | No |
| 109 | C | 9 | MRC-5 | normal | Yes | No |
| 109 | D | 9 | NCI-H69 | cancer | No | No |
| 109 | E | 9 | Sk-Lu-1 | cancer | Yes | No |
| 109 | F | 9 | WI38 | normal | Yes | No |
| 109 | G | 9 | SW480 | cancer | Maybe | No |
| 109 | H | 9 | A-431 | cancer | No | No |
| 109 | A | 10 | C32 | cancer | No | No |
| 109 | B | 10 | G-361 | cancer | Yes | No |
| 109 | C | 10 | HaCat | normal | No | No |
| 109 | D | 10 | Hs 294T | cancer | Yes | No |
| 109 | E | 10 | HT-144 | cancer | Yes | No |
| 109 | F | 10 | Malme 3M | cancer | Yes | No |
| 109 | G | 10 | Sk-MEL-2 | Cancer | Yes | No |
| 109 | H | 10 | WM-115 | unknown | No | No |
| 109 | A | 11 | HRCE | normal | No | No |
| 109 | B | 11 | 293 | disease | No | No |
| 109 | C | 11 | HRE | normal | No | No |
| 109 | D | 11 | HepSMCV | normal | No | No |
| 109 | E | 11 | HIAEC | normal | No | No |
| 109 | F | 11 | HMVEC-d Ad | normal | No | No |
| 109 | G | 11 | HMVEC-L | normal | Yes | No |
| 109 | H | 11 | NHAC-Kn | normal | No | No |
| 109 | A | 12 | NHEM-Neo | normal | No | No |
| 109 | B | 12 | SpSMC | normal | No | No |
| 109 | C | 12 | UASMC | normal | No | No |
| 109 | D | 12 | USMC | normal | No | No |
| 110 | A | 1 | Heart | disease | No | No |
| 110 | C | 1 | Heart | normal | No | No |
| 110 | D | 1 | Heart | normal | No | No |
| 110 | F | 1 | Heart | Normal | No | No |
| 110 | H | 1 | Heart (LV) | disease | Yes | No |
| 110 | A | 2 | Heart (LV) | disease | No | No |
| 110 | B | 2 | Heart (LV) | disease | Yes | No |
| 110 | C | 2 | Heart (LV) | disease | No | No |
| 110 | D | 2 | Heart (LV) | disease | No | No |
| 110 | E | 2 | Heart (LV) | disease | No | No |
| 110 | F | 2 | Heart (LV) | normal | Yes | No |
| 110 | G | 2 | Heart (LV) | normal | No | No |
| 110 | H | 2 | Heart (RV) | disease | No | No |
| 110 | B | 3 | Heart (V) | disease | No | No |
| 110 | C | 3 | Heart (atrium) | normal | No | No |
| 110 | A | 5 | Brain | cancer | No | No |
| 110 | B | 5 | Brain | cancer | Yes | No |
| 110 | C | 5 | Brain | cancer | No | No |
| 110 | D | 5 | Brain | cancer | Yes | No |
| 110 | E | 5 | Brain | cancer | No | No |
| 110 | F | 5 | Brain | cancer | No | No |
| 110 | G | 5 | Brain | cancer | No | No |
| 110 | H | 5 | Brain | cancer | No | No |
| 110 | A | 6 | Brain | normal | No | No |
| 110 | B | 6 | Brain | normal | Yes | Yes |
| 110 | C | 6 | Brain | normal | Yes | Yes |
| 110 | D | 6 | Brain | normal | Yes | Yes |
| 110 | E | 6 | Brain | normal | No | No |
| 111 | A | 1 | Caco-2, diff. cell line | cancer | No | No |
| 111 | B | 1 | Colon | cancer | No | No |
| 111 | C | 1 | Colon | cancer | Yes | No |
| 111 | D | 1 | Colon | cancer | No | No |
| 111 | E | 1 | Colon | cancer | Yes | No |
| 111 | F | 1 | Colon | cancer | No | No |
| 111 | G | 1 | Colon | cancer | No | No |
| 111 | H | 1 | Colon | cancer | No | No |
| 111 | A | 2 | Colon | cancer | No | No |
| 111 | B | 2 | Colon | cancer | Yes | No |
| 111 | C | 2 | Colon | cancer | No | No |
| 111 | D | 2 | Colon | cancer | No | No |
| 111 | E | 2 | Colon | cancer | No | No |
| 111 | F | 2 | Colon | disease | No | No |
| 111 | G | 2 | Colon | disease | Yes | No |
| 111 | H | 2 | Colon | disease | No | No |
| 111 | A | 3 | Colon | normal | No | No |
| 111 | B | 3 | Colon | normal | No | No |
| 111 | C | 3 | Colon | normal | Yes | No |
| 111 | D | 3 | Colon | normal | No | No |
| 111 | E | 3 | Colon | normal | No | No |
| 111 | F | 3 | Colon | normal | No | No |
| 111 | G | 3 | Colon | normal | No | No |
| 111 | H | 3 | Colon | normal | No | No |
| 111 | A | 4 | Colon | normal | No | No |
| 111 | B | 4 | Colon | normal | No | No |
| 111 | C | 4 | Esophagus | cancer | No | No |
| 111 | D | 4 | Esophagus | cancer | No | No |
| 111 | E | 4 | Esophagus | cancer | No | No |
| 111 | F | 4 | Esophagus | cancer | No | No |
| 111 | G | 4 | Esophagus | normal | Yes | No |
| 111 | H | 4 | Esophagus | normal | No | No |
| 111 | A | 5 | Esophagus | normal | No | No |
| 111 | B | 5 | Esophagus | no info | No | No |
| 111 | C | 5 | Liver | cancer | No | No |
| 111 | D | 5 | Liver | cancer | No | No |
| 111 | E | 5 | Liver | normal | No | No |
| 111 | F | 5 | Liver | normal | No | No |
| 111 | G | 5 | Parotid gland | cancer | No | No |
| 111 | H | 5 | Parotid gland | cancer | No | Yes |
| 111 | A | 6 | Parotid Gland | cancer | No | No |
| 111 | B | 6 | Parotid gland | cancer | No | No |
| 111 | C | 6 | Parotid gland | cancer | No | No |
| 111 | D | 6 | Parotid gland | cancer | No | No |
| 111 | E | 6 | Parotid gland | cancer | No | No |
| 111 | F | 6 | Parotid gland | cancer | No | No |
| 111 | G | 6 | Parotid gland | cancer | Yes | No |
| 111 | H | 6 | Parotid gland | disease | Yes | No |
| 111 | A | 7 | Parotid gland | normal | No | No |
| 111 | B | 7 | Parotid gland | normal | No | No |
| 111 | C | 7 | Parotid gland | normal | No | No |
| 111 | D | 7 | Salivary gland | cancer | No | No |
| 111 | E | 7 | Salivary gland | cancer | No | No |
| 111 | F | 7 | Small intestine | cancer | No | No |
| 111 | G | 7 | Small intestine | cancer | Yes | No |
| 111 | H | 7 | Small intestine | cancer | Yes | No |
| 111 | A | 8 | Small intestine | cancer | No | No |
| 111 | B | 8 | Small intestine | cancer | Maybe | No |
| 111 | C | 8 | Small intestine | cancer | No | No |
| 111 | D | 8 | Small intestine | cancer | Yes | No |
| 111 | E | 8 | Small intestine | cancer | No | No |
| 111 | F | 8 | Small intestine | cancer | No | No |
| 111 | G | 8 | Small intestine | disease | Yes | No |
| 111 | H | 8 | Small intestine | disease | No | No |
| 111 | A | 9 | Small intestine | normal | No | No |
| 111 | B | 9 | Small intestine | normal | Yes | No |
| 111 | C | 9 | Small intestine | normal | No | No |
| 111 | D | 9 | Small intestine | normal | No | No |
| 111 | E | 9 | Small intestine | normal | No | No |
| 111 | F | 9 | Small intestine | normal | No | No |
| 111 | G | 9 | Stomach | cancer | Yes | No |
| 111 | H | 9 | Stomach | cancer | Yes | No |
| 111 | A | 10 | Stomach | cancer | No | No |
| 111 | B | 10 | Stomach | cancer | No | No |
| 111 | C | 10 | Stomach | cancer | Yes | No |
| 111 | D | 10 | Stomach | cancer | No | No |
| 111 | E | 10 | Stomach | cancer | Yes | No |
| 111 | F | 10 | Stomach | cancer | Yes | No |
| 111 | G | 10 | Stomach | cancer | No | No |
| 111 | H | 10 | Stomach | cancer | No | No |
| 111 | A | 11 | Stomach | disease | No | No |
| 111 | B | 11 | Stomach | normal | No | No |
| 111 | C | 11 | Stomach | normal | No | No |
| 111 | D | 11 | Stomach | normal | No | No |
| 111 | E | 11 | Stomach | normal | No | No |
| 111 | F | 11 | Stomach | normal | No | No |
| 111 | G | 11 | Stomach | cancer | No | No |
| 111 | H | 11 | Stomach | normal | No | No |
| 111 | A | 12 | Stomach | normal | No | No |
| 111 | B | 12 | Stomach | normal | No | No |
| 111 | C | 12 | Stomach | normal | No | No |
| 111 | D | 12 | Stomach | normal | No | No |
| 112 | A | 1 | Adrenal | cancer | No | No |
| 112 | B | 1 | Adrenal | normal | No | No |
| 112 | C | 1 | Adrenal | normal | Yes | No |
| 112 | D | 1 | Adrenal | normal | No | No |
| 112 | E | 1 | Adrenal | normal | No | No |
| 112 | H | 1 | Adrenal | normal | Yes | No |
| 112 | A | 2 | Pancreas | cancer | Yes | No |
| 112 | B | 2 | Pancreas | cancer | No | No |
| 112 | C | 2 | Pancreas | cancer | Yes | No |
| 112 | D | 2 | Pancreas | cancer | No | No |
| 112 | E | 2 | Pancreas | disease | No | No |
| 112 | F | 2 | Pancreas | disease | Yes | No |
| 112 | G | 2 | Pancreas | normal | No | No |
| 112 | H | 2 | Pancreas | normal | No | No |
| 112 | A | 3 | Pancreas | normal | No | No |
| 112 | B | 3 | Pancreas | normal | No | No |
| 112 | C | 3 | Pancreas | normal | No | No |
| 112 | D | 3 | Pancreas | normal | Yes | No |
| 112 | E | 3 | Pancreas | normal | Yes | No |
| 112 | F | 3 | Pancreas | normal | No | No |
| 112 | G | 3 | Pancreas | normal | No | No |
| 112 | H | 3 | Pancreas | normal | No | No |
| 112 | A | 4 | Pancreas | normal | No | No |
| 112 | B | 4 | Pancreas | normal | No | No |
| 112 | D | 4 | Pancreas | normal | No | No |
| 112 | E | 4 | Pancreas | normal | No | No |
| 112 | F | 4 | Thyroid | cancer | Yes | No |
| 112 | G | 4 | Thyroid | cancer | No | No |
| 112 | H | 4 | Thyroid | cancer | Yes | No |
| 112 | A | 5 | Thyroid | cancer | No | No |
| 112 | B | 5 | Thyroid | cancer | Yes | No |
| 112 | C | 5 | Thyroid | disease | No | No |
| 112 | D | 5 | Thyroid | disease | No | No |
| 112 | E | 5 | Thyroid | no info | Yes | No |
| 112 | F | 5 | Thyroid | normal | Yes | No |
| 112 | G | 5 | Thyroid | normal | Yes | No |
| 112 | H | 5 | Thyroid | normal | No | No |
| 113 | A | 1 | Lymph node | cancer | No | No |
| 113 | B | 1 | Lymph node | cancer | No | No |
| 113 | C | 1 | Lymph node | cancer | No | No |
| 113 | D | 1 | Lymph node | normal | No | No |
| 113 | E | 1 | Lymph node | normal | No | No |
| 113 | F | 1 | Lymph node | normal | No | No |
| 113 | G | 1 | Lymphoma | cancer | No | No |
| 113 | H | 1 | Lymphoma | cancer | No | No |
| 113 | A | 2 | Lymphoma | cancer | Yes | No |
| 113 | B | 2 | Lymphoma | cancer | Yes | No |
| 113 | C | 2 | Spleen | normal | No | No |
| 113 | A | 4 | Bone | cancer | No | No |
| 113 | B | 4 | Bone | cancer | No | No |
| 113 | D | 4 | Bone (femur) | cancer | No | No |
| 113 | E | 4 | Bone (Sarc.) | cancer | Yes | No |
| 113 | F | 4 | Bone Femur | cancer | No | No |
| 113 | G | 4 | Bone marrow | normal | Maybe | No |
| 113 | A | 5 | Muscle | cancer | No | No |
| 113 | B | 5 | Muscle | disease | No | No |
| 113 | D | 5 | Muscle | normal | Yes | No |
| 113 | F | 5 | Muscle | normal | No | No |
| 113 | G | 5 | Muscle | normal | No | No |
| 113 | H | 5 | Muscle | normal | Yes | No |
| 113 | A | 6 | Muscle | normal | No | No |
| 113 | B | 6 | Muscle | normal | No | No |
| 113 | D | 6 | Muscle | normal | No | No |
| 113 | E | 6 | Skin | normal | No | No |
| 113 | F | 6 | Skin | normal | No | No |
| 113 | G | 6 | Skin | normal | Maybe | No |
| 113 | A | 7 | Skin | normal | Not | No |
| | | | | | determinable | |
| 113 | B | 7 | Skin | normal | No | No |
| 114 | A | 1 | Bladder | cancer | Yes | No |
| 114 | B | 1 | Bladder | normal | Yes | No |
| 114 | C | 1 | Kidney | cancer | Maybe | No |
| 114 | D | 1 | Kidney | cancer | No | No |
| 114 | E | 1 | Kidney | cancer | No | No |
| 114 | F | 1 | Kidney | cancer | No | No |
| 114 | G | 1 | Kidney | cancer | No | No |
| 114 | H | 1 | Kidney | cancer | Maybe | No |
| 114 | A | 2 | Kidney | cancer | No | No |
| 114 | B | 2 | Kidney | disease | Maybe | No |
| 114 | C | 2 | Kidney | disease | No | No |
| 114 | D | 2 | Kidney | disease | Yes | No |
| 114 | E | 2 | Kidney | disease | No | No |
| 114 | F | 2 | Kidney | normal | No | No |
| 114 | G | 2 | Kidney | normal | Maybe | No |
| 114 | H | 2 | Kidney | normal | No | No |
| 114 | A | 3 | Kidney | normal | No | No |
| 114 | B | 3 | Kidney | normal | No | No |
| 114 | C | 3 | Kidney | normal | No | No |
| 114 | D | 3 | Kidney | normal | No | No |
| 114 | E | 3 | Kidney | normal | No | No |
| 114 | F | 3 | Kidney | normal | No | No |
| 114 | G | 3 | Kidney | normal | No | No |
| 114 | A | 5 | Prostate | disease | No | No |
| 114 | B | 5 | Prostate | normal | Yes | Yes |
| 114 | C | 5 | Prostate Epithelia | cancer | Yes | No |
| 114 | D | 5 | Testis | cancer | Yes | No |
| 114 | E | 5 | Testis | cancer | Yes | No |
| 114 | F | 5 | Testis | normal | No | No |
| 114 | G | 5 | Testis | normal | Yes | No |
| 114 | H | 5 | Testis | normal | Yes | Yes |
| 114 | A | 6 | Breast | cancer | No | No |
| 114 | B | 6 | Breast | cancer | Maybe | No |
| 114 | C | 6 | Breast | cancer | No | No |
| 114 | D | 6 | Breast | normal | Maybe | No |
| 114 | F | 6 | Endometrium | cancer | Yes | No |
| 114 | G | 6 | Endometrium | cancer | No | No |
| 114 | H | 6 | Endometrium | cancer | No | No |
| 114 | A | 7 | Endometrium | cancer | Yes | Yes |
| 114 | B | 7 | Endometrium | cancer | No | No |
| 114 | C | 7 | Endometrium | cancer | No | No |
| 114 | D | 7 | Endometrium | cancer | No | No |
| 114 | E | 7 | Endometrium | cancer | Yes | No |
| 114 | F | 7 | Mammary Gland | cancer | Maybe | No |
| 114 | C | 8 | Ovary | cancer | No | No |
| 114 | D | 8 | Ovary | cancer | No | No |
| 114 | E | 8 | Ovary | cancer | Yes | No |
| 114 | F | 8 | Ovary | normal | No | No |
| 114 | G | 8 | Ovary | normal | No | No |
| 114 | H | 8 | Ovary | normal | No | No |
| 114 | A | 9 | Ovary | normal | Yes | No |
| 114 | B | 9 | Ovary | cancer | No | No |
| 114 | C | 9 | Placenta | normal | No | No |
| 114 | D | 9 | Placenta | normal | No | No |
| 114 | E | 9 | Uterus | cancer | Yes | No |
| 114 | F | 9 | Uterus | cancer | No | No |
| 114 | G | 9 | Uterus | cancer | No | No |
| 114 | H | 9 | Uterus | cancer | No | No |
| 114 | A | 10 | Uterus | cancer | Maybe | No |
| 114 | B | 10 | uterus | cancer | No | No |
| 114 | C | 10 | Uterus | normal | Yes | No |
| 114 | D | 10 | Uterus | normal | No | No |
| 114 | E | 10 | Uterus | normal | No | No |
| 114 | F | 10 | Uterus | normal | No | No |
| 114 | G | 10 | Uterus | normal | No | No |
| 114 | H | 10 | Uterus | normal | No | No |
| 115 | A | 1 | Bronchus | normal | Yes | No |
| 115 | D | 1 | Lung | cancer | No | No |
| 115 | E | 1 | Lung | cancer | No | No |
| 115 | F | 1 | Lung | cancer | No | No |
| 115 | H | 1 | Lung | cancer | Yes | No |
| 115 | A | 2 | Lung | cancer | Yes | No |
| 115 | C | 2 | Lung | cancer | Yes | No |
| 115 | D | 2 | Lung | cancer | Yes | No |
| 115 | E | 2 | Lung | cancer | No | No |
| 115 | F | 2 | Lung | cancer | No | No |
| 115 | G | 2 | Lung | cancer | Yes | No |
| 115 | H | 2 | Lung | cancer | Yes | No |
| 115 | A | 3 | Lung | cancer | Yes | No |
| 115 | B | 3 | Lung | cancer | Yes | No |
| 115 | C | 3 | Lung | cancer | Yes | No |
| 115 | E | 3 | Lung | normal | No | No |
| 115 | F | 3 | Lung | normal | Yes | No |
| 115 | G | 3 | Lung | normal | Yes | No |
| 115 | H | 3 | Lung | normal | Yes | No |
| 115 | A | 4 | Lung | normal | No | No |
| 115 | B | 4 | Lung | normal | No | No |
| 115 | C | 4 | Lung | normal | No | No |
| 115 | D | 4 | Lung | normal | No | No |
| 115 | E | 4 | Lung | normal | No | No |
| 115 | F | 4 | Lung | normal | No | No |
| 115 | G | 4 | Lung | normal | No | No |
| 115 | H | 4 | Lung | normal | Yes | No |
| 115 | A | 5 | Lung | normal | Not | No |
| | | | | | determinable | |
| 115 | B | 5 | Lung | normal | Yes | No |
| 115 | C | 5 | Lung | normal | No | No |
| 115 | D | 5 | Lung | normal | No | No |
| 115 | F | 5 | Lung | cancer | No | No |
| 115 | G | 5 | Trachea | normal | Yes | Yes |
| 116 | A | 1 | CD34+ | health | No | No |
| | | | | pending | | |
| 116 | C | 1 | CD19+ | normal | No | No |
| 116 | E | 1 | CD19+ resting | normal | No | No |
| 116 | G | 1 | CD19+ | unknown | No | No |
| 116 | A | 2 | CD19+ antiIgM/IL4 4 hr | unknown | No | No |
| 116 | C | 2 | CD19+ antiIgM/IL4 24 hr | unknown | No | No |
| 116 | E | 2 | CD19+ antiCD40 4 hr | unknown | No | No |
| 116 | G | 2 | CD19+ antiCD40 24 hr | unknown | No | No |
| 116 | A | 3 | monocytes | normal | No | No |
| 116 | C | 3 | CD14+ | unknown | No | No |
| 116 | E | 3 | CD14+ PMA/IONO. 4 hr | unknown | No | No |
| 116 | G | 3 | CD14+ PMA/IONO 24 hr | unknown | No | No |
| 116 | A | 4 | CD14+ gIFN 4 hr | unknown | No | No |
| 116 | C | 4 | CD14+ gIFN 24 hr | unknown | No | No |
| 116 | E | 4 | CD14+ | normal | No | No |
| 116 | G | 4 | CD14+ gIFN, LPS | normal | No | No |
| 116 | A | 5 | CD3+) | unknown | No | No |
| 116 | C | 5 | CD3+ PMA/IONO 4 hr | unknown | No | No |
| 116 | E | 5 | CD3+ PMA/IONO 24 hr | unknown | Yes | No |
| 116 | G | 5 | CD3+ antiCD3 4 hr | unknown | No | No |
| 116 | A | 6 | CD3+ antiCD3 24 hr | unknown | Yes | No |
| 116 | C | 6 | CD4+ | normal | No | No |
| 116 | E | 6 | NK resting 24 hr | normal | No | No |
| 116 | G | 6 | NK PMA/IONO 24 hr | normal | Yes | No |
| 116 | A | 7 | NK | normal | Yes | No |
| 116 | C | 7 | NK PMA, IONO, 12, 14, 20, 24 hours | normal | Yes | No |
| 116 | E | 7 | DC (CD14+, GMCSF, IL4 for 4, 5 or 6 d) | normal | No | No |
| 116 | G | 7 | DC (CD14+, GMCSF, IL4 for 4, 5 or 6 d) | normal | No | No |
| | | | TNFa, CD40L, LPS, polyIC, 4 & 20 hr | | | |
| 116 | A | 8 | plasmacytoid DC, (BDCA2+ from PBMC) | normal | No | No |
| | | | GMCSF, Flt3L, CpG 4, 24 hours | | | |
| 116 | C | 8 | MLR rest T = 0 | normal | No | No |
| 116 | E | 8 | MLR rest. 24 hr | normal | No | No |
| 116 | G | 8 | MLR 2.5 hr gIFN 50 ng/ml | normal | Maybe | No |
| 116 | A | 9 | MLR 24 hr gIFN 50 ng/ml | normal | Yes | No |
| 116 | C | 9 | MLR 48 hr gIFN 50 ng/ml | normal | No | No |
| 116 | E | 9 | Tonsil, inflamed | diseased | No | No |
| 117 | A | 1 | K562 | cancer | No | No |
| 117 | C | 1 | CCRF-CEM | cancer | No | No |
| 117 | E | 1 | HUT 78 | cancer | No | No |
| 117 | G | 1 | MV-4-11 | cancer | No | No |
| 117 | A | 2 | Raji | cancer | Yes | No |
| 117 | C | 2 | Ramos | cancer | No | No |
| 117 | E | 2 | Bjab | cancer | No | No |
| 117 | G | 2 | HS Sultan | cancer | No | No |
| 117 | A | 3 | DOHH-2 | cancer | No | No |
| 117 | C | 3 | WSU-NHL | cancer | No | No |
| 117 | E | 3 | RPMI1788 | normal | Yes | No |
| 117 | G | 3 | ARH-77 | cancer | No | No |
| 117 | A | 4 | Granta 519 | cancer | Yes | No |
| 117 | C | 4 | RL | cancer | No | No |
| 117 | E | 4 | RPMI 8226 | cancer | Yes | No |
| 117 | G | 4 | 697 | cancer | Yes | No |
| 117 | A | 5 | REH | cancer | Yes | No |
| 117 | C | 5 | TF-1 | cancer | Yes | No |
| 117 | E | 5 | HEL92.1.7 | cancer | Maybe | No |
| 117 | G | 5 | L363 | cancer | Yes | No |
| 117 | A | 6 | KG-1 | cancer | Yes | No |
| 117 | C | 6 | CESS | cancer | No | No |
| 117 | E | 6 | OPM-2 | cancer | No | No |
| 117 | G | 6 | TrBMEC | normal | Yes | No |
| 117 | A | 7 | THP1 | cancer | No | No |
| 117 | C | 7 | THP1 LPS/gIFN | cancer | Yes | No |
| 117 | E | 7 | U-937 | cancer | Yes | No |
| 117 | G | 7 | U-937 PMA/iono | cancer | Yes | No |
| 117 | A | 8 | U-937, untreated | cancer | Yes | No |
| 117 | C | 8 | U-937 PMA 12 hr | cancer | Yes | No |
| 117 | E | 8 | U-937 PMA 36 hr | cancer | Yes | No |
| 117 | G | 8 | U937 | cancer | Yes | No |
| 117 | A | 9 | HL-60 PMA 12 hr | cancer | Yes | No |
| 117 | C | 9 | HL-60 PMA 36 hr | cancer | No | No |
| 117 | E | 9 | HL-60 PMA 96 hr | cancer | No | No |
| 117 | G | 9 | HL-60 VitD3 36 hr | cancer | No | No |
| 117 | A | 10 | HL-60 ret. Acid pooled 12, 36, 96 hr | cancer | No | No |
| 117 | C | 10 | HL-60 butyric acid, pooled 12, 36, 96 hr | cancer | Yes | No |
| 117 | E | 10 | HL-60 DMSO 12 hr RT = yes | cancer | No | No |
| 117 | G | 10 | HL-60 DMSO 96 hr RT = yes | cancer | No | No |
| 117 | A | 11 | HL-60 RT = yes | cancer | No | No |
| 117 | C | 11 | HL-60 PMA RT = yes | cancer | Yes | No |
| 117 | E | 11 | HL-60 DMSO RT = yes | cancer | No | No |
| 117 | G | 11 | HL-60 butyrate RT = yes | cancer | No | No |
| 117 | A | 12 | HL-60 PMA RT = yes | cancer | No | No |

### Example 8

### Tissue Distribution in cDNA Library Panels Using PCR

A panel of DNAs from cDNA libraries made in-house was screened for ztnf11 expression using PCR. The panel contained 45 DNA samples from cDNA libraries made from various human tissues (normal, cancer, and diseased) and resting or stimulated cell lines. The in-house cDNA libraries were QC tested by PCR with vector oligos for average insert size, PCR for alpha tubulin or G3PDH for full length cDNA using 5′ vector oligo and 3′ gene specific oligo and sequencing for ribosomal or mitochondrial DNA contamination. The panel was set up in a 96-well format that included a 100 pg human genomic DNA (BD Biosciences Clontech, Palo Alto, Calif.) positive control sample. Each well contained 5 ul of cDNA library DNA and 8.0 ul of water. The PCR reactions were set up using 0.5 μl of 20 uM each of oligos ZC47124 (SEQ ID NO:20) and ZC47127 (SEQ ID NO:21), 2.5 ul 10× buffer and 0.5 ul Advantage 2 cDNA polymerase mix (BD Biosciences Clontech, Palo Alto, Calif.), 1 ul 2.5 mM dNTP mix (Applied Biosystems, Foster City, Calif.), 10% DMSO (Sigma, St. Louis, Mo.) and 1× Rediload dye (Invitrogen, Carlsbad, Calif.) in a final volume of 27.5 ul. The amplification was carried out as follows: 1 cycle at 94° C. for 2 minutes, 35 cycles of 94° C. for 30 seconds, 64° C. for 30 seconds and 72° C. for 45 seconds, followed by 1 cycle at 72° C. for 5 minutes. About 10 μl of the PCR reaction product was subjected to standard agarose gel electrophoresis using a 4% agarose gel. There is a long (×1) and short (×2) form of ztnf11. The oligos can pick up both forms. They differ by 564 bp. The long form is 669 bp and the short form is 105 bp. See Table 6 below for expression profile for both forms and tissues screened. The genomic band is 861 bp in size.

The results indicate that ztnf11×1 and ztnf11×2 are not highly represented in these cDNA libraries. Positives for ztnf11×1 occur only in the pancreas and U-937 libraries, which is consistent with pancreas and U-937 positive results in other PCR assays. Ztnf11×2 is only positive in the pancreas library.

TABLE 6

| | | Ztnf11x1 |
|---|---|---|
| cDNA's | Source | Long Form |
| HL-60 vitD 12, 3, 96 hrs | in-house | No |
| HL-60 Ret. Acid 12, 3, 96 hrs | in-house | No |
| HL-60 Butyric Acid 12, 3, | in-house | No |
| 96 hrs | | |
| THP-1 #2 IFNg 13, 39 hrs | in-house | No |
| HT-29 | in-house | No |
| Fetal brain | in-house | No |
| Brain | in-house | No |
| Spinal cord | in-house | No |
| Pancreas | in-house | Yes |
| Islet | in-house | No |
| Pituitary | in-house | No |
| Kidney | in-house | No |
| Thyroid | in-house | No |
| Fetal thymus | in-house | No |
| Prostate SMC | in-house | No |
| Prostate 0.5-1.6 KB | in-house | No |
| Prostate >1.6 KB | in-house | No |
| Fetal liver | in-house | No |
| Tonsil | in-house | No |
| Inflamed tonsil | in-house | No |
| HaCat | in-house | No |
| KG-1 | in-house | No |
| CaCO-2 | in-house | No |
| SKLU-1 | in-house | No |
| REH | in-house | No |
| RPMI (B-cells) | in-house | No |
| HL60 + PMA | in-house | No |
| HL60 + PMA | in-house | No |
| K562 | in-house | No |
| THP-1 | in-house | No |
| THP-1 | in-house | No |
| U-937 | in-house | Yes |
| U-937 PMA 12, 36 hrs | in-house | No |
| PBMC-1 | in-house | No |
| PBMC-2 | in-house | No |
| CD4+ | in-house | No |
| CD4+ | in-house | No |
| CD3+ | in-house | No |
| CD19+ | in-house | No |
| CD14+ | in-house | No |
| CD14+ IFNg/LPS | in-house | No |
| Dendritic Cell | in-house | No |
| Dendritic Cell, stim | in-house | No |
| NK PMA/IONO | in-house | No |
| | | Ztnf11x2 |
| cDNA's | Made | Short Form |
| HL-60 vitD 12, 3, 96 hrs | in-house | No |
| HL-60 Ret. Acid 12, 3, 96 hrs | in-house | No |
| HL-60 Butyric Acid 12, 3, | in-house | No |
| 96 hrs | | |
| THP-1 #2 IFNg 13, 39 hrs | in-house | No |
| HT-29 | in-house | No |
| Fetal brain | in-house | No |
| Brain | in-house | No |
| Spinal cord | in-house | No |
| Pancreas | in-house | Yes |
| Islet | in-house | No |
| Pituitary | in-house | No |
| Kidney | in-house | No |
| Thyroid | in-house | No |
| Fetal thymus | in-house | No |
| Prostate SMC | in-house | No |
| Prostate 0.5-1.6 KB | in-house | No |
| Prostate >1.6 KB | in-house | No |
| Fetal liver | in-house | No |
| Tonsil | in-house | No |
| Inflamed tonsil | in-house | No |
| HaCat | in-house | No |
| KG-1 | in-house | No |
| CaCO-2 | in-house | No |
| SKLU-1 | in-house | No |
| REH | in-house | No |
| RPMI (B-cells) | in-house | No |
| HL60 + PMA | in-house | No |
| HL60 + PMA | in-house | No |
| K562 | in-house | No |
| THP-1 | in-house | No |
| THP-1 | in-house | No |
| U-937 | in-house | No |
| U-937 PMA 12, 36 hrs | in-house | No |
| PBMC-1 | in-house | No |
| PBMC-2 | in-house | No |
| CD4+ | in-house | No |
| CD4+ | in-house | No |
| CD3+ | in-house | No |
| CD19+ | in-house | No |
| CD14+ | in-house | No |
| CD14+ IFNg/LPS | in-house | No |
| Dendritic Cell | in-house | No |
| Dendritic Cell, stim | in-house | No |
| NK PMA/IONO | in-house | No |

### Example 9

### Tissue Distribution in Blood Fraction Panel Using PCR

A panel of 1^{st} strand cDNAs from human cells and tissues was screened for ztnf11 expression using PCR. The panel was purchased from BD Bioscience (Palo Alto, Calif.) and contained 10 cDNA samples from various human blood cells and tissues. The 1^{st} strand cDNAs were QC tested by PCR with G3PDH control primers by BD BioScience (Palo Alto, Calif.). The panel was set up in a 96-well format that included 1 positive control sample, human thyroid 1^{st} strand cDNA. A dilution series was performed. Each well contained either 5 ul of cDNA and 8.0 ul of water, 1 ul of cDNA and 12.0 ul of water or 1 ul of a 1:5 dilution of cDNA and 12.0 ul water. The PCR reactions were set up using 0.5 μl of 20 uM each of oligos ZC47124 (SEQ ID NO:20) and ZC47127 (SEQ ID NO:21), 2.5 ul 10× buffer and 0.5 ul Advantage 2 cDNA polymerase mix (BD Biosciences Clontech, Palo Alto, Calif.), 1 ul 2.5 mM dNTP mix (Applied Biosystems, Foster City, Calif.), 10% DMSO (Sigma, St. Louis, Mo.) and 1× Rediload dye (Invitrogen, Carlsbad, Calif.) in a final volume of 27.5 ul. The amplification was carried out as follows: 1 cycle at 94° C. for 2 minutes, 35 cycles of 94° C. for 30 seconds, 64° C. for 30 seconds and 72° C. for 45 seconds, followed by 1 cycle at 72° C. for 5 minutes. About 10 g of the PCR reaction product was subjected to standard agarose gel electrophoresis using a 4% agarose gel. There is a long (×1) and short (×2) form of ztnf11. The oligos can pick up both forms. They differ by 564 bp. The long form is 669 bp and the short form is lO5bp. See Table 7 below for expression profile for both forms and tissues screened. The genomic band is 861 bp in size.

The results indicate that ztnf11×2 mRNA is not expressed in any of these peripheral blood fractions, while in contrast ztnf11×1 mRNA is expressed in many of these samples, including activated and resting CD4+ T-helper cells and CD19+ B-cells, resting CD8+ cytotoxic T-cells, mononuclear cells and possibly activated mononuclear cells. Resting CD14+ monocyte cells and activated CD8+ cytotoxic T-cells are negative for ztnf11×1 in this assay.

TABLE 7

| | | Ztnf11x1 |
|---|---|---|
| cDNA's | Source | Long Form |
| Activated CD4+ | BD Bioscience | Yes |
| Resting CD4+ | BD Bioscience | Yes |
| Activated CD8+ | BD Bioscience | No |
| Resting CD8+ | BD Bioscience | Yes |
| Resting CD14+ | BD Bioscience | No |
| Activated CD19+ | BD Bioscience | Yes |
| Resting CD19+ | BD Bioscience | Yes |
| Activated Mononuclear | BD Bioscience | Maybe |
| Mononuclear | BD Bioscience | Yes |
| Control placenta | BD Bioscience | Yes |
| | | Ztnf11x2 |
| cDNA's | Made | Short Form |
| Activated CD4+ | BD Bioscience | No |
| Resting CD4+ | BD Bioscience | No |
| Activated CD8+ | BD Bioscience | No |
| Resting CD8+ | BD Bioscience | No |
| Resting CD14+ | BD Bioscience | No |
| Activated CD19+ | BD Bioscience | No |
| Resting CD19+ | BD Bioscience | No |
| Activated Mononuclear | BD Bioscience | No |
| Mononuclear | BD Bioscience | No |
| Control placenta | BD Bioscience | No |

### Example 10

### Expression of ztnf11 on Northern Blots and Disease Profiling Arrays

Sense primer zc47124 (SEQ ID NO:20) and antisense primer zc47127 (SEQ ID NO:21) were used in a 25 ul PCR reaction to generate a 669 bp fragment for use on northern blots and disease arrays as follows: 2.5 ul 10× Advantage 2 buffer and 0.5 ul Advantage 2 polymerase mix (BD Biosciences, Clontech, Palo Alto, Calif.), 2.5 ul Redi-Load (Invitrogen, Carlsbad, Calif.), 1 ul 2.5 mM dNTPs (Applied Biosystems, Foster City, Calif.) 0.5 ul 20 uM each zc47124 and 47127, 2 ul first strand cDNA from pancreas (representing first strand cDNA from 100 ng starting total RNA), 10% DMSO (Sigma, St. Louis, Mo.), and H2O to 27.5 ul. Cycling conditions were 1 cycle at 94° C. 2′, 35 cycles at 94° C. 30″, 64° C. 30″ 72° C. 45″, followed by one cycle at 72° C. 5′, and a hold at 4° C. Reactions were run in an agarose gel and fragments were purified using Qiagen gel purification columns (Qiagen, Valencia, Calif.) according to the manufacturer's instructions. The fragment was quantitated by a spectrophotometer reading, and sequenced to confirm it as a ztnf11 subsequence. 36 ng of fragment was labeled using Prime-It II reagents (Stratagene, La Jolla, Calif.) according to the manufacturer's instructions, and separated from unincorporated nucleotides using an S-200 microspin column (Amersham, Piscataway, N.J.) according to the manufacturer's protocol. Blots to be probed with ztnf11 (Autoimmune and Blood Disease Profiling Arrays, Cancer Profiling Array II, Multiple Tissue Northern Blots I, II, and III, all from BD Biosciences, Clontech, Palo Alto, Calif., and one in-house blot with 1 ug/lane mRNA from immune related cell lines) were prehybridized overnight at 55° C. in ExpressHyb (BD Biosciences, Clontech Palo Alto, Calif.) in the presence of 100 ug/ml salmon sperm DNA (Stratagene, La Jolla, Calif.) and 6 ug/ml cot-I DNA (Invitrogen, Carlsbad, Calif.) which were boiled and snap-chilled prior to adding to the blots. Radiolabelled ztnf11, salmon sperm DNA and cot-1 DNA were mixed together and boiled 5′, followed by a snap chilling on ice. Final concentrations of the salmon sperm DNA and cot-1 DNA were as in the prehybridization step and the final concentration of radiolabelled ztnf11 was 1×106 cpm/ml. Blots were hybridized overnight in a roller oven at 55° C., then washed copiously at RT in 2×SSC, 0.1% SDS, with several buffer changes. Then washed at 65° C. in 0.1×SSC, 0.1% SDS, with two buffer changes. Blots were then exposed to film with intensifying screens for 7 days. The immune cell line blot and multiple tissue northern blots were then probed with a transferrin receptor probe, generated by PCR. The reaction was run in an agarose gel and the fragment were purified using Qiagen gel purification columns (Qiagen, Valencia, Calif.) according to the manufacturer's instructions. The fragment was quantitated by a spectrophotometer reading. The transferrin receptor fragment was labeled and used to probe the Multiple Tissue Northern Blots and the immune cell line northern blot as described above. Blots were exposed to film with intensifying screens for 8 days or 1 day.

Results of probing multiple tissue northern blots with ztnf 1l indicate that ztnf11 mRNA is generally rare with the exception of high expression in testis. Ztnf11 mRNA is observed in several cell lines on the immune cell line northern blot: Daudi, HL60, Jurkat, RPMI8226, and U-937. Daudi and RPMI 8226 are described as B-lymphoblast cell lines derived from Burkitt's lymphoma and myeloma, respectively. Jurkat is described as an acute lymphoblastic T-cell leukemia cell line, as is Molt-4, which was negative for ztnf11. And lastly, U-937, a monocyte cell line, was positive for ztnf11. It also appears that two transcript sizes can be ascribed to ztnf11, about 1.4 kb as seen in testis, and about 2kb as seen in Daudi, HL60, RPMI 8226, U-937 and very faintly in stomach.

The transferrin receptor control probing experiment shows the blots were of good quality and a low to moderately expressed control gene could be observed with a 1 or 8 day exposure. Additionally, in the Cancer Profiling Array, in some tumor types ztnf11 mRNA appears to be downregulated as compared to normal ztnf11 mRNA levels. This difference can be observed in stomach, kidney, liver, pancreas, small intestine, thyroid, and possibly rectum. In the Blood and Autoimmune Disease Profiling Arrays, ztnf11 levels are generally low, with no obvious correlation to disease conditions. Ztnf11 expression does appear slightly higher in the CD 19+ (B-cell) and mononuclear cell fractions of most patients, however, this observation could be due to several factors, including normalization procedures used on the disease profiling arrays.

### Example 11

### Tissue Distribution of Mouse cDNA Using PCR

A panel of DNAs from cDNA libraries and marathon cDNAs made in-house was screened for ztnf11 mouse expression using PCR. The panel contained 49 DNA samples from cDNA libraries and marathon cDNAs made from various mouse tissues (normal, cancer, and diseased) and resting or stimulated cell lines. The in-house cDNA libraries were QC tested by PCR with vector oligos for average insert size, PCR for alpha tubulin or G3PDH for full length cDNA, and sequencing for ribosomal or mitochondrial DNA contamination. The panel was QC tested by PCR with murine cathepsin z primers. The panel was set up in a 96-well format that included 1 ng mouse genomic DNA (BD Biosciences Clontech, Palo Alto, Calif.) positive control sample. Each well contained 17.5 ul of cDNA and water. The PCR reactions were set up using 0.5 μl of 20 uM each of oligos ZC47363 (SEQ ID NO:22) and ZC47364 (SEQ ID NO:23), 2.5 ul 10× buffer and 0.5 ul Advantage 2 cDNA polymerase mix (BD Biosciences Clontech, Palo Alto, Calif.), 1 ul 2.5 mM dNTP mix (Applied Biosystems, Foster City, Calif.), 10% DMSO (Sigma, St. Louis, Mo.) and 1× Rediload dye (Invitrogen, Carlsbad, Calif.) in a final volume of 27.5 ul. The amplification was carried out as follows: 1 cycle at 94° C. for 2 minutes, 35 cycles of 94° C. for 30 seconds, 61.9° C. for 30 seconds and 72° C. for 30 seconds, followed by 1 cycle at 72° C. for 5 minutes. About 10 μl of the PCR reaction was subjected to stantard agarose gel electrophoresis using a 4% agarose gel. The mouse ztnf11 PCR product is 289 bp, and any contaminating genomic DNA is distinguishable by a PCR product 542 bp in size.

The results of this expression profile, seen in Table 8 below, indicate that mouse ztnf11 is not highly represented in these cDNA samples. Positives for ztnf11 occur only in testis, skeletal muscle, skin and the Jakotay prostate cell line from p53−/− mice. In testis and skin, ztnf11 mRNA expression was quite high, mirroring the high expression in testis seen on northern blot, and by PCR on human skin-derived cell lines.

TABLE 8

| | | | | | Ztnf11 |
|---|---|---|---|---|---|
| Plate # | Row | Col. | Tissue/Cell Line | Biological System | mRNA |
| 108 | 1 | A | 229 | Skeletal | No |
| 108 | 1 | B | 7F2 | Skeletal | No |
| 108 | 1 | C | 7F2-Fat | Skeletal | No |
| 108 | 1 | D | Adipocytes-Amplified | Skeletal | No |
| 108 | 1 | E | alpha TC1.9 pancreas alpha cell | Digestive (and Endocrine) | No |
| | | | line | | |
| 108 | 1 | F | Brain | Nervous | No |
| 108 | 1 | G | Brain | Nervous | No |
| 108 | 1 | H | Brain | Nervous | No |
| 108 | 2 | A | Brain-Arrayed Library | Nervous | No |
| 108 | 2 | B | CCC4 osteoblast cell line | Skeletal | No |
| 108 | 2 | C | Spleen CD90+ (Amplified) T cells | Lymphatic | No |
| 108 | 2 | C | Spleen CD90+ (Amplified) T cells | Lymphatic | No |
| 108 | 2 | D | combined OC10B osteoblast cell | Skeletal | No |
| | | | line | | |
| 108 | 2 | E | Dendritic | PBLs | No |
| 108 | 2 | F | Embryo (Library) | Whole body | No |
| 108 | 2 | G | Heart | Cardiovascular (and | No |
| | | | | Endocrine) | |
| 108 | 2 | H | Heart | Cardiovascular (and | No |
| | | | | Endocrine) | |
| 108 | 3 | A | Kidney | Urinary (and Endocrine) | No |
| 108 | 3 | B | Kidney | Urinary (and Endocrine) | No |
| 108 | 3 | C | Kidney | Urinary (and Endocrine) | No |
| 108 | 3 | D | Liver | Digestive | No |
| 108 | 3 | E | Liver | Digestive | No |
| 108 | 3 | F | Lung | Respiratory | No |
| 108 | 3 | G | Lung | Respiratory | No |
| 108 | 3 | H | MEWt#2-Amplified | BAF3 transfected/Bone | No |
| | | | | Marrow | |
| 108 | 4 | A | p388D1 macrophage-like cell line | Lymphatic | No |
| 108 | 4 | B | Pancreas | Digestive (and Endocrine) | No |
| 108 | 4 | C | Placenta-Amplified | Female Reproductive (and | No |
| | | | | Endocrine) | |
| 108 | 4 | D | Placenta-Arrayed Library | Female Reproductive (and | No |
| | | | | Endocrine) | |
| 108 | 4 | E | Jakotay-Prostate Cell Line | Male Reproductive | Yes |
| 108 | 4 | F | Nelix-Prostate Cell Line | Male Reproductive | No |
| 108 | 4 | G | Paris-Prostate Cell Line | Male Reproductive | No |
| 108 | 4 | H | Torres-Prostate Cell Line | Male Reproductive | No |
| 108 | 5 | A | Tuvak-Prostate Cell Line | Male Reproductive | No |
| 108 | 5 | B | Salivary-Amplified | Digestive | No |
| 108 | 5 | C | Salivary-Arrayed Library | Digestive | No |
| 108 | 5 | D | Skeletal Muscle | Muscular | Maybe |
| 108 | 5 | E | Skin | Integumentary | Yes |
| 108 | 5 | F | Skin-Amplified | Integumentary | No |
| 108 | 5 | G | Small Intestine | Digestive (and Endocrine) | No |
| 108 | 5 | H | Smooth Muscle | Various | No |
| 108 | 6 | A | Smooth Muscle | Various | No |
| 108 | 6 | B | Spleen | Lymphatic | No |
| 108 | 6 | C | Spleen | Lymphatic | No |
| 108 | 6 | D | Stomach | Digestive (and Endocrine) | No |
| 108 | 6 | E | Testis | Male Reproductive (and | Yes |
| | | | | Endocrine) | |
| 108 | 6 | F | Testis-Amplified | Male Reproductive (and | Yes |
| | | | | Endocrine) | |
| 108 | 6 | G | Testis-Arrayed Library | Male Reproductive (and | Yes |
| | | | | Endocrine) | |
| 108 | 6 | H | Thymus | Lymphatic (and Endocrine) | No |
| 108 | 7 | A | Uterus | Female Reproductive (and | No |
| | | | | Endocrine) | |
| 108 | 7 | h | Genomic | | |

### Example 12

### Expression Construct for ztnf11×1

EST clone (clone image #5742860, MGC# 46279, ATCC, Manassas, Va.) was ordered, sequenced, and to be ztnf11. A gene construct of the clone was generated by digestion with EcoRI(GibCo-BRL, Invitrogen, Carlsbad, Calif.) and XbaI(Roche, Indianapolis, Ind.) digestion enzymes. The digestion reaction was subjected to standard agarose gel electrophoresis using a 1% agarose gel then purified using a Gel Extraction Kit (Qiagen, Chatsworth, Calif.) according to manufacturer's instructions. The Digested cDNA was ligated using T4 DNA liage and buffer (GibCo-BRL, Invitrogen, Carlsbad, Calif.). The ligation reaction was transformed into DH10B Top10 chemically competent One shot cells (Invitrogen, Carlsbad, Calif.) according to manufacturer's instructions. Transformants were screened by digestion and then submitted to sequencing. Sequence confirmed clones were ztnf11. The construct named ztnf11/pzp7P.

### Example 13

### Mammalian Expression Plasmids

An expression plasmid containing a polynucleotide encoding zTNF11, can be constructed via homologous recombination. A fragment of cDNA for example zTNF11 cDNA, is isolated by PCR using the polynucleotide sequence of SEQ ID NO:(Clonetrack #101384) with flanking regions at the 5′ and 3′ ends corresponding to the vector sequences flanking the zTNF11 insertion point. The primers ZC47213 and ZC47214 are shown in SEQ ID NOS:24 and 25, respectively.

The PCR reaction mixture is run on a 1% agarose gel and a band corresponding to the size of the insert is gel-extracted using a QIAquick™ Gel Extraction Kit (Qiagen, Valencia, Calif.). Plasmid pZMP21 is a mammalian expression vector containing an expression cassette having the MPSV promoter, multiple restriction sites for insertion of coding sequences, a stop codon, an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae. It is constructed from pZP9 (deposited at the American Type Culture Collection, 10801 University Boulevard, Manassas, Va. 20110-2209, under Accession No. 98668) with the yeast genetic elements taken from pRS316 (deposited at the American Type Culture Collection, 10801 University Boulevard, Manassas, Va. 20110-2209, under Accession No. 77145), an internal ribosome entry site (IRES) element from poliovirus, and the extracellular domain of CD8 truncated at the C-terminal end of the transmembrane domain. Plasmid pZMP21 was digested with BglII, and used for recombination with the PCR insert.

One hundred microliters of competent yeast (S. cerevisiae) cells are independently combined with 2 μl of the various DNA mixtures from above and transferred to a 0.2-cm electroporation cuvette. The yeast/DNA mixtures are electropulsed using power supply (BioRad Laboratories, Hercules, Calif.) settings of 0.75 kV (5 kV/cm), ¥ ohms, 25 μF. To each cuvette is added 400 μl of 1.2 M sorbitol, and the yeast is plated in two 300-μl aliquots onto two URA-D plates and incubated at 30° C. After about 48 hours, the Ura+ yeast transformants from a single plate are resuspended in 1 ml H2O and spun briefly to pellet the yeast cells. The cell pellet is resuspended in 1 ml of lysis buffer (2% Triton X-100, 1% SDS, 100 mM NaCl, 10 mM Tris, pH 8.0, 1 mM EDTA). Five hundred microliters of the lysis mixture is added to an Eppendorf tube containing 300 μl acid-washed glass beads and 200 μl phenol-chloroform, vortexed for 1 minute intervals two or three times, and spun for 5 minutes in an Eppendorf centrifuge at maximum speed. Three hundred microliters of the aqueous phase is transferred to a fresh tube, and the DNA is precipitated with 600 μl ethanol (EtOH), followed by centrifugation for 10 minutes at 4° C. The DNA pellet is resuspended in 30 μl H2O.

Transformation of electrocompetent E. coli host cells (Electromax DH10BÔ cells; obtained from Life Technologies, Inc., Gaithersburg, Md.) is done with 0.5-2 ml yeast DNA prep and 37 ml of cells. The cells are electropulsed at 1.7 kV, 25 μF, and 400 ohms. Following electroporation, 1 ml SOC (2% BactoÔ Tryptone (Difco, Detroit, Mich.), 0.5% yeast extract (Difco), 10 mM NaCl, 2.5 mM KCl, 10 mM MgCl2, 10 mM MgSO4, 20 mM glucose) is plated in 250-μl aliquots on four LB AMP plates (LB broth (Lennox), 1.8% BactoÔ Agar (Difco), 100 mg/L Ampicillin).

Individual clones harboring the correct expression construct for zTNF11 are identified by restriction digest to verify the presence of the zTNF11 insert and to confirm that the various DNA sequences have been joined correctly to one another. The inserts of positive clones are subjected to sequence analysis. Larger scale plasmid DNA is isolated using a commercially available kit (QIAGEN Plasmid Maxi Kit, Qiagen, Valencia, Calif.) according to manufacturer's instructions. The correct construct is designated zTNF11-nFLAG/pZMP21.

### Example 14

### Protein Production

Three sets of 200 kg of the zTNF11_NF construct were each digested with 200 units of Pvu I at 37° C. for three hours and then were precipitated with EPA and spun down in a 1.5 mL microfuge tube. The supernatant was decanted off the pellet, and the pellet was washed with 1 mL of 70% ethanol and allowed to incubate for 5 minutes at room temperature. The tube was spun in a microfuge for 10 minutes at 14,000 RPM and the supernatant was decanted off the pellet. The pellet was then resuspended in 750 μl of PF-CHO media in a sterile environment, allowed to incubate at 60° C. for 30 minutes, and was allowed to cool to room temperature. 5E6 APFDXB11 cells were spun down in each of three tubes and were resuspended using the DNA-media solution. The DNA/cell mixtures were placed in a 0.4 cm gap cuvette and electroporated using the following parameters: 950 μF, high capacitance, and 300 V. The contents of the cuvettes were then removed, pooled, and diluted to 25 mLs with PF-CHO media and placed in a 125 mL shake flask. The flask was placed in an incubator on a shaker at 37° C., 6% CO2, and shaking at 120 RPM.

The cell line was subjected to nutrient selection followed by step amplification to 200 nM methotrexate (MTX), and then to 1 mM MTX. Expression of secreted protein is confirmed by western blot, and the cell line is scaled-up for protein purification.

### Example 15

### Construction of ztnf11-MBP Fusion Expression Vector pTAP170/ztnf11

An expression plasmid containing a polynucleotide encoding part of the human ztnf11 fused N-terminally to maltose binding protein (MBP) was constructed via homologous recombination. A fragment of human ztnf11 cDNA (SEQ ID NO:26) was isolated using PCR. Two primers were used in the production of the human ztnf11 fragment in a PCR reaction: (1) Primer zc47357 (SEQ ID NO:27), containing 34 bp of the vector flanking sequence and 24 bp corresponding to the amino terminus of the human ztnf11, and (2) primer ZC47358 (SEQ ID NO:28), containing 25 bp of the 3′ end corresponding to the flanking vector sequence and 24 bp corresponding to the carboxyl terminus of the human ztnf11. The PCR reaction conditions were as follows: The PCR amplification reaction condition is as follows: 1 cycle, 95° C., 2 minutes; 30 cycles, 95 ° C., 30 seconds, followed by 62 ° C., 1 minute, followed by 72° C., 2.5 minutes; 1 cycle, 72° C., 10 minutes. Each of four 25 μl PCR reaction were run on a 1.2% agarose gel and the expected band of approximately 836 bp fragment was seen. The 836 bp band was excised from the gel and purified using QIAquick Gel Extraction Kit (Qiagen, Cat. No. 28704). according to manufacturer's directions. DNA was eluted from the spin column in 30 ml of Elution Buffer B. Ten ml of purified PCR product was used for recombining into the SmaI cut recipient vector pTAP170 to produce the construct encoding the MBP-human ztnf11 fusion, as described below.

Plasmid pTAP170 was derived from the plasmids pRS316 and pMAL-c2. The plasmid pRS316 is a Saccharomyces cerevisiae shuttle vector (Hieter P. and Sikorski, R., Genetics 122:19-27, 1989). pMAL-C2 (NEB) is an E. coli expression plasmid. It carries the tac promoter driving MalE (gene encoding MBP) followed by a His tag, a thrombin cleavage site, a cloning site, and the rrnB terminator. The vector pTAP170 was constructed using yeast homologous recombination. 100 ng of EcoR1 cut pMAL-c2 was recombined with 1 mg Pvu1 cut pRS316, 1 mg linker, and 1 mg Sca1/EcoR1 cut pRS316. The linker consisted of oligos zc19,372 (SEQ ID NO:29) (100 pmole): zc19,351 (SEQ ID NO:30) (1 pmole) : zc19,352 (SEQ ID NO:31) (1 pmole), and zc19,371 (SEQ ID NO:32) (100 pmole) combined in a PCR reaction. Conditions were as follows: 10 cycles of 94° C. for 30 seconds, 50° C. for 30 seconds, and 72° C. for 30 seconds; followed by 4° C. soak. PCR products were concentrated via 100% ethanol precipitation.

One hundred microliters of competent yeast cells (S. cerevisiae) were combined with 10 μl of a mixture containing approximately 1 μg of the human ztnf11 insert, and 100 ng of SmaI digested pTAP170 vector, and transferred to a 0.2 cm electroporation cuvette. The yeast/DNA mixture was electropulsed at 0.75 kV (5 kV/cm), infinite ohms, 25 μF. To each cuvette was added 600 μl of 1.2 M sorbitol. The yeast was then plated in two 300 μl aliquots onto two-URA D plates and incubated at 30° C.

After about 48 hours, the Ura+ yeast transformants from a single plate were resuspended in 1 ml H2O and spun briefly to pellet the yeast cells. The cell pellet was resuspended in 1 ml of lysis buffer (2% Triton X-100, 1% SDS, 100 mM NaCl, 10 mM Tris, pH 8.0, 1 mM EDTA). Five hundred microliters of the lysis mixture was added to an Eppendorf tube containing 300 μl acid washed glass beads and 500 μl phenol-chloroform, vortexed for 1 minute intervals two or three times, followed by a 5 minute spin in a Eppendorf centrifuge at maximum speed. Three hundred microliters of the aqueous phase was transferred to a fresh tube, and the DNA precipitated with 600 μl ethanol (EtOH), followed by centrifugation for 10 minutes at 4° C. The DNA pellet was resuspended in 100 μl H2O.

Transformation of electrocompetent E. coli cells (DH10B, Invitrogen) was done with 1 ml yeast DNA prep and 40 ml of DH10B cells. The cells were electropulsed at 2.5 kV, 25 mF and 400 ohms. Following electroporation, 1.0 ml SOC (2% BactoI Tryptone (Difco, Detroit, Mich.), 0.5% yeast extract (Difco), 10 mM NaCl, 2.5 mM KCl, 10 mM MgCl2, 10 mM MgSO4, 20 mM glucose) was added to the cells. After incubation for 30 minutes at 37° C., the cells were plated in one aliquot on LB Kan plates (LB broth (Lennox), 1.8% Bactoa Agar (Difco), 30 mg/L kanamycin).

Individual clones harboring the correct expression construct for human ztnf11 were identified by colony PCR and sequence verified. Colony PCR reaction conditions were as follows: 1 cycle, 95° C., 5 minutes; 30 cycles, 95° C., 15 seconds, followed by 55° C., 0.30 seconds, followed by 68° C., 30 seconds; 1 cycle, 68° C, 2 minutes. Ten μl of each of forty eight 25 μl PCR reaction were run on a 1.2% agarose gel and the expected band of approximately 836 bp fragment was seen.

Double-stranded sequence of the two colony PCR positive clones were determined using the ABI PRISM BigDye Terminator v2.0 Cycle Sequencing Kit (Applied Biosystems, Foster City, Calif.). Sequencing reactions were purified using EdgeBioSystems Centriflex Gel Filtration Cartridges (Gaithersburg, Md.) and run on an ABI PRISM 377 DNA Sequencer (Applied Biosystems, Foster City, Calif.). Resultant sequence data was assembled and edited using Sequencher v4.1 software (GeneCodes Corporation, Ann Arbor, Mich.).

Transformation of electrocompetent E. coli cells W3110 (ATCC, Manassas, Calif.) was done with 1 ml sequencing DNA and 40 ml of W3110 cells. The cells were electropulsed at 2.0 kV, 25 mF and 400 ohms. Following electroporation, 1.0 ml SOC (2% BactoI Tryptone (Difco, Detroit, Mich.), 0.5% yeast extract (Difco), 10 mM NaCl, 2.5 mM KCl, 10 mM MgCl2, 10 mM MgSO4, 20 mM glucose) was added to the cells. After incubation for one hour at 37° C., the cells were plated in one aliquot on LB Kan plates (LB broth (Lennox), 1.8% Bactoa Agar (Difco), 30 mg/L kanamycin).

Individual clones harboring the correct expression construct for human ztnf11 were identified by expression. Cells were grown in Superbroth II (Becton Dickinson) with 30 mg/ml of kanamycin overnight. 50 ml of the overnight culture was used to inoculate 2 ml of fresh Superbroth II+30 mg/ml kanamycin. Cultures were grown at 37° C., shaking for 2 hours. 1 ml of the culture was induced with 1 mM IPTG. 2-4 hours later the 250 ml of each culture was mixed with 250 ml Thorner buffer with 5% bME and dye (8M urea, 100 mM Tris pH7.0, 10% glycerol, 2 mM EDTA, 5% SDS). Samples were heated at 70° C. for 10 minutes. 20 ml were loaded per lane on a 4%-12% PAGE gel (Invitrogen). Gels were run in 1×MES buffer. The positive clones were designated ztnf11/pTAP170. The polynucleotide sequence of MBP-ztnf11 fusion within ztnf11/pTAP170 is shown in SEQ ID NO:33, and the corresponding polypeptide sequence of the MBP-ztnf11 fusion is shown in SEQ ID NO:34.

### Example 16

### Bacterial Expression of Human ztnf11

The positive clone was used to inoculate an overnight starter culture of Superbroth II (Becton Dickinson) with 30 mg/ml of kanamycin. The starter culture was used to inoculate 2 2L-baffled flasks each filled with 500 ml of Superbroth. II+Kan. Cultures shook at 37° C. at 250 rpm until the OD600 reached 2.4. At this point, the cultures were induced with 1 mMIPTG. Cultures grew for four more hours at 37° C., 250 rpm then were harvested via centrifugation. Pellets were saved at −80° C. until transferred to protein purification.

Cell pellets were resuspended in 900 ml of homogenization buffer (20 mM Tris, pH 7.4, 200 mM NaCl) via shaking on a platform shaker at 200 rpm, 37° C. for 1 h. Cells were lysed with three passes through an APV 2000 (APV Homogenizer Group, Wilmington, Mass.) at 8,500-9,000 pounds/in2 keeping the cell suspension chilled to 4° C. An aliquot of the whole cell lysate was retained for future analysis. The homogenized cell suspension was clarified by centrifugation for 1 h at 13,000×g, 4° C. Decanted the supernatant carefully and saved, as well as saved the insoluble pellet. The whole cell lysate was analyzed via SDS-PAGE against the clarified supernatant and the insoluble pellet to assess the partitioning of the target molecule, MBP-ztnf11. The MBP-ztnf11 molecule partitioned to the soluble fraction.

Recombinant target was purified from the clarified lysate by immobilized-metal affinity chromatography (IMAC). Immobilized nickel resin (Qiagen, Valencia, Calif.) was equilibrated with homogenization buffer. Equilibrated resin (10 ml) was combined with the clarified supernatant and batched overnight at 4° C. The lysate/resin slurry was then poured into an empty glass column to pack the resin and to proceed with gravity mediated purification. Flow-through was collected. The column was washed with six column volumes (CV) of homogenization buffer and collected. Proteinwais eluted with homogenization buffer containing 250 mM imidazole (Fluka, Milwaukee, Wis.). Fractions (20×1.5 ml) were collected and analyzed via SDS-PAGE. Pooling of fractions was based on the purity/quality and quantity of MBP-ztnf11 in the analyzed fractions. Pooled fractions were dialyzed against three changes of 4L of PBS (7 mM Na2HPO4, 1.5 mM KH2PO4, 137 mM NaCl, 2.7 mM KCl, pH 7.3). Pooled fractions were then diluted 1:2 with ultra-pure distilled water and loaded onto a 5 ml HiTrap SP column GE Healthcare Pharmacia, Piscataway, N.J.) at a flow rate of 120 cm/h. The column was washed with 25 mM Na-acetate, pH 5.5, 20 mM NaCl. Protein was eluted with a linear gradient over 10 CV to 25 mM Na-acetate, pH 5.5, 1M NaCl. Collected flow-through and 1 ml eluted fractions. Analyzed the flow-through and elution fractions via SDS-PAGE. Target molecule, MBP-ztnf11, was in the flow-through while some impurities were in the elution fractions. Concentrated the flow-through 2-fold prior to dialysis against PBS. The final product was 0.2 mm filtered, analyzed via SDS-PAGE and Western blot prior to aliquoting and storage at −8020 C. according to standard procedures.

### Example 17

### Polyclonal Antibodies

Polyclonal antibodies are prepared by immunizing 2 female New Zealand white rabbits with the purified recombinant protein huztnf11/MBP-6H. The rabbits are each given an initial intraperitoneal (ip) injection of 200 μg of purified protein in Complete Freund's Adjuvant followed by booster ip injections of 100 μg peptide in Incomplete Freund's Adjuvant every three weeks. Seven to ten days after the administration of the second booster injection (3 total injections), the animals are bled and the serum is collected. The animals are then boosted and bled every three weeks.

### Example 18

### Purification of Polyclonal Antibodies to ztnf11 and Use in Western Blots

The immunoglobulin fraction from the sera of rabbits immunized with ztnf11/MBP-6H was purified on a column of Protein A-agarose. Briefly, a column of protein A-agarose was equilibrated with phosphate buffered saline, pH 7.3 (PBS) and ztnf11 immunized rabbit serum was run over the column three times to maximize the potential for binding of the rabbit IgG to the immobilized protein A. The column was rinsed well with PBS and the bound protein eluted with 2 column volumes of 0.1 M glycine buffer, pH 3.4. The solution containing the eluted IgG protein was quickly adjusted to pH 7.0-8.0. The antibodies are then be utilized immediately or the buffer changed to PBS by dialysis or other suitable method known in the art.

### Example 19

### Evaluation of Rabbit Anti-ztnf11/MBP-6H Antibody by Western Blot

Cells which were candidates for containing ztnf11 protein were lysed with a buffer containing, 20 mM Tris, pH 7.4, 1% Triton X-100, 0.5% IGEPAL CA-630, 1 mM EDTA, 1 mM EGTA, and a cocktail of protease inhibitors (“Complete Protease Inhibitors”, Roche Molecular Biochemicals, Indianapolis, Ind.) and disrupted using a microprobe sonicator. The samples were centrifuged at 14,000×g for 20 minutes and the supernatant used to make up samples for SDS-PAGE using 4× LDS-sample buffer (Invitrogen, Carlsbad, Calif.). Aliquots of the cell or tissue extracts were run on 4-12% Bis-Tris polyacrylamide gels using a MES running buffer system (Invitrogen, Carlsbad, Calif.). The gels were transfered to nitrocellulose membranes and used in blotting procedures. The membranes were blocked for 1 hour in a buffer containing 2.5% Blocking Solution (Roche, Indianapolis, Ind.) in phosphate buffered saline, pH 7.4 (PBS) before a 2 hour incubation with Rabbit anti-ztnf11/MBP-6H immunoglobulin (0.1 ug/ml) in 1% Blocking solution/ PBS. The membranes were then incubated for 1 hour with HRP-conjugated Goat anti-Rabbit IgG (Santa Cruz Biotechnology, Inc., Santa Cruz, Calif., 1:2000) in 1% Blocking solution/PBS. The membranes were then rinsed 4 times with blocking solution and then the bound IgG/HRP conjugate was visualized by chemoluminesence and x-ray film detection.

Primary cells and Cell lines were: RPMI 7951, SKLu1 without or with PMA stimulation, U937 stimulated with IL-17(24 h), Raji, MCF-7, THP-1, Malme 3M, HL-60, Granta 519, Y-79, A431, Mouse primary splenocytes, and Mouse primary lymphocytes.

Immunoreactive bands were observed in the lysates from U937 and HL-60 cells at ˜33 kDa on SDS-polyacrylamide gels (the predicted size for ztnf11). These two cell lines also showed the strongest signal when probed for ztnf11 mRNA. A ˜15 kDa band was also observed in the lysate from activated U937 cells. This band may be a proteolytic fragment of ztnf11.

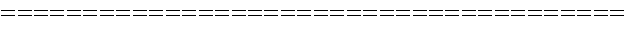

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. An isolated polypeptide comprising the amino acid sequence of residues 116 to 309 of SEQ ID NO:2.

2. The isolated polypeptide according to claim 1, wherein the polypeptide comprises the amino acid sequence selected from:
a) residues 86 to 309 of SEQ ID NO:2;
b) residues 79 to 309 of SEQ ID NO:2;
c) residues 51 to 309 of SEQ ID NO:2;
d) residues 116 to 309 of SEQ ID NO:2;
e) residues 115 to 309 of SEQ ID NO:2; and
f) residues 1 to 309 of SEQ ID NO:2
wherein the polypeptide is at least 80% identical to the amino acid sequence of a), b), c), d), or e).

3. The isolated polypeptide according to claim 2, wherein the polypeptide is at least 85% identical to the amino acid sequence of a), b), c), or d).

4. The isolated polypeptide according to claim 1 wherein the polypeptide forms a multimer selected from
a) a homodimer;
b) a heterodimer;
c) a homotrimer;
d) a heterodimer;
e) a homomultimer; and
f) a heteromultimer.

5. The isolated polypeptide according to claim 1, wherein the polypeptide is covalently linked to an affinity tag or to an immunoglobulin constant region.

6. The isolated polypeptide according to claim 1, wherein the polypeptide induces an inflammatory response.

7. An isolated polynucleotide, wherein the polynucleotide encodes a polypeptide comprising the amino acid sequence selected from:
a) residues 116 to 309 of SEQ ID NO:2;
b) residues 115 to 309 of SEQ ID NO:2;
c) residues 86 to 309 of SEQ ID NO:2;
d) residues 79 to 309 of SEQ ID NO:22;
e) residues 51 to 309 of SEQ ID NO:2; and
f) residues 1 to 309 of SEQ ID NO:2.

8. The isolated polynucleotide according to claim 7, wherein the polypeptide consists of the amino acid sequence.

9. An expression vector comprising the following operably linked elements:
a transcription promoter;
a DNA segment encoding the polypeptide according to claim 1; and
a transcription terminator.

10. The expression vector according to claim 9, wherein the polypeptide comprises an affinity tag or an immunoglogulin constant region.

11. An antibody that specifically binds to the polypeptide according to claim 1.

12. An antibody that specifically binds to the polypeptide according to claim 2.

13. A method of producing an antibody or an antibody fragment, comprising the following steps in order:
inoculating an animal with a polypeptide selected from the group consisting of:
a) a polypeptide consisting of the amino acid sequence from residue 116 to 309 of SEQ ID NO:2;
b) a polypeptide consisting of the amino acid sequence from residue 86 to 309 of SEQ ID NO:2;
c) a polypeptide consisting of the amino acid sequence from reside 79 to 309 of SEQ ID NO:2;
d) a polypeptide consisting of the amino acid sequence from residue 51 to 309 of SEQ ID NO:2; and
e) a polypeptide consisting of the amino acid sequence from residue 1 to 309 of SEQ ID NO:2;
wherein the polypeptide elicits an immune response in the animal to produce the antibody; and isolating the antibody from the animal.

14. An antibody produced by the method of claim 13, which binds to residues 1 to 309 of SEQ ID NO:2.

15. A method of inhibiting or reducing inflammation associated with an autoimmune disease, comprising administering to a mammal with the autoimmune disease a therapeutic amount of an antibody, wherein the antibody specifically binds to the polypeptide selected from:
a) residues 116 to 309 of SEQ ID NO:2;
b) residues 115 to 309 of SEQ ID NO:2;
c) residues 86 to 309 of SEQ ID NO:2;
d) residues 79 to 309 of SEQ ID NO:22;
e) residues 51 to 309 of SEQ ID NO:2; and
f) residues 1 to 309 of SEQ ID NO:2.

16. The method according to claim 15, wherein the autoimmune disease is systemic lupus erythomatosis, myasthenia gravis, multiple sclerosis, or rheumatoid arthritis.

17. A method of inhibiting or reducing inflammation associated with an asthma, bronchitis, or emphysema, comprising administering to a mammal with the asthma, bronchitis, or emphysema a therapeutic amount of an antibody, wherein the antibody specifically binds to the polypeptide selected from:
a) residues 116 to 309 of SEQ ID NO:2;
b) residues 115 to 309 of SEQ ID NO:2;
c) residues 86 to 309 of SEQ ID NO:2;
d) residues 79 to 309 of SEQ ID NO:22;
e) residues 51 to 309 of SEQ ID NO:2; and
f) residues 1 to 309 of SEQ ID NO:2.

18. A method of reducing joint pain, swelling, stiffness, or anemia, comprising comprising administering to a mammal with the joint pain, swelling, stiffness, or anemia a therapeutic amount of an antibody, wherein the antibody specifically binds to the polypeptide selected from:
a) residues 116 to 309 of SEQ ID NO:2;
b) residues 115 to 309 of SEQ ID NO:2;
c) residues 86 to 309 of SEQ ID NO:2;
d) residues 79 to 309 of SEQ ID NO:22;
e) residues 51 to 309 of SEQ ID NO:2; and
f) residues 1 to 309 of SEQ ID NO:2.

19. An antibody or antibody fragment that specifically binds to the polypeptide according to claim l,wherein the antibody or antibody fragment is:
a) a polyclonal antibody,
b) a monoclonal antibody;
c) a murine monoclonal antibody;and
d) a humanized antibody derived from c).

20. An isolated polypeptide comprising the amino acid sequence of residues 116 to 267 of SEQ ID NO:2.

21. The isolated polypeptide according to claim 20, wherein the polypeptide comprises residues 116 to 272 of SEQ ID NO:2, and wherein the polypeptide is at least 80% identical to the amino acid sequence of residues 116 to 272 of SEQ ID NO:2.

22. The isolated polypeptide according to claim 21, wherein the polypeptide is at least 85% identical to the amino acid sequence residues 116 to 272 of SEQ ID NO:2.

23. The isolated polypeptide according to claim 20 wherein the polypeptide forms a multimer selected from:
a) a homodimer;
b) a heterodimer;
c) a homotrimer;
d) a heterodimer;
e) a homomultimer; and
f) a heteromultimer.

24. The isolated polypeptide according to claim 20, wherein the polypeptide is covalently linked to an affinity tag or to an immunoglobulin constant region.

25. The isolated polypeptide according to claim 26, wherein the polypeptide induces an inflammatory response.

26. An isolated polynucleotide, wherein the polynucleotide encodes the polypeptide according to claim 20.

27. The isolated polynucleotide according to claim 26, wherein the polypeptide consists of the amino acid sequence.

28. The isolated polynucleotide according to claim 26, wherein the polynucleotide encodes a polypeptide comprising residues 116 to 272 of SEQ ID NO:2.

29. The isolated polynucleotide according to claim 28, wherein the polypeptide consists of the amino acid sequence.

30. An expression vector comprising the following operably linked elements:
a transcription promoter;
a DNA segment encoding the polypeptide according to claim 20; and
a transcription terminator.

31. The expression vector according to claim 30, wherein the polypeptide comprises an affinity tag or an immunoglogulin constant region.

32. An antibody that specifically binds to the polypeptide according to claim 20.

33. An antibody that specifically binds to the polypeptide according to claim 21.

34. A method of producing an antibody or an antibody fragment, comprising the following steps in order:
inoculating an animal with a polypeptide selected from the group consisting of:
a) a polypeptide consisting of the amino acid sequence from residue 116 to 267 of SEQ ID NO:2; and
b) a polypeptide consisting of the amino acid sequence from residue 116 to 272 of SEQ ID NO:2;
wherein the polypeptide elicits an immune response in the animal to produce the antibody; and isolating the antibody from the animal.

35. An antibody produced by the method of claim 34, which binds to residues 116 to 267 of SEQ ID NO:2.

36. A method of inhibiting or reducing inflammation associated with an autoimmune disease, comprising administering to a mammal with the autoimmune disease a therapeutic amount of an antibody, wherein the antibody specifically binds to the polypeptide selected from:
a) residues 116 to 262 of SEQ ID NO:2; and
b) residues 116 to 272 of SEQ ID NO:2.

37. The method according to claim 46, wherein the autoimmune disease is systemic lupus erythomatosis, myasthenia gravis, multiple sclerosis, or rheumatoid arthritis.

38. A method of inhibiting or reducing inflammation associated with an asthma, bronchitis, or emphysema, comprising administering to a mammal with the asthma, bronchitis, or emphysema a therapeutic amount of an antibody, wherein the antibody specifically binds to the polypeptide selected from:
a) residues 116 to 267 of SEQ ID NO:2; and
b) residues 116 to 272 of SEQ ID NO:2.

39. A method of reducing joint pain, swelling, stiffness, or anemia, comprising administering to a mammal with the joint pain, swelling, stiffness, or anemia a therapeutic amount of an antibody, wherein the antibody specifically binds to the polypeptide selected from:
a) residues 116 to 267 of SEQ ID NO:2; and
b) residues 116 to 272 of SEQ ID NO:2;.

40. An antibody or antibody fragment that specifically binds to the polypeptide according to claim 20,wherein the antibody or antibody fragment is:
a) a polyclonal antibody,
b) a monoclonal antibody;
c) a murine monoclonal antibody; and
d) a humanized antibody derived from c).

41. An isolated polypeptide comprising the amino acid sequence of residues 51 to 120 of SEQ ID NO:12.

42. The isolated polypeptide according to claim 41, wherein the polypeptide comprises residues 1 to 120 of SEQ ID NO:2, and wherein the polypeptide is at least 80% identical to the amino acid sequence of residues 1 to 120.

43. The isolated polypeptide according to claim 42, wherein the polypeptide is at least 85% identical to the amino acid sequence of residues 1 to 120.

44. The isolated polypeptide according to claim 41 wherein the polypeptide forms a multimer selected from:
a) a homodimer;
b) a heterodimer;
c) a homotrimer;
d) a heterodimer;
e) a homomultimer; and
f) a heteromultimer.

45. The isolated polypeptide according to claim 41, wherein the polypeptide is covalently linked to an affinity tag or to an immunoglobulin constant region.

46. The isolated polypeptide according to claim 41, wherein the polypeptide limits the inhibition of an inflammatory response.

47. An isolated polynucleotide, wherein the polynucleotide encodes the polypeptide according to claim 41.

48. The isolated polynucleotide according to claim 47, wherein the polypeptide consists of resides 51 to 120 of SEQ ID NO:12.

49. The isolated polynucleotide according to claim 47, wherein the polynucleotide comprises residues 1 to 120 of SEQ ID NO:12.

50. An expression vector comprising the following operably linked elements:
a transcription promoter;
a DNA segment encoding the polypeptide according to claim 41; and
a transcription terminator.

51. The expression vector according to claim 50, wherein the polypeptide comprises an affinity tag or an immunoglogulin constant region.

52. An antibody that specifically binds to the polypeptide according to claim 41.

53. An antibody that specifically binds to the polypeptide according to claim 42.

54. A method of producing an antibody or an antibody fragment, comprising the following steps in order:
inoculating an animal with a polypeptide selected from the group consisting of:
a) a polypeptide consisting of the amino acid sequence from residue 51 to 120 of SEQ ID NO:12; and
b) a polypeptide consisting of the amino acid sequence from residue 1 to 120 of SEQ ID NO:2;
wherein the polypeptide elicits an immune response in the animal to produce the antibody; and isolating the antibody from the animal.

55. An antibody produced by the method of claim 54, which binds to residues 1 to 120 of SEQ ID NO:12.

56. A method limiting the reduction of inflammation associated with an autoimmune disease, comprising administering to a mammal with the autoimmune disease a therapeutic amount of an antibody, wherein the antibody specifically binds to the polypeptide selected from:
a) residues 51 to 120 of SEQ ID NO:12;
b) residues 1 to 120 of SEQ ID NO:12;.

57. The method according to claim 56, wherein the autoimmune disease is systemic lupus erythomatosis, myasthenia gravis, multiple sclerosis, or rheumatoid arthritis.

58. A method of limiting the reduction of inflammation associated with an asthma, bronchitis, or emphysema, comprising administering to a mammal with the asthma, bronchitis, or emphysema a therapeutic amount of an antibody, wherein the antibody specifically binds to the polypeptide selected from:
a) residues 51 to 309 of SEQ ID NO:12; and
b) residues 1 to 120 of SEQ ID NO:12.

59. A method of limiting the reduction of inflammation in joint pain, swelling, stiffness, or anemia, comprising comprising administering to a mammal with the joint pain, swelling, stiffness, or anemia a therapeutic amount of an antibody, wherein the antibody specifically binds to the polypeptide selected from:
a) residues 51 to 120 of SEQ ID NO:12; and
b) residues 1 to 120 of SEQ ID NO:12;.

60. An antibody or antibody fragment that specifically binds to the polypeptide according to claim 41,wherein the antibody or antibody fragment is:
a) a polyclonal antibody,
b) a monoclonal antibody;
c) a murine monoclonal antibody; and
d) a humanized antibody derived from c).


SEQUENCE LISTING (34 sequences)

SEQ ID NO: 1 - DNA, 1009 bp, Homo sapiens
  CDS (26)...(952)
gggccccggtggtcctagggaccccatggccatggttgagcggccgaggcccgagtgggcctcgtatcacaactgcaacagcaacagctgccaggacctgggcaactctgtcctgttgctgctgggcctcatcatctgcattaacattagcatcaatatagtgaccctgctctggagccgattccgtggtgtcttataccaagtgttccatgataccatttgtgagaaagaagctcccaagtcatcattactcagaaagcagacccagccccctaagaagcagagttctcctgcagtccatcttcggtgcaccatggaccctgtgatgatgactgtgtccccgcccccagctcaccgccatcgccgtcgaggctctcccacacgctgtgctcactgcccagtagcttgggctcctgacactgatgacgagaagcctcatcagtacccagccatctgctcctaccactgggatgtccctgaggactgggaaggcttccaacacactcaggggacctgggttccctggtctcaggatgccccagagtcccctccccagaccatccgcttccagcctaccgtagaggaaaggcccctcaaaacaggcatatggtccgagctgggcctaagggcctatgtgtatcctgtgaaccccccacctcccagccctgaggctcctagccacaagaacggtggggagggggcggtgccagaggcagaggcggctcagtaccagcctgtcccagctcccaccctgggcccagcagtcatccctgaattttcccggcaccgctcctcaggccgaatagtgtatgatgcccgggacatgagacggcggcttcgggaactgacccgggaggtggaggccctgtccggctgctaccccctagcctctggatccagcactgccgaggagacaagcaagaattgggtgtaccgttccctaactgggaggtgactggaaaaaaataaaaaggagagaggaaaaaaaaaaaaaaaaaaaaaaaaaaaa

SEQ ID NO: 2 - protein, 309 aa, Homo sapiens
MAMVERPRPEWASYHNCNSNSCQDLGNSVLLLLGLIICINISINIVTLLWSRFRGVLYQVFHDTICEKEAPKSSLLRKQTQPPKKQSSPAVHLRCTMDPVMMTVSPPPAHRHRRRGSPTRCAHCPVAWAPDTDDEKPHQYPAICSYHWDVPEDWEGFQHTQGTWVPWSQDAPESPPQTIRFQPTVEERPLKTGIWSELGLRAYVYPVNPPPPSPEAPSHKNGGEGAVPEAEAAQYQPVPAPTLGPAVIPEFSRHRSSGRIVYDARDMRRRLRELTREVEALSGCYPLASGSSTAEETSKNWVYRSLTGR

SEQ ID NO: 3 - DNA, 927 bp, Artificial Sequence
  Computationally derived degenerate polynucleotide sequence
atggcnatggtngarmgnccnmgnccngartgggcnwsntaycayaaytgyaaywsnaaywsntgycargayytnggnaaywsngtnytnytnytnytnggnytnathathtgyathaayathwsnathaayathgtnacnytnytntggwsnmgnttymgnggngtnytntaycargtnttycaygayacnathtgygaraargargcnccnaarwsnwsnytnytnmgnaarcaracncarccnccnaaraarcarwsnwsnccngcngtncayytnmgntgyacnatggayccngtnatgatgacngtnwsnccnccnccngcncaymgncaymgnmgnmgnggnwsnccnacnmgntgygcncaytgyccngtngcntgggcnccngayacngaygaygaraarccncaycartayccngcnathtgywsntaycaytgggaygtnccngargaytgggarggnttycarcayacncarggnacntgggtnccntggwsncargaygcnccngarwsnccnccncaracnathmgnttycarccnacngtngargarmgnccnytnaaracnggnathtggwsngarytnggnytnmgngcntaygtntayccngtnaayccnccnccnccnwsnccngargcnccnwsncayaaraayggnggngarggngcngtnccngargcngargcngcncartaycarccngtnccngcnccnacnytnggnccngcngtnathccngarttywsnmgncaymgnwsnwsnggnmgnathgtntaygaygcnmgngayatgmgnmgnmgnytnmgngarytnacnmgngargtngargcnytnwsnggntgytayccnytngcnwsnggnwsnwsnacngcngargaracnwsnaaraaytgggtntaymgnwsnytnacnggnmgn

SEQ ID NO: 4 - protein, 11 aa, Artificial Sequence
  TNF polypeptide consensus motif
XXXXXXGXXXX

SEQ ID NO: 5 - protein, 11 aa, Homo sapiens
GIWSELGLRAY

SEQ ID NO: 6 - protein, 224 aa, Homo sapiens
QSSPAVHLRCTMDPVMMTVSPPPAHRHRRRGSPTRCAHCPVAWAPDTDDEKPHQYPAICSYHWDVPEDWEGFQHTQGTWVPWSQDAPESPPQTIRFQPTVEERPLKTGIWSELGLRAYVYPVNPPPPSPEAPSHKNGGEGAVPEAEAAQYQPVPAPTLGPAVIPEFSRHRSSGRIVYDARDMRRRLRELTREVEALSGCYPLASGSSTAEETSKNWVYRSLTGR

SEQ ID NO: 7 - protein, 194 aa, Homo sapiens
GSPTRCAHCPVAWAPDTDDEKPHQYPAICSYHWDVPEDWEGFQHTQGTWVPWSQDAPESPPQTIRFQPTVEERPLKTGIWSELGLRAYVYPVNPPPPSPEAPSHKNGGEGAVPEAEAAQYQPVPAPTLGPAVIPEFSRHRSSGRIVYDARDMRRRLRELTREVEALSGCYPLASGSSTAEETSKNWVYRSLTGR

SEQ ID NO: 8 - protein, 231 aa, Homo sapiens
QTQPPKKQSSPAVHLRCTMDPVMMTVSPPPAHRHRRRGSPTRCAHCPVAWAPDTDDEKPHQYPAICSYHWDVPEDWEGFQHTQGTWVPWSQDAPESPPQTIRFQPTVEERPLKTGIWSELGLRAYVYPVNPPPPSPEAPSHKNGGEGAVPEAEAAQYQPVPAPTLGPAVIPEFSRHRSSGRIVYDARDMRRRLRELTREVEALSGCYPLASGSSTAEETSKNWVYRSLTGR

SEQ ID NO: 9 - DNA, 1060 bp, Mus musculus
aggctagtgggcattaggggtagtgggctgggggcctaagggccctggtggccctagggaccaccatggccatggctgagcggcctcggcctgggtgggcctcgtatcaaaaccccaacatcaacaactgccaggacatgggaaactccattctgctgctcttgggtcttatcgtctgtattaacattggcatcaatttggtgaccctgctctggagccgaatccgcatcctcttacaccgaatgttccatatcatttgtgaaaaagaaaccagtacttcacttgggaagctggtccagcccttgaggaagcagacctaccccaaagtccatcttcgatgtaccatggaccctgtgaagatgactgtgacccccccacccactcgtcgtcgttaccgccatcgagcgcctccatctcgccgtgcccgctgccccatagcttgggcccctgatactgatgatgagaaacctccacatcagcaccctgcaatctgttcccgccactggaatgactctaaggattgggaaggctttcaatctatgcaggaggtgtgggaaccttggactcaggatggtctagagcaacctccccagaccatccgcttccagccagcagtagaggccaggcctctcaaatcggaaatccggtcagaccagggcctagaggcctatgtgtatactgtaaatcctccacctcccagtccagaggctttgagccataaaaacaatgcggcagggtcagggggatgtgttgagggtgagcaagcacagggccagcctgtctcaccatcctttctgggtccagcaaatgtccctgaaataccccggcgccattcatcaggccgaatagtctatgatgcccgagatgtgaggagaaggttacgagaactcacccaggaggtggaggctctgtcccactgttaccccttggtctctggatccagcgcagctgaggggacaggcaaagattgggtgtatcgtcccctgaagggaaggtgactggggacaaaaaaataaaaaataaaaaagagaaaaaaaaaaaaaaaaaaaaaaaaaaaaaa

SEQ ID NO: 10 - protein, 310 aa, Mus musculus
MAMAERPRPGWASYQNPNINNCQDMGNSILLLLGLIVCINIGINLVTLLWSRIRILLHRMFHIICEKETSTSLGKLVQPLRKQTYPKVHLRCTMDPVKMTVTPPPTRRRYRHRAPPSRRARCPIAWAPDTDDEKPPHQHPAICSRHWNDSKDWEGFQSMQEVWEPWTQDGLEQPPQTIRFQPAVEARPLKSEIRSDQGLEAYVYTVNPPPPSPEALSHKNNAAGSGGCVEGEQAQGQPVSPSFLGPANVPEIPRRHSSGRIVYDARDVRRRLRELTQEVEALSHCYPLVSGSSAAEGTGKDWVYRPLKGR

SEQ ID NO: 11 - DNA, 363 bp, Homo sapiens
  CDS (1)...(363)
atggccatggttgagcggccgaggcccgagtgggcctcgtatcacaactgcaacagcaacagctgccaggacctgggcaactctgtcctgttgctgctgggcctcatcatctgcattaacattagcatcaatatagtgaccctgctctggagccgattccgtggtgtcttataccaagtgttccatgataccatttgtgagaaaggccgaatagtgtatgatgcccgggacatgagacggcggcttcgggaactgacccgggaggtggaggccctgtccggctgctaccccctagcctctggatccagcactgccgaggagacaagcaagaattgggtgtaccgttccctaactgggaggtga

SEQ ID NO: 12 - protein, 120 aa, Homo sapiens
MAMVERPRPEWASYHNCNSNSCQDLGNSVLLLLGLIICINISINIVTLLWSRFRGVLYQVFHDTICEKGRIVYDARDMRRRLRELTREVEALSGCYPLASGSSTAEETSKNWVYRSLTGR

SEQ ID NO: 13 - protein, 152 aa, Homo sapiens
GSPTRCAHCPVAWAPDTDDEKPHQYPAICSYHWDVPEDWEGFQHTQGTWVPWSQDAPESPPQTIRFQPTVEERPLKTGIWSELGLRAYVYPVNPPPPSPEAPSHKNGGEGAVPEAEAAQYQPVPAPTLGPAVIPEFSRHRSSGRIVYDARDM

SEQ ID NO: 14 - protein, 157 aa, Homo sapiens
GSPTRCAHCPVAWAPDTDDEKPHQYPAICSYHWDVPEDWEGFQHTQGTWVPWSQDAPESPPQTIRFQPTVEERPLKTGIWSELGLRAYVYPVNPPPPSPEAPSHKNGGEGAVPEAEAAQYQPVPAPTLGPAVIPEFSRHRSSGRIVYDARDMRRRLR

SEQ ID NO: 15 - protein, 120 aa, Homo sapiens; identical to SEQ ID NO: 12

SEQ ID NO: 16 - protein, 70 aa, Homo sapiens
SRFRGVLYQVFHDTICEKGRIVYDARDMRRRLRELTREVEALSGCYPLASGSSTAEETSKNWVYRSLTGR

SEQ ID NO: 17 - protein, 259 aa, Homo sapiens
SRFRGVLYQVFHDTICEKEAPKSSLLRKQTQPPKKQSSPAVHLRCTMDPVMMTVSPPPAHRHRRRGSPTRCAHCPVAWAPDTDDEKPHQYPAICSYHWDVPEDWEGFQHTQGTWVPWSQDAPESPPQTIRFQPTVEERPLKTGIWSELGLRAYVYPVNPPPPSPEAPSHKNGGEGAVPEAEAAQYQPVPAPTLGPAVIPEFSRHRSSGRIVYDARDMRRRLRELTREVEALSGCYPLASGSSTAEETSKNWVYRSLTGR

SEQ ID NO: 18 - DNA, 23 bp, Artificial Sequence
  oligonucleotide, ZC21,195
gaggagaccataacccccgacag

SEQ ID NO: 19 - DNA, 23 bp, Artificial Sequence
  Oligonucleotide ZC 21,196
catagctcccaccacacgatttt

SEQ ID NO: 20 - DNA, 20 bp, Artificial Sequence
  Oligonucleotide ZC 47,124
ctggagccgattccgtggtg

SEQ ID NO: 21 - DNA, 18 bp, Artificial Sequence
  Oligonucleotide ZC 47,127
ccgaagccgccgtctcat

SEQ ID NO: 22 - DNA, 21 bp, Artificial Sequence
  Oligonucleotide ZC 47,363
accccaacatcaacaactgcc

SEQ ID NO: 23 - DNA, 19 bp, Artificial Sequence
  Oligonucleotide ZC 47,364
tggcggtaacgacgacgag

SEQ ID NO: 24 - DNA, 93 bp, Artificial Sequence
  Oligonucleotide ZC 47,213
ctcagccaggaaatccatgccgagttgagacgcttccgtagatctgattataaggatgatgatgataagggatccgtagcttgggctcctgac

SEQ ID NO: 25 - DNA, 57 bp, Artificial Sequence
  Oligonucleotide ZC 47,214
tacaaccccagagctgttttaaggcgcgcctctagattatcacctcccagttaggga

SEQ ID NO: 26 - DNA, 802 bp, Homo sapiens
agccgattccgtggtgtcttataccaagtgttccatgataccatttgtgagaaagaagctcccaagtcatcattactcagaaagcagacccagccccctaagaagcagagttctcctgcagtccatcttcggtgcaccatggaccctgtgatgatgactgtgtccccgcccccagctcaccgccatcgccgtcgaggctctcccacacgctgtgctcactgcccagtagcttgggctcctgacactgatgacgagaagcctcatcagtacccagccatctgctcctaccactgggatgtccctgaggactgggaaggcttccaacacactcaggggacctgggttccctggtctcaggatgccccagagtcccctccccagaccatccgcttccagcctaccgtagaggaaaggcccctcaaaacaggcatatggtccgagctgggcctaagggcctatgtgtatcctgtgaaccccccacctcccagccctgaggctcctagccacaagaacggtggggagggggcggtgccagaggcagaggcggctcagtaccagcctgtcccagctcccaccctgggcccagcagtcatccctgaattttcccggcaccgctcctcaggccgaatagtgtatgatgcccgggacatgagacggcggcttcgggaactgacccgggaggtggaggccctgtccggctgctaccccctagcctctggatccagcactgccgaggagacaagcaagaattgggtgtaccgttccctaactgggaggtgttttggcggatgagataagattt

SEQ ID NO: 27 - DNA, 58 bp, Artificial Sequence
  Oligonucleotide ZC 47,357
cgcgaattcggtaccgctggttccgcgtggatccagccgattccgtggtgtcttatac

SEQ ID NO: 28 - DNA, 49 bp, Artificial Sequence
  Oligonucleotide ZC 47,358
aaatcttatctcatccgccaaaacacctcccagttagggaacggtacac

SEQ ID NO: 29 - DNA, 40 bp, Artificial Sequence
  Oligonucleotide ZC 19,372
tgtcgatgaagccctgaaagacgcgcagactaattcgagc

SEQ ID NO: 30 - DNA, 60 bp, Artificial Sequence
  Oligonucleotide ZC 19,351
acgcgcagactaattcgagctcccaccatcaccatcaccacgcgaattcggtaccgctgg

SEQ ID NO: 31 - DNA, 60 bp, Artificial Sequence
  Oligonucleotide ZC 19,352
actcactatagggcgaattgcccgggggatccacgcggaaccagcggtaccgaattcgcg

SEQ ID NO: 32 - DNA, 42 bp, Artificial Sequence
  Oligonucleotide ZC 19,371
acggccagtgaattgtaatacgactcactatagggcgaattg

SEQ ID NO: 33 - DNA, 1956 bp, Homo sapiens
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcccaccatcaccatcaccacgcgaattcggtaccgctggttccgcgtggatccagccgattccgtggtgtcttataccaagtgttccatgataccatttgtgagaaagaagctcccaagtcatcattactcagaaagcagacccagccccctaagaagcagagttctcctgcagtccatcttcggtgcaccatggaccctgtgatgatgactgtgtccccgcccccagctcaccgccatcgccgtcgaggctctcccacacgctgtgctcactgcccagtagcttgggctcctgacactgatgacgagaagcctcatcagtacccagccatctgctcctaccactgggatgtccctgaggactgggaaggcttccaacacactcaggggacctgggttccctggtctcaggatgccccagagtcccctccccagaccatccgcttccagcctaccgtagaggaaaggcccctcaaaacaggcatatggtccgagctgggcctaagggcctatgtgtatcctgtgaaccccccacctcccagccctgaggctcctagccacaagaacggtggggagggggcggtgccagaggcagaggcggctcagtaccagcctgtcccagctcccaccctgggcccagcagtcatccctgaattttcccggcaccgctcctcaggccgaatagtgtatgatgcccgggacatgagacggcggcttcgggaactgacccgggaggtggaggccctgtccggctgctaccccctagcctctggatccagcactgccgaggagacaagcaagaattgggtgtaccgttccctaactgggaggtgttttggcggatga

SEQ ID NO: 34 - protein, 651 aa, Homo sapiens [sequence omitted]